(19) United States
(12) Patent Application Publication
(10) Pub. No.: US 2026/0151473 A1
(43) Pub. Date: Jun. 4, 2026

(54) RSV RNA VACCINES

(71) Applicant: ModernaTX, Inc, Cambridge, MA (US)

(72) Inventors: Giuseppe Ciaramella, Sudbury, MA (US); Kapil Bahl, Medford, MA (US); Amy Espeseth, Chalfont, PA (US); Andrew J. Bett, Lansdale, PA (US); Pedro Cejas, Oreland, PA (US); Lan Zhang, Chalfont, PA (US); Christine Shaw, Reading, MA (US)

(73) Assignee: ModernaTX, Inc, Cambridge, MA (US)

(21) Appl. No.: 19/345,347

(22) Filed: Sep. 30, 2025

Related U.S. Application Data
(63) Continuation of application No. 18/418,127, filed on Jan. 19, 2024, now Pat. No. 12,453,766.
Application No. 18/418,127 is a continuation of application No. 16/965,589, now Pat. No. 11,911,453.
(60) Provisional application No. 62/623,240, filed on Jan. 29, 2018.

(51) Int. Cl. A61K 39/12; A61K 9/00; A61K 39/00

(52) U.S. Cl. CPC A61K 39/12; A61K 9/0019; A61K 2039/53; A61K 2039/55555

(57) ABSTRACT

The disclosure relates to respiratory syncytial virus (RSV) ribonucleic acid (RNA) vaccines as well as methods of using the vaccines and compositions comprising the vaccines. The vaccine can be formulated in a lipid nanoparticle.

### RELATED APPLICATIONS

This application is a continuation of U.S. application Ser. No. 18/418,127, filed Jan. 19, 2024, which is a continuation of U.S. application Ser. No. 16/965,589, filed Jul. 28, 2020, which is a national stage filing under 35 U.S.C. § 371 of international application number PCT/US2019/015412, filed Jan. 28, 2019, which was published under PCT Article 21(2) in English and claims the benefit under 35 U.S.C. § 119(e) of U.S. provisional application No. 62/623,240, filed Jan. 29, 2018, each of which is incorporated by reference herein in its entirety.

### REFERENCE TO AN ELECTRONIC SEQUENCE LISTING

The contents of the electronic sequence listing (M137870101US03-SEQ-JXV.xml; Size: 240,818 bytes; and Date of Creation: Sep. 24, 2025) are herein incorporated by reference in its entirety.

### BACKGROUND

The human respiratory syncytial virus (RSV) is a negative-sense, single-stranded RNA virus of the genus Pneumovirinae and of the family Paramyxoviridae. Symptoms in adults typically resemble a sinus infection or the common cold, although the infection may be asymptomatic. In older adults (e.g., >60 years), RSV infection may progress to bronchiolitis or pneumonia. Symptoms in children are often more severe. It is estimated that in the United States, most children are infected with RSV by the age of three. The RSV virion includes of an internal nucleocapsid comprised of the viral RNA bound to nucleoprotein (N), phosphoprotein (P), and large polymerase protein (L). The nucleocapsid is surrounded by matrix protein (M) and is encapsulated by a lipid bilayer into which the viral fusion (F) and attachment (G) proteins as well as the small hydrophobic protein (SH) are incorporated. The viral genome also encodes two nonstructural proteins (NS1 and NS2), which inhibit type I interferon activity as well as the M-2 protein. Currently, there is no vaccine to prevent primary infection or disease.

### SUMMARY

Provided herein, in some embodiments, are highly immunogenic respiratory syncytial virus (RSV) ribonucleic acid (RNA) (e.g., mRNA) compositions (e.g, vaccines) that elicit potent neutralizing antibodies responses at a dose that is at least 5-fold lower than a control, wherein the control is, for example, a RNA vaccine encoding a membrane-bound DS-CAV1-stabilized prefusion F protein of RSV (e.g., SEQ ID NO: 90 or SEQ ID NO: 92). The envelope of RSV contains three surface glycoproteins: F, G, and SH. The G and F proteins are protective antigens and targets of neutralizing antibodies. The F protein, however, is more conserved across RSV strains and types (A and B). RSV F is a type I viral fusion protein that structurally rearranges from a metastable prefusion form to a highly stable postfusion form. Although targets for neutralizing monoclonal antibodies exist on the postfusion conformation of F protein, the neutralizing Ab response primarily targets the F protein prefusion conformation in people naturally infected with RSV (Magro M et al., Proc Natl Acad Sci USA; 109(8):3089-94, 2012; Ngwuta J O et al., Sci Transl Med 2015; 7(309):309ra162). Thus, the present disclosure focuses, in some aspects, on RNA vaccines encoding RSV F protein (e.g., F protein trimer) stabilized in its prefusion conformation. These RNA vaccines encode antigens that elicit an even greater immune response and that are even more stable in the prefusion form, relative to previously described recombinant RSV F trimers, which includes RSV F trimers containing the “DS-CAV1” substitutions (155C, 290C, 190F, and 207L).

In some aspects, the RSV vaccines of the present disclosure include a RNA comprising an open reading frame (ORF) encoding a RSV antigen, wherein intramuscular (IM) administration of a therapeutically effective amount of the vaccine to a subject induces in the subject a neutralizing antibody titer.

Some aspects of the present disclosure provide respiratory syncytial virus (RSV) vaccines comprising a ribonucleic acid (RNA) that comprises an open reading frame (ORF) encoding an RSV antigen, wherein the ORF comprises (or consists of, or consists essentially of) a sequence that is at least 95% (e.g., at least 96%, at least 97%, at least 98%, at least 99%) or 95%-99% identical to a sequence identified by any one of SEQ ID NOS: 3, 7, 10, 13, 16, 19, 22, 24, 26, 28, 30, 32, 34, 37, 40, 43, 46, 49, 52, 54, 56, 58, 60, 63, 66, 69, 75, 96, or 97. In some aspects, the vaccine is formulated in a lipid nanoparticle. In some embodiments, the ORF comprises a sequence that is at least 98% identical to a sequence identified by any one of SEQ ID NOS: 3, 7, 10, 13, 16, 19, 22, 24, 26, 28, 30, 32, 34, 37, 40, 43, 46, 49, 52, 54, 56, 58, 60, 63, 66, 69, 75, 96, or 97. In some embodiments, the RNA comprises an ORF encoding an RSV antigen, wherein the ORF comprises a sequence identified by (is 100% identical to) any one of SEQ ID NOS: 3, 7, 10, 13, 16, 19, 22, 24, 26, 28, 30, 32, 34, 37, 40, 43, 46, 49, 52, 54, 56, 58, 60, 63, 66, 69, 72, 75, 96 or 97.

In some embodiments, the ORF comprises (or consists of, or consists essentially of) a sequence that is identified by (is 100% identical to) SEQ ID NO: 22. In some embodiments, the ORF comprises a sequence that is identified by (is 100% identical to) SEQ ID NO: 75. In some embodiments, the ORF comprises (or consists of, or consists essentially of) a sequence that is identified by (is 100% identical to) SEQ ID NO: 3. In some embodiments, the ORF comprises (or consists of, or consists essentially of) a sequence that is identified by (is 100% identical to) SEQ ID NO: 52. In some embodiments, the ORF comprises a sequence that is identified by (is 100% identical to) SEQ ID NO: 54. In some embodiments, the ORF comprises a sequence that is identified by (is 100% identical to) SEQ ID NO: 56. In some embodiments, the ORF comprises a sequence that is identified by (is 100% identical to) SEQ ID NO: 58.

Other aspects of the present disclosure provide a RSV vaccine comprising (or consisting of, or consisting essentially of) a RNA that comprises an ORF encoding an RSV antigen, wherein the ORF encodes a sequence identified by any one of SEQ ID NOS: 5, 8, 11, 14, 17, 20, 22, 35, 38, 41, 44, 47, 50, 61, 65, 67, 70, 73, 76. In some aspects, said RSV vaccine is formulated in a lipid nanoparticle.

In some embodiments, the RNA comprises (or consists of, or consists essentially of) an ORF encoding a single chain recombinant RSV F peptide comprising a deletion of wild type RSV F amino acid positions 98-146 and a linker of eight to fourteen amino acids between wildtype RSV F amino acid positions 97 and 147, wherein the recombinant F peptide comprises the following amino acid modifications to stabilize the recombinant RSV F peptide when oligermized to form a trimer in a perfusion conformations: (i) 190F and 207L amino acid substitutions, (ii) 155C and 290C amino acid substitutions, and (iii) one (or more) of (a) 486C and 490C amino acid substitutions; (b) 180C and 186C amino acid substitutions; (c) 486C and 489C amino acid substitutions; (d) 512C and 513C amino acid substitutions; and (e) an 505C amino acid substitution. The amino acid substitutions that introduce additional cysteine amino acid residues may result in either intra-peptide or inter-peptide disulfide bonds, specifically between the recited substitution pairs (e.g., a non-native intra peptide disulfide bond between 155C and 290C or between 180C and 186C; a non-native inter-peptide disulfide bond between 486C and 490C, between 486C and 489C or between 512C and 513C).

In some embodiments, intramuscular (IM) administration of a therapeutically effective amount of the vaccine to a subject induces in the subject a neutralizing antibody titer to (or against) the RSV F.

In some embodiments, the neutralizing antibody titer is at least 5-fold to at least 100-fold (e.g., at least 5-fold, at least 10-fold, at least 15-fold, at least 20-fold, at least 25-fold, or at least 50-fold) higher relative to control. In some embodiments the control is a RNA vaccine encoding a membrane-bound DS-CAV1-stabilized prefusion F protein of RSV. In some embodiments, the control RNA vaccine encoding a membrane-bound DS-CAV1-stabilized prefusion F protein of RSV comprises (or consists of, or consists essentially of) a sequence identified by SEQ ID NO: 90 or SEQ ID NO: 92. In some embodiments, the control is a live attenuated RSV vaccine, an inactivated RSV vaccine, or a protein subunit RSV vaccine.

In some embodiments, the at least 5-fold to at least 100-fold (e.g., at least 5-fold, at least 10-fold, at least 15-fold, at least 20-fold, at least 25-fold, or at least 50-fold) higher neutralizing antibody titer is induced in the subject following administration of a dose of the vaccine that is at least 5-fold (e.g., at least 6-fold, at least 7-fold, at least 8-fold, at least 9-fold, at least 10-fold) lower relative to the control.

In some embodiments, IM administration of a therapeutically effective amount of the vaccine to a subject induces in the subject at least 10-fold, at least 15-fold, at least 20-fold, or at least 25-fold higher prefusion RSV F-specific neutralizing antibody titers relative to the control.

In some embodiments, IM administration of a therapeutically effective amount of the vaccine to a subject confers prophylactic protection at a 5-fold (e.g., at least 6-fold, at least 7-fold, at least 8-fold, at least 9-fold, at least 10-fold) lower dose relative to the control.

In some embodiments, the RSV F neutralizing antibody titer is induced in the subject following fewer than three (e.g., one or two) doses of the vaccine.

In some embodiments, a single dose is of 5 μg-25 μg (e.g., 5 μg, 10 μg, 15 μg, 20 μg, or 25 μg).

In some embodiments, a 5 μg booster dose of the vaccine induces in African green monkeys a NT₅₀ neutralizing antibody titer of about 10³. In some embodiments, a 5 μg booster dose of the vaccine induces in African green monkeys a NT₅₀ neutralizing antibody titer of at least 10³. In some embodiments, a 5 μg booster dose of the vaccine induces in African green monkeys a NT₅₀ neutralizing antibody titer of about 10⁴. In some embodiments, a 5 μg booster dose of the vaccine induces in African green monkeys a NT₅₀ neutralizing antibody titer of at least 10⁴. In some embodiments, the NT₅₀ neutralizing antibody titer (e.g., of about 10³ or about 10⁴) is induced 2 weeks post vaccine administration. Methods of assessing neutralizing antibody titers are known and described, for example, by Zhang L, et al. Design and characterization of a fusion glycoprotein vaccine for Respiratory Syncytial Virus with improved stability. Vaccine 2018; 36: 8119-8130; and Shambaugh C, et al. Development of a high-throughput respiratory syncytial virus fluorescent focus-based microneutralization assay. Clin Vaccine Immunol 2017; 24: e00225-17, each of which is incorporated by reference herein in its entirety.

In some embodiments, the RSV vaccine comprises (a) a ribonucleic acid (RNA) comprising an open reading frame (ORF) encoding two RSV antigens, or (b) two RNAs, each comprising an ORF encoding an RSV antigen.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, the RSV antigen is fused to a scaffold moiety. In some embodiments, the scaffold moiety is selected from the group consisting of: ferritin, encapsulin, lumazine synthase, hepatitis B surface antigen, and hepatitis B core antigen.

In some embodiments, the RNA comprises messenger RNA (mRNA).

In some embodiments, the RNA further comprises a 5′ UTR. In some embodiments, the 5′ UTR comprises a sequence identified by SEQ ID NO: 2 or SEQ ID NO: 77. In some embodiments, the RNA further comprises a 3′ UTR. In some embodiments, the 3′ UTR comprises a sequence identified by SEQ ID NO: 4 or SEQ ID NO: 78.

In some embodiments, the RNA is unmodified. In some embodiments, the RNA comprise at least one modified nucleotide. In some embodiments, at least 80% of the uracil in the ORF comprise a 1-methyl-pseudouridine modification.

Some aspects of the present disclosure provide methods comprising administering to a subject the RSV RNA vaccine as provided herein in a therapeutically effective amount to induce in the subject a neutralizing antibody titer against the RSV antigen delivered to the subject. In some embodiments, provided are methods comprising administering to a subject the RSV RNA vaccine as provided herein in a therapeutically effective amount to induct in the subject a RSV F neutralizing antibody titer.

In some embodiments, the vaccine is administered intramuscularly.

Also provided herein, in some aspects, are methods comprising intramuscularly administering to a subject a respiratory syncytial virus (RSV) vaccine, comprising a ribonucleic acid (RNA) that comprises a sequence that is at least 95% identical to a sequence identified by SEQ ID NO: 21 formulated in a lipid nanoparticle, wherein an at least 5-fold neutralizing antibody titer is induced in the subject following administration of a dose of the vaccine that is at least 5-fold lower relative to a control, wherein the control is a RNA vaccine encoding a membrane-bound DS-CAV1-stabilized prefusion F protein of RSV, optionally wherein the control comprises a sequence identified by SEQ ID NO: 90 or SEQ ID NO: 92. In some embodiments, the RNA of the RSV vaccine comprise a sequence identified by SEQ ID NO: 22.

Provided herein in other aspects are methods comprising intramuscularly administering to a subject a respiratory syncytial virus (RSV) vaccine, comprising a ribonucleic acid (RNA) that comprises a sequence that is at least 95% identical to a sequence identified by SEQ ID NO: 75 formulated in a lipid nanoparticle, wherein an at least 5-fold neutralizing antibody titer is induced in the subject following administration of a dose of the vaccine that is at least 5-fold lower relative to a control, wherein the control is a RNA vaccine encoding a membrane-bound DS-CAV1-stabilized prefusion F protein of RSV. In some embodiments, the RNA of the RSV vaccine comprises a sequence identified by SEQ ID NO: 75. In some embodiments, the control RNA RSV vaccine encodes a membrane-bound DS-CAV1-stabilized prefusion F protein of RSV and the sequence of the control comprises a sequence identified by SEQ ID NO: 90 or SEQ ID NO: 92.

In some aspects, the present disclosure provides an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 22. In some embodiments, the ORF shares at least 96% identity with SEQ ID NO: 22. In other embodiments, the ORF shares at least 97% identity with SEQ ID NO: 22. In some embodiments, the ORF shares at least 98% identity with SEQ ID NO: 22. In some embodiments, the ORF shares at least 99% identity SEQ ID NO: 22. In some embodiments, the ORF comprises the sequence of SEQ ID NO: 22.

In some aspects, the present disclosure provides an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 3. In some embodiments, the ORF shares at least 96% identity with SEQ ID NO: 3. In other embodiments, the ORF shares at least 97% identity with SEQ ID NO: 3. In some embodiments, the ORF shares at least 98% identity with SEQ ID NO: 3. In some embodiments, the ORF shares at least 99% identity SEQ ID NO: 22. In some embodiments, the ORF comprises the sequence of SEQ ID NO: 3.

In some aspects, the present disclosure provides an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 52. In some embodiments, the ORF shares at least 96% identity with SEQ ID NO: 52. In other embodiments, the ORF shares at least 97% identity with SEQ ID NO: 52. In some embodiments, the ORF shares at least 98% identity with SEQ ID NO: 52. In some embodiments, the ORF shares at least 99% identity SEQ ID NO: 52. In some embodiments, the ORF comprises the sequence of SEQ ID NO: 52.

In some aspects, the present disclosure provides an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 54. In some embodiments, the ORF shares at least 96% identity with SEQ ID NO: 54. In other embodiments, the ORF shares at least 97% identity with SEQ ID NO: 54. In some embodiments, the ORF shares at least 98% identity with SEQ ID NO: 54. In some embodiments, the ORF shares at least 99% identity SEQ ID NO: 54. In some embodiments, the ORF comprises the sequence of SEQ ID NO: 54.

In some aspects, the present disclosure provides an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 56. In some embodiments, the ORF shares at least 96% identity with SEQ ID NO: 56. In other embodiments, the ORF shares at least 97% identity with SEQ ID NO: 56. In some embodiments, the ORF shares at least 98% identity with SEQ ID NO: 56. In some embodiments, the ORF shares at least 99% identity SEQ ID NO: 56. In some embodiments, the ORF comprises the sequence of SEQ ID NO: 56.

In some aspects, the present disclosure provides an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 58. In some embodiments, the ORF shares at least 96% identity with SEQ ID NO: 58. In other embodiments, the ORF shares at least 97% identity with SEQ ID NO: 58. In some embodiments, the ORF shares at least 98% identity with SEQ ID NO: 58. In some embodiments, the ORF shares at least 99% identity SEQ ID NO: 58. In some embodiments, the ORF comprises the sequence of SEQ ID NO: 58.

In some aspects, the present disclosure provides an immunogenic composition (e.g., vaccine) comprising a RNA (e.g., mRNA) that comprises an ORF encoding an RSV antigen, wherein the RSV antigen comprises (or consists of, or consists essentially of) SEQ ID NO: 5 (with or without a signal sequence MELLILKANAITTILTAVTFCFASG (SEQ ID NO: 100)).

In some embodiments, the immunogenic composition is formulated in a lipid nanoparticle. In some embodiments, the lipid nanoparticle comprises a molar ratio of 20-60% ionizable cationic lipid, 5-25% non-cationic lipid, 25-55% sterol, and 0.5-15% PEG-modified lipid. In some embodiments, the lipid nanoparticle comprises (13Z,16Z)—N,N-dimethyl-2-nonylhenicosa-12,15-dien-1-amine, cholesterol, DSPC, and PEG-2000 DMG. In some embodiments, the lipid nanoparticle comprises 55-65 mole % (13Z,16Z)—N,N-dimethyl-2-nonylhenicosa-12,15-dien-1-amine, 25-35 mole % cholesterol, 5-15 mole % DSPC, and 1-5 mole % PEG-2000 DMG. In some embodiments, the lipid nanoparticle comprises 58 mole % (13Z,16Z)—N,N-dimethyl-2-nonylhenicosa-12,15-dien-1-amine, 30 mole % cholesterol, 10 mole % DSPC, and 2 mole % PEG-2000 DMG.

Thus, in some aspects, immunogenic compositions of the present disclosure comprise a RNA (e.g., mRNA) that comprises an ORF that comprises (or consists of, or consists essentially of) a sequence that shares at least 95% identity with a sequence of SEQ ID NO: 22, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some aspects, immunogenic compositions of the present disclosure comprise a RNA (e.g., mRNA) that comprises an ORF that comprises (or consists of, or consists essentially of) a sequence that shares at least 95% identity with a sequence of SEQ ID NO: 3, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some aspects, immunogenic compositions of the present disclosure comprise a RNA (e.g., mRNA) that comprises an ORF that comprises (or consists of, or consists essentially of) a sequence that shares at least 95% identity with a sequence of SEQ ID NO: 52, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some aspects, immunogenic compositions of the present disclosure comprise a RNA (e.g., mRNA) that comprises an ORF that comprises (or consists of, or consists essentially of) a sequence that shares at least 95% identity with a sequence of SEQ ID NO: 54, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some aspects, immunogenic compositions of the present disclosure comprise a RNA (e.g., mRNA) that comprises an ORF that comprises (or consists of, or consists essentially of) a sequence that shares at least 95% identity with a sequence of SEQ ID NO: 56, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some aspects, immunogenic compositions of the present disclosure comprise a RNA (e.g., mRNA) that comprises an ORF that comprises (or consists of, or consists essentially of) a sequence that shares at least 95% identity with a sequence of SEQ ID NO: 58, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some embodiments of the immunogenic compositions of the present disclosure, the RNA further comprises a 5′ cap. In some embodiments, the 5′ cap comprises 7mG(5′)ppp(5′)NlmpNp.

In some embodiments of the immunogenic compositions of the present disclosure, the RNA further comprises a 5′ UTR and/or 3′ UTR. In some embodiments, the RNA further comprises a 5′ UTR comprising a sequence of SEQ ID NO: 2. In some embodiments, the RNA further comprises a 3′ UTR comprising a sequence of SEQ ID NO: 4. In some embodiments, the RNA further comprises a 5′ UTR comprising a sequence of SEQ ID NO: 2 and a 3′ UTR comprising a sequence of SEQ ID NO: 4.

In some embodiments, the RNA further comprises a polyA tail. In some embodiments, the polyA tail has a length of 100 nucleotides. In some embodiments, the polyA tail has at least 50 nucleotides. In some embodiments, the polyA tail has at least 60 nucleotides. In some embodiments, the polyA tail has at least 70 nucleotides. In some embodiments, the polyA tail has at least 80 nucleotides. In some embodiments, the polyA tail has at least 90 nucleotides. In some embodiments, the polyA tail has at least 100 nucleotides. In some embodiments, the polyA tail has 100 nucleotides.

In some embodiments of the immunogenic compositions of the present disclosure, the RNA is chemically modified. In some embodiments, at least 80%, at least 90%, 80%-100%, 90%-100%, or 100% of the uracil residues of the RNA comprise a chemical modification. In some embodiments, the chemical modification is 1-methylpseudouridine.

In some aspects, an immunogenic composition comprises a RNA (e.g., mRNA) that comprises an ORF that comprises (or consists of, or consists essentially of) a sequence that shares at least 95% identity with a sequence of SEQ ID NO: 22, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

Thus, in some aspects, an immunogenic composition comprises a RNA (e.g., mRNA) that comprises an ORF that comprises (or consists of, or consists essentially of) a sequence that shares at least 95% identity with a sequence of SEQ ID NO: 22, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail. In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, an immunogenic composition comprises a RNA (e.g., mRNA) that comprises an ORF that comprises (or consists of, or consists essentially of) a sequence that shares at least 95% identity with a sequence of SEQ ID NO: 3, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

Thus, in some aspects, an immunogenic composition comprises a RNA (e.g., mRNA) that comprises an ORF that comprises (or consists of, or consists essentially of) a sequence that shares at least 95% identity with a sequence of SEQ ID NO: 3, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail. In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, an immunogenic composition comprises a RNA (e.g., mRNA) that comprises an ORF that comprises (or consists of, or consists essentially of) a sequence that shares at least 95% identity with a sequence of SEQ ID NO: 52, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

Thus, in some aspects, an immunogenic composition comprises a RNA (e.g., mRNA) that comprises an ORF that comprises (or consists of, or consists essentially of) a sequence that shares at least 95% identity with a sequence of SEQ ID NO: 52, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail. In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, an immunogenic composition comprises a RNA (e.g., mRNA) that comprises an ORF that comprises (or consists of, or consists essentially of) a sequence that shares at least 95% identity with a sequence of SEQ ID NO: 54, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

Thus, in some aspects, an immunogenic composition comprises a RNA (e.g., mRNA) that comprises an ORF that comprises (or consists of, or consists essentially of) a sequence that shares at least 95% identity with a sequence of SEQ ID NO: 54, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail. In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, an immunogenic composition comprises a RNA (e.g., mRNA) that comprises an ORF that comprises (or consists of, or consists essentially of) a sequence that shares at least 95% identity with a sequence of SEQ ID NO: 56, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

Thus, in some aspects, an immunogenic composition comprises a RNA (e.g., mRNA) that comprises an ORF that comprises (or consists of, or consists essentially of) a sequence that shares at least 95% identity with a sequence of SEQ ID NO: 56, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail. In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, an immunogenic composition comprises a RNA (e.g., mRNA) that comprises an ORF that comprises (or consists of, or consists essentially of) a sequence that shares at least 95% identity with a sequence of SEQ ID NO: 58, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

Thus, in some aspects, an immunogenic composition comprises a RNA (e.g., mRNA) that comprises an ORF that comprises (or consists of, or consists essentially of) a sequence that shares at least 95% identity with a sequence of SEQ ID NO: 58, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail. In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the present disclosure provides an immunogenic composition (e.g., vaccine) comprising a RNA (e.g., mRNA) that comprises (or consists of, or consists essentially of) a sequence that shares at least 95% identity with SEQ ID NO: 21. In some embodiments, the RNA comprises a sequence that shares at least 96% identity with SEQ ID NO: 21. In some embodiments, the RNA comprises a sequence that shares at least 97% identity with SEQ ID NO: 21. In other embodiments, the RNA comprises a sequence that shares at least 98% identity with SEQ ID NO: 21. In some embodiments, the RNA comprises a sequence that shares at least 99% identity with SEQ ID NO: 21. In some embodiments, the RNA comprises the sequence of SEQ ID NO: 21. In some embodiments, the RNA consists of the sequence of SEQ ID NO: 21. In some embodiments, the RNA consists essentially of the sequence of SEQ ID NO: 21.

In some aspects, the present disclosure provides an immunogenic composition (e.g., vaccine) comprising a RNA (e.g., mRNA) that comprises (or consists of, or consists essentially of) a sequence that shares at least 95% identity with SEQ ID NO: 1. In some embodiments, the RNA comprises a sequence that shares at least 96% identity with SEQ ID NO: 1. In some embodiments, the RNA comprises a sequence that shares at least 97% identity with SEQ ID NO: 1. In other embodiments, the RNA comprises a sequence that shares at least 98% identity with SEQ ID NO: 1. In some embodiments, the RNA comprises a sequence that shares at least 99% identity with SEQ ID NO: 1. In some embodiments, the RNA comprises the sequence of SEQ ID NO: 1. In some embodiments, the RNA consists of the sequence of SEQ ID NO: 1. In some embodiments, the RNA consists essentially of the sequence of SEQ ID NO: 1.

In some aspects, the present disclosure provides an immunogenic composition (e.g., vaccine) comprising a RNA (e.g., mRNA) that comprises (or consists of, or consists essentially of) a sequence that shares at least 95% identity with SEQ ID NO: 51. In some embodiments, the RNA comprises a sequence that shares at least 96% identity with SEQ ID NO: 51. In some embodiments, the RNA comprises a sequence that shares at least 97% identity with SEQ ID NO: 51. In other embodiments, the RNA comprises a sequence that shares at least 98% identity with SEQ ID NO: 51. In some embodiments, the RNA comprises a sequence that shares at least 99% identity with SEQ ID NO: 51. In some embodiments, the RNA comprises the sequence of SEQ ID NO: 51. In some embodiments, the RNA consists of the sequence of SEQ ID NO: 51. In some embodiments, the RNA consists essentially of the sequence of SEQ ID NO: 51.

In some aspects, the present disclosure provides an immunogenic composition (e.g., vaccine) comprising a RNA (e.g., mRNA) that comprises (or consists of, or consists essentially of) a sequence that shares at least 95% identity with SEQ ID NO: 53. In some embodiments, the RNA comprises a sequence that shares at least 96% identity with SEQ ID NO: 53. In some embodiments, the RNA comprises a sequence that shares at least 97% identity with SEQ ID NO: 53. In other embodiments, the RNA comprises a sequence that shares at least 98% identity with SEQ ID NO: 53. In some embodiments, the RNA comprises a sequence that shares at least 99% identity with SEQ ID NO: 53. In some embodiments, the RNA comprises the sequence of SEQ ID NO: 53. In some embodiments, the RNA consists of the sequence of SEQ ID NO: 53. In some embodiments, the RNA consists essentially of the sequence of SEQ ID NO: 53.

In some aspects, the present disclosure provides an immunogenic composition (e.g., vaccine) comprising a RNA (e.g., mRNA) that comprises (or consists of, or consists essentially of) a sequence that shares at least 95% identity with SEQ ID NO: 55. In some embodiments, the RNA comprises a sequence that shares at least 96% identity with SEQ ID NO: 55. In some embodiments, the RNA comprises a sequence that shares at least 97% identity with SEQ ID NO: 55. In other embodiments, the RNA comprises a sequence that shares at least 98% identity with SEQ ID NO: 55. In some embodiments, the RNA comprises a sequence that shares at least 99% identity with SEQ ID NO: 55. In some embodiments, the RNA comprises the sequence of SEQ ID NO: 55. In some embodiments, the RNA consists of the sequence of SEQ ID NO: 55. In some embodiments, the RNA consists essentially of the sequence of SEQ ID NO: 55.

In some aspects, the present disclosure provides an immunogenic composition (e.g., vaccine) comprising a RNA (e.g., mRNA) that comprises (or consists of, or consists essentially of) a sequence that shares at least 95% identity with SEQ ID NO: 57. In some embodiments, the RNA comprises a sequence that shares at least 96% identity with SEQ ID NO: 57. In some embodiments, the RNA comprises a sequence that shares at least 97% identity with SEQ ID NO: 57. In other embodiments, the RNA comprises a sequence that shares at least 98% identity with SEQ ID NO: 57. In some embodiments, the RNA comprises a sequence that shares at least 99% identity with SEQ ID NO: 57. In some embodiments, the RNA comprises the sequence of SEQ ID NO: 57. In some embodiments, the RNA consists of the sequence of SEQ ID NO: 57. In some embodiments, the RNA consists essentially of the sequence of SEQ ID NO: 57.

In some aspects, the present disclosure provides an immunogenic composition (e.g., vaccine) comprising a mRNA that comprises (or consists of, or consists essentially of) the sequence of SEQ ID NO: 21 formulated in a lipid nanoparticle comprising 58 mole % (13Z,16Z)—N,N-dimethyl-2-nonylhenicosa-12,15-dien-1-amine, 30 mole % cholesterol, 10 mole % DSPC, and 2 mole % PEG-2000 DMG, wherein the mRNA further comprises a 5′ 7mG(5′)ppp(5′)NlmpNp cap, and wherein at least 80% of the uracil of the mRNA are 1-methylpseudouridine.

Also provided herein are methods of inducing in a subject an immune response to RSV, the methods comprising administering to the subject any of the immunogenic compositions (e.g., vaccines) described above.

Thus, in some aspects, the present disclosure provides methods that comprise delivering to a subject (e.g., via intramuscular administration) an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 22. In some embodiments, the ORF shares at least 96% identity with SEQ ID NO: 22. In other embodiments, the ORF shares at least 97% identity with SEQ ID NO: 22. In some embodiments, the ORF shares at least 98% identity with SEQ ID NO: 22. In some embodiments, the ORF shares at least 99% identity SEQ ID NO: 22. In some embodiments, the ORF comprises the sequence of SEQ ID NO: 22.

In some aspects, the present disclosure provides methods that comprise delivering to a subject (e.g., via intramuscular administration) an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 3. In some embodiments, the ORF shares at least 96% identity with SEQ ID NO: 3. In other embodiments, the ORF shares at least 97% identity with SEQ ID NO: 3. In some embodiments, the ORF shares at least 98% identity with SEQ ID NO: 3. In some embodiments, the ORF shares at least 99% identity SEQ ID NO: 3. In some embodiments, the ORF comprises the sequence of SEQ ID NO: 3.

In some aspects, the present disclosure provides methods that comprise delivering to a subject (e.g., via intramuscular administration) an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 52. In some embodiments, the ORF shares at least 96% identity with SEQ ID NO: 52. In other embodiments, the ORF shares at least 97% identity with SEQ ID NO: 52. In some embodiments, the ORF shares at least 98% identity with SEQ ID NO: 52. In some embodiments, the ORF shares at least 99% identity SEQ ID NO: 52. In some embodiments, the ORF comprises the sequence of SEQ ID NO: 52.

In some aspects, the present disclosure provides methods that comprise delivering to a subject (e.g., via intramuscular administration) an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 54. In some embodiments, the ORF shares at least 96% identity with SEQ ID NO: 54. In other embodiments, the ORF shares at least 97% identity with SEQ ID NO: 54. In some embodiments, the ORF shares at least 98% identity with SEQ ID NO: 54. In some embodiments, the ORF shares at least 99% identity SEQ ID NO: 54. In some embodiments, the ORF comprises the sequence of SEQ ID NO: 54.

In some aspects, the present disclosure provides methods that comprise delivering to a subject (e.g., via intramuscular administration) an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 56. In some embodiments, the ORF shares at least 96% identity with SEQ ID NO: 56. In other embodiments, the ORF shares at least 97% identity with SEQ ID NO: 56. In some embodiments, the ORF shares at least 98% identity with SEQ ID NO: 56. In some embodiments, the ORF shares at least 99% identity SEQ ID NO: 56. In some embodiments, the ORF comprises the sequence of SEQ ID NO: 56.

In some aspects, the present disclosure provides methods that comprise delivering to a subject (e.g., via intramuscular administration) an immunogenic composition (e.g., vaccine) comprising a RNA (e.g., mRNA) that comprises an ORF encoding an RSV antigen, wherein the ORF comprises (or consists of, or consists essentially of) a sequence that shares at least 95% identity with SEQ ID NO: 58. In some embodiments, the ORF shares at least 96% identity with SEQ ID NO: 58. In other embodiments, the ORF shares at least 97% identity with SEQ ID NO: 58. In some embodiments, the ORF shares at least 98% identity with SEQ ID NO: 58. In some embodiments, the ORF shares at least 99% identity SEQ ID NO: 58. In some embodiments, the ORF comprises the sequence of SEQ ID NO: 58.

In some aspects, the present disclosure provides methods that comprise delivering to a subject (e.g., via intramuscular administration) an immunogenic composition (e.g., vaccine) comprising a RNA (e.g., mRNA) that comprises an ORF encoding an RSV antigen, wherein the RSV antigen comprises (or consists of, or consists essentially of) SEQ ID NO: 5 (with or without a signal sequence MELLILKANAITTILTAVTFCFASG (SEQ ID NO: 100). In some embodiments, the immunogenic composition used in the methods is formulated in a lipid nanoparticle. In some embodiments, the lipid nanoparticle comprises a molar ratio of 20-60% ionizable cationic lipid, 5-25% non-cationic lipid, 25-55% sterol, and 0.5-15% PEG-modified lipid. In some embodiments, the lipid nanoparticle comprises (13Z,16Z)—N,N-dimethyl-2-nonylhenicosa-12,15-dien-1-amine, cholesterol, DSPC, and PEG-2000 DMG. In some embodiments, the lipid nanoparticle comprises 55-65 mole % (13Z,16Z)—N,N-dimethyl-2-nonylhenicosa-12,15-dien-1-amine, 25-35 mole % cholesterol, 5-15 mole % DSPC, and 1-5 mole % PEG-2000 DMG. In some embodiments, the lipid nanoparticle comprises 58 mole % (13Z,16Z)—N,N-dimethyl-2-nonylhenicosa-12,15-dien-1-amine, 30 mole % cholesterol, 10 mole % DSPC, and 2 mole % PEG-2000 DMG.

Thus, in some aspects, the methods comprise delivering to a subject (e.g., via intramuscular administration) an immunogenic composition that comprises a RNA (e.g., mRNA) that comprises an ORF that comprises (or consists of, or consists essentially of) a sequence that shares at least 95% identity with a sequence of SEQ ID NO: 22, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG. In some embodiments, the RNA further comprises a 5′ cap. In some embodiments, the 5′ cap comprises 7mG(5′)ppp(5′)NlmpNp. In some embodiments, the RNA of the immunogenic composition used in the methods provided herein further comprises a 5′ UTR and/or 3′ UTR. In some embodiments, the RNA further comprises a 5′ UTR comprising a sequence of SEQ ID NO: 2. In some embodiments, the RNA further comprises a 3′ UTR comprising a sequence of SEQ ID NO: 4. In some embodiments, the RNA further comprises a 5′ UTR comprising a sequence of SEQ ID NO: 2 and a 3′ UTR comprising a sequence of SEQ ID NO: 4.

In some aspects, the methods comprise delivering to a subject (e.g., via intramuscular administration) an immunogenic composition that comprises a RNA (e.g., mRNA) that comprises an ORF that comprises (or consists of, or consists essentially of) a sequence that shares at least 95% identity with a sequence of SEQ ID NO: 3, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some aspects, the methods comprise delivering to a subject (e.g., via intramuscular administration) an immunogenic composition that comprises a RNA (e.g., mRNA) that comprises an ORF that comprises (or consists of, or consists essentially of) a sequence that shares at least 95% identity with a sequence of SEQ ID NO: 52, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some aspects, the methods comprise delivering to a subject (e.g., via intramuscular administration) an immunogenic composition that comprises a RNA (e.g., mRNA) that comprises an ORF that comprises (or consists of, or consists essentially of) a sequence that shares at least 95% identity with a sequence of SEQ ID NO: 54, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some aspects, the methods comprise delivering to a subject (e.g., via intramuscular administration) an immunogenic composition that comprises a RNA (e.g., mRNA) that comprises an ORF that comprises (or consists of, or consists essentially of) a sequence that shares at least 95% identity with a sequence of SEQ ID NO: 56, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG.

In some aspects, the methods comprise delivering to a subject (e.g., via intramuscular administration) an immunogenic composition that comprises a RNA (e.g., mRNA) that comprises an ORF that comprises (or consists of, or consists essentially of) a sequence that shares at least 95% identity with a sequence of SEQ ID NO: 58, wherein the RNA is formulated in a lipid nanoparticle that comprises (13Z,16Z)—N,N-dimethyl-2-nonylhenicosa-12,15-dien-1-amine, cholesterol, DSPC, and PEG-2000 DMG. In some embodiments, the RNA of the immunogenic composition used in the methods provided herein further comprises a polyA tail. In some embodiments, the polyA tail has a length of 100 nucleotides.

In some embodiments, the polyA tail has at least 50 nucleotides. In some embodiments, the polyA tail has at least 60 nucleotides. In some embodiments, the polyA tail has at least 70 nucleotides. In some embodiments, the polyA tail has at least 80 nucleotides. In some embodiments, the polyA tail has at least 90 nucleotides. In some embodiments, the polyA tail has at least 100 nucleotides. In some embodiments, the polyA tail has 100 nucleotides.

In some embodiments, the RNA of the immunogenic composition used in the methods provided herein is chemically modified. In some embodiments, at least 80%, at least 90%, 80%-100%, 90%-100%, or 100% of the uracil residues of the RNA comprise a chemical modification. In some embodiments, the chemical modification is 1-methylpseudouridine.

In some aspects, the RNA (e.g., mRNA) of the immunogenic composition used in the methods provided herein comprises an ORF that comprises (or consists of, or consists essentially of) a sequence that shares at least 95% identity with a sequence of SEQ ID NO: 22, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the RNA (e.g., mRNA) of the immunogenic composition used in the methods provided herein comprises an ORF that comprises (or consists of, or consists essentially of) a sequence that shares at least 95% identity with a sequence of SEQ ID NO: 3, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the RNA (e.g., mRNA) of the immunogenic composition used in the methods provided herein comprises an ORF that comprises (or consists of, or consists essentially of) a sequence that shares at least 95% identity with a sequence of SEQ ID NO: 52, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the RNA (e.g., mRNA) of the immunogenic composition used in the methods provided herein comprises an ORF that comprises (or consists of, or consists essentially of) a sequence that shares at least 95% identity with a sequence of SEQ ID NO: 54, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the RNA (e.g., mRNA) of the immunogenic composition used in the methods provided herein comprises an ORF that comprises (or consists of, or consists essentially of) a sequence that shares at least 95% identity with a sequence of SEQ ID NO: 56, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the RNA (e.g., mRNA) of the immunogenic composition used in the methods provided herein comprises an ORF that comprises (or consists of, or consists essentially of) a sequence that shares at least 95% identity with a sequence of SEQ ID NO: 58, wherein the RNA further comprise a 5′ cap, a 5′ UTR, a 3′ UTR and a polyA tail. In some embodiments, RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the methods of the present disclosure provide delivering to a subject (e.g., via intramuscular injection) an immunogenic composition comprising a RNA (e.g., mRNA) that comprises an ORF that comprises (or consists of, or consists essentially of) a sequence that shares at least 95% identity with a sequence of SEQ ID NO: 22, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail.

In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the methods of the present disclosure provide delivering to a subject (e.g., via intramuscular injection) an immunogenic composition comprising a RNA (e.g., mRNA) that comprises an ORF that comprises (or consists of, or consists essentially of) a sequence that shares at least 95% identity with a sequence of SEQ ID NO: 3, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail.

In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the methods of the present disclosure provide delivering to a subject (e.g., via intramuscular injection) an immunogenic composition comprising a RNA (e.g., mRNA) that comprises an ORF that comprises (or consists of, or consists essentially of) a sequence that shares at least 95% identity with a sequence of SEQ ID NO: 52, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail.

In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the methods of the present disclosure provide delivering to a subject (e.g., via intramuscular injection) an immunogenic composition comprising a RNA (e.g., mRNA) that comprises an ORF that comprises (or consists of, or consists essentially of) a sequence that shares at least 95% identity with a sequence of SEQ ID NO: 54, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail. In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the methods of the present disclosure provide delivering to a subject (e.g., via intramuscular injection) an immunogenic composition comprising a RNA (e.g., mRNA) that comprises an ORF that comprises (or consists of, or consists essentially of) a sequence that shares at least 95% identity with a sequence of SEQ ID NO: 56, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail.

In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the methods of the present disclosure provide delivering to a subject (e.g., via intramuscular injection) an immunogenic composition comprising a RNA (e.g., mRNA) that comprises an ORF that comprises (or consists of, or consists essentially of) a sequence that shares at least 95% identity with a sequence of SEQ ID NO: 58, wherein the RNA further comprise a 5′ 7mG(5′)ppp(5′)NlmpNp cap, a 5′ UTR comprising the sequence of SEQ ID NO: 2, and a 3′ UTR comprising the sequence of SEQ ID NO: 4, and a polyA tail. In some embodiments, the polyA tail has a length of at least 100 nucleotides. In some embodiments, the RNA is formulated in a lipid nanoparticle. In some embodiments, the lipid nanoparticle comprises (13Z,16Z)—N,N-dimethyl-2-nonylhenicosa-12,15-dien-1-amine, cholesterol, DSPC, and PEG-2000 DMG.

In some aspects, the present disclosure provides methods comprising delivering to a subject (e.g., via intramuscular injection) an immunogenic composition (e.g., vaccine) comprising a RNA (e.g., mRNA) that comprises (or consists of, or consists essentially of) a sequence that shares at least 95% identity with SEQ ID NO: 21. In some embodiments, the RNA comprises a sequence that shares at least 96% identity with SEQ ID NO: 21. In some embodiments, the RNA comprises a sequence that shares at least 97% identity with SEQ ID NO: 21. In other embodiments, the RNA comprises a sequence that shares at least 98% identity with SEQ ID NO: 21. In some embodiments, the RNA comprises a sequence that shares at least 99% identity with SEQ ID NO: 21. In some embodiments, the RNA comprises the sequence of SEQ ID NO: 21. In some embodiments, the RNA consists of the sequence of SEQ ID NO: 21. In some embodiments, the RNA consists essentially of the sequence of SEQ ID NO: 21.

In some aspects, the present disclosure provides methods comprising delivering to a subject (e.g., via intramuscular injection) an immunogenic composition (e.g., vaccine) comprising a RNA (e.g., mRNA) that comprises (or consists of, or consists essentially of) a sequence that shares at least 95% identity with SEQ ID NO: 1. In some embodiments, the RNA comprises a sequence that shares at least 96% identity with SEQ ID NO: 1. In some embodiments, the RNA comprises a sequence that shares at least 97% identity with SEQ ID NO: 1. In other embodiments, the RNA comprises a sequence that shares at least 98% identity with SEQ ID NO: 1. In some embodiments, the RNA comprises a sequence that shares at least 99% identity with SEQ ID NO: 1. In some embodiments, the RNA comprises the sequence of SEQ ID NO: 1. In some embodiments, the RNA consists of the sequence of SEQ ID NO: 1. In some embodiments, the RNA consists essentially of the sequence of SEQ ID NO: 1.

In some aspects, the present disclosure provides methods comprising delivering to a subject (e.g., via intramuscular injection) an immunogenic composition (e.g., vaccine) comprising a RNA (e.g., mRNA) that comprises (or consists of, or consists essentially of) a sequence that shares at least 95% identity with SEQ ID NO: 51. In some embodiments, the RNA comprises a sequence that shares at least 96% identity with SEQ ID NO: 51. In some embodiments, the RNA comprises a sequence that shares at least 97% identity with SEQ ID NO: 51. In other embodiments, the RNA comprises a sequence that shares at least 98% identity with SEQ ID NO: 51. In some embodiments, the RNA comprises a sequence that shares at least 99% identity with SEQ ID NO: 51. In some embodiments, the RNA comprises the sequence of SEQ ID NO: 51. In some embodiments, the RNA consists of the sequence of SEQ ID NO: 51. In some embodiments, the RNA consists essentially of the sequence of SEQ ID NO: 51.

In some aspects, the present disclosure provides methods comprising delivering to a subject (e.g., via intramuscular injection) an immunogenic composition (e.g., vaccine) comprising a RNA (e.g., mRNA) that comprises (or consists of, or consists essentially of) a sequence that shares at least 95% identity with SEQ ID NO: 53. In some embodiments, the RNA comprises a sequence that shares at least 96% identity with SEQ ID NO: 53. In some embodiments, the RNA comprises a sequence that shares at least 97% identity with SEQ ID NO: 53. In other embodiments, the RNA comprises a sequence that shares at least 98% identity with SEQ ID NO: 53. In some embodiments, the RNA comprises a sequence that shares at least 99% identity with SEQ ID NO: 53. In some embodiments, the RNA comprises the sequence of SEQ ID NO: 53. In some embodiments, the RNA consists of the sequence of SEQ ID NO: 53. In some embodiments, the RNA consists essentially of the sequence of SEQ ID NO: 53.

In some aspects, the present disclosure provides methods comprising delivering to a subject (e.g., via intramuscular injection) an immunogenic composition (e.g., vaccine) comprising a RNA (e.g., mRNA) that comprises (or consists of, or consists essentially of) a sequence that shares at least 95% identity with SEQ ID NO: 55. In some embodiments, the RNA comprises a sequence that shares at least 96% identity with SEQ ID NO: 55. In some embodiments, the RNA comprises a sequence that shares at least 97% identity with SEQ ID NO: 55. In other embodiments, the RNA comprises a sequence that shares at least 98% identity with SEQ ID NO: 55. In some embodiments, the RNA comprises a sequence that shares at least 99% identity with SEQ ID NO: 55. In some embodiments, the RNA comprises the sequence of SEQ ID NO: 55. In some embodiments, the RNA consists of the sequence of SEQ ID NO: 55. In some embodiments, the RNA consists essentially of the sequence of SEQ ID NO: 55.

In some aspects, the present disclosure provides methods comprising delivering to a subject (e.g., via intramuscular injection) an immunogenic composition (e.g., vaccine) comprising a RNA (e.g., mRNA) that comprises (or consists of, or consists essentially of) a sequence that shares at least 95% identity with SEQ ID NO: 57. In some embodiments, the RNA comprises a sequence that shares at least 96% identity with SEQ ID NO: 57. In some embodiments, the RNA comprises a sequence that shares at least 97% identity with SEQ ID NO: 57. In other embodiments, the RNA comprises a sequence that shares at least 98% identity with SEQ ID NO: 57. In some embodiments, the RNA comprises a sequence that shares at least 99% identity with SEQ ID NO: 57. In some embodiments, the RNA comprises the sequence of SEQ ID NO: 57. In some embodiments, the RNA consists of the sequence of SEQ ID NO: 57. In some embodiments, the RNA consists essentially of the sequence of SEQ ID NO: 57.

In some aspects, the present disclosure provides methods that comprise delivering to a subject (e.g., via intramuscular injection) an immunogenic composition (e.g., vaccine) comprising a mRNA that comprises (or consists of, or consists essentially of) the sequence of SEQ ID NO: 21 formulated in a lipid nanoparticle comprising 58 mole % (13Z,16Z)—N,N-dimethyl-2-nonylhenicosa-12,15-dien-1-amine, 30 mole % cholesterol, 10 mole % DSPC, and 2 mole % PEG-2000 DMG, wherein the mRNA further comprises a 5′ 7mG(5′)ppp(5′)NlmpNp cap, and wherein at least 80% of the uracil of the mRNA are 1-methylpseudouridine.

In some aspects, the present disclosure provides methods that comprise delivering to a subject (e.g., via intramuscular injection) an immunogenic composition (e.g., vaccine) comprising a mRNA that comprises (or consists of, or consists essentially of) the sequence of SEQ ID NO: 1 formulated in a lipid nanoparticle comprising 58 mole % (13Z,16Z)—N,N-dimethyl-2-nonylhenicosa-12,15-dien-1-amine, 30 mole % cholesterol, 10 mole % DSPC, and 2 mole % PEG-2000 DMG, wherein the mRNA further comprises a 5′ 7mG(5′)ppp(5′)NlmpNp cap, and wherein at least 80% of the uracil of the mRNA are 1-methylpseudouridine.

In some aspects, the present disclosure provides methods that comprise delivering to a subject (e.g., via intramuscular injection) an immunogenic composition (e.g., vaccine) comprising a mRNA that comprises (or consists of, or consists essentially of) the sequence of SEQ ID NO: 51 formulated in a lipid nanoparticle comprising 58 mole % (13Z,16Z)—N,N-dimethyl-2-nonylhenicosa-12,15-dien-1-amine, 30 mole % cholesterol, 10 mole % DSPC, and 2 mole % PEG-2000 DMG, wherein the mRNA further comprises a 5′ 7mG(5′)ppp(5′)NlmpNp cap, and wherein at least 80% of the uracil of the mRNA are 1-methylpseudouridine.

In some aspects, the present disclosure provides methods that comprise delivering to a subject (e.g., via intramuscular injection) an immunogenic composition (e.g., vaccine) comprising a mRNA that comprises (or consists of, or consists essentially of) the sequence of SEQ ID NO: 53 formulated in a lipid nanoparticle comprising 58 mole % (13Z,16Z)—N,N-dimethyl-2-nonylhenicosa-12,15-dien-1-amine, 30 mole % cholesterol, 10 mole % DSPC, and 2 mole % PEG-2000 DMG, wherein the mRNA further comprises a 5′ 7mG(5′)ppp(5′)NlmpNp cap, and wherein at least 80% of the uracil of the mRNA are 1-methylpseudouridine.

In some aspects, the present disclosure provides methods that comprise delivering to a subject (e.g., via intramuscular injection) an immunogenic composition (e.g., vaccine) comprising a mRNA that comprises (or consists of, or consists essentially of) the sequence of SEQ ID NO: 55 formulated in a lipid nanoparticle comprising 58 mole % (13Z,16Z)—N,N-dimethyl-2-nonylhenicosa-12,15-dien-1-amine, 30 mole % cholesterol, 10 mole % DSPC, and 2 mole % PEG-2000 DMG, wherein the mRNA further comprises a 5′ 7mG(5′)ppp(5′)NlmpNp cap, and wherein at least 80% of the uracil of the mRNA are 1-methylpseudouridine.

In some aspects, the present disclosure provides methods that comprise delivering to a subject (e.g., via intramuscular injection) an immunogenic composition (e.g., vaccine) comprising a mRNA that comprises (or consists of, or consists essentially of) the sequence of SEQ ID NO: 57 formulated in a lipid nanoparticle comprising 58 mole % (13Z,16Z)—N,N-dimethyl-2-nonylhenicosa-12,15-dien-1-amine, 30 mole % cholesterol, 10 mole % DSPC, and 2 mole % PEG-2000 DMG, wherein the mRNA further comprises a 5′ 7mG(5′)ppp(5′)NlmpNp cap, and wherein at least 80% of the uracil of the mRNA are 1-methylpseudouridine.

### BRIEF DESCRIPTION OF THE DRAWINGS

FIG. 1 shows a graph of serum neutralizing antibody titers (NT₅₀ individual and GMT with 95% confidence intervals) to RSV A induced in cotton rats by mRNA vaccines and control formulations.

FIG. 2 shows a graph of serum antibody competition ELISA Titers (IT₅₀ Individual and GMT with 95% confidence intervals) against D25 (site Ø), palivizumab (site II), and 4D7 (site I) measured at Day 56 (4 weeks PD2).

FIG. 3 shows graphs of RSV content in lung and nose after challenge of cotton rats with RSV A.

FIG. 4 shows serum neutralizing antibody titers (NT₅₀ Individual and GMT with 95% confidence intervals) to RSV A induced in African green monkeys by mRNA vaccines and control Formulations.

FIG. 5 shows serum antibody competition ELISA titers (IT₅₀ Individual and GMT with 95% confidence intervals) against D25 (site Ø), palivizumab (site II), and 4D7 (site I) measured at week 10 (2 weeks PD3).

FIGS. 6A-6C shows RSV content in bronchoalveolar (BAL) fluid after challenge of AGMs FIG. 6A: High dose immunization. FIG. 6B: Low dose immunization. FIG. 6C: Area under the curve.

FIG. 7 shows serum neutralizing antibody titers (NT₅₀ Individual and GMT with 95% confidence intervals) to RSV A induced in RSV experienced African green monkeys by mRNA vaccines and control formulations.

FIG. 8 shows serum antibody competition ELISA titers (IT₅₀ Individual and GMT with 95% confidence intervals) against D25 (site Ø), palivizumab (site II), and 4D7 (site I) measured at week 2 (2 weeks PD).

### DETAILED DESCRIPTION

Embodiments of the present disclosure provide highly immunogenic RNA (e.g., mRNA) compositions (e.g., vaccines) that include a (at least one) polynucleotide encoding a respiratory syncytial virus (RSV) antigen and elicit potent neutralizing antibodies. RSV is a negative-sense, single-stranded RNA virus of the genus Pneumovirinae. The virus is present in at least two antigenic subgroups, known as Group A and Group B, primarily resulting from differences in the surface G glycoproteins. Two RSV surface glycoproteins—G and F—mediate attachment with and attachment to cells of the respiratory epithelium. F surface glycoproteins mediate coalescence of neighboring cells. This results in the formation of syncytial cells. RSV is the most common cause of bronchiolitis. Most infected adults develop mild cold-like symptoms such as congestion, low-grade fever, and wheezing. Infants and small children may suffer more severe symptoms such as bronchiolitis and pneumonia. The disease may be transmitted among humans via contact with respiratory secretions.

RSV F protein is a type I fusion glycoprotein that is well conserved between clinical isolates, including between the RSV-A and RSV-B antigenic subgroups. The F protein transitions between prefusion and more stable postfusion states, thereby facilitating entry into target cells. RSV F glycoprotein is initially synthesized as an F0 precursor protein. RSV F0 folds into a trimer, which is activated by furin cleavage into the mature prefusion protein comprising F1 and F2 subunits (Bolt, et al., Virus Res., 68:25, 2000). RSV F protein stabilized in the prefusion conformation produce a greater neutralizing immune response in animal models than that observed with RSV F protein stabilized in the post fusion conformation (McLellan et al., Science, 342: 592-598, 2013). As such, stabilized prefusion RSV F proteins are good candidates for inclusion in an RSV vaccine. Soluble RSV ectodomains stabilized in the prefusion conformation have previously been generated, including the DS-CAV1 substitutions. “DS-CAV1” and “DS-CAV1 substitutions,” each as used herein, refers to genetic modifications to the RSV F protein, which contains the “DS” substitutions 155C and 290C so as to introduce a non-native disulfide bond between cysteines introduced by the substitutions (such as S155C and S290C substitutions) and the “CAV1” substitutions, which include 190F and 207L cavity filling amino acid substitutions (such as S190F and V207L). mVRC-1 (e.g., SEQ ID NO: 21), used in some of the immunogenic compositions of the present disclosure, as compared to DS-CAV1, includes an additional stabilizing disulfide bond (resulting from the introduction of the amino acid substitutions A149C and Y458C) and is engineered so that the F₂ and F₁ subunits are genetically linked, with the fusion peptide and p27 region deleted. See, WO 2014/160463A1 and WO 2017/172890A1, the contents of each of which are hereby incorporated by reference.

It has been previously shown that the prefusion stabilized RSV F construct, DS-CAV1, undergoes conformational changes and forms intermediate structures upon long-term storage at 4° C. (Flynn J A et al., PLoS ONE 2016; 11(10): e0164789). Long term stability at 4° C. or higher is a desirable attribute for a RSV F subunit vaccine antigen. Described herein are additional structure-based modifications to further improve the stability of the RSV F trimer in the prefusion conformation. Such constructs have increased stability at 4° C. as compared to DS-CAV1 while retaining immunogenicity. “RSV Fusion Protein” or “RSV F protein”, each as used herein, refers to an RSV envelope glycoprotein that facilitates fusion of viral and cellular membranes. In nature, the RSV F protein is synthesized into a single polypeptide precursor designated F0, which includes a signal peptide that directs localization to the endoplasmic reticulum, where the signal peptide is cleaved. The remaining F0 residues oligomerize to form a trimer and are proteolytically processed by a protease at two conserved furin cleavage sequences to generate two disulfide linked fragments, F1 and F2. In nature, three F2-F1 peptides oligomerize into a trimer to form the mature F protein, which adopts a prefusion conformation that is metastable and can undergo a conformation change to a postfusion conformation.

The genome of RSV encodes at least three surface glycoproteins, including F, G, and SH, four nucleocapsid proteins, including L, P, N, and M2, and one matrix protein, M. Glycoprotein F directs viral penetration by fusion between the virion and the host membrane. Glycoprotein G is a type II transmembrane glycoprotein and is the major attachment protein. SH is a short integral membrane protein. Matrix protein M is found in the inner layer of the lipid bilayer and assists virion formation. Nucleocapsid proteins L, P, N, and M2 modulate replication and transcription of the RSV genome. It is thought that glycoprotein G tethers and stabilizes the virus particle at the surface of bronchial epithelial cells, while glycoprotein F interacts with cellular glycosaminoglycans to mediate fusion and delivery of the RSV virion contents into the host cell (Krzyzaniak M A et al. PLoS Pathog 2013; 9(4)).

The present disclosure is not limited by a particular strain of RSV. The strain of RSV used in a vaccine may be any strain of RSV.

The RSV RNA vaccines described herein are superior to current vaccines in several ways. For example, the lipid nanoparticle (LNP) delivery system used herein increases the efficacy of RNA vaccines in comparison to other formulations, including a protamine-based approach described in the literature. The use of this LNP delivery system enables the effective delivery of chemically-modified RNA vaccines or unmodified RNA vaccines, without requiring additional adjuvant to produce a therapeutic result (e.g., production neutralizing antibody titer). In some embodiments, the RSV RNA vaccines disclosed herein are superior to conventional vaccines by a factor of at least 10 fold, 20, fold, 40, fold, 50 fold, 100 fold, 500 fold, or 1,000 fold when administered intramuscularly (IM) or intradermally (ID). These results can be achieved even when significantly lower doses of the RNA (e.g., mRNA) are administered in comparison with RNA doses used in other classes of lipid based formulations.

Although attempts have been made to produce functional RNA vaccines, including mRNA vaccines and self-replicating RNA vaccines, the therapeutic efficacy of these RNA vaccines have not yet been fully established. Quite surprisingly, the inventors have discovered, according to aspects of the present disclosure, a class of formulations for delivering mRNA vaccines in vivo that results in significantly enhanced, and in many respects synergistic, immune responses including enhanced antigen generation and functional antibody production with neutralization capability. These results can be achieved even when significantly lower doses of the mRNA are administered in comparison with mRNA doses used in other classes of lipid based formulations. The formulations of the present disclosure have demonstrated significant unexpected in vivo immune responses sufficient to establish the efficacy of functional mRNA vaccines as prophylactic and therapeutic agents. Additionally, self-replicating RNA vaccines rely on viral replication pathways to deliver enough RNA to a cell to produce an immunogenic response. The formulations of the present disclosure do not require viral replication to produce enough protein to result in a strong immune response. Thus, the mRNA of the present disclosure are not self-replicating RNA and do not include components necessary for viral replication.

It should also be understood that the immunogenic compositions, e.g, vaccines, of the present disclosure are not naturally-occurring. That is, the immunogenic compositions, e.g, vaccines, provided herein do not occur in nature.

### Exemplary Respiratory Syncytial Virus (RSV) Antigens

Antigens are proteins capable of inducing an immune response (e.g., causing an immune system to produce antibodies against the antigens). Herein, use of the term antigen encompasses immunogenic proteins and immunogenic fragments (an immunogenic fragment that induces (or is capable of inducing) an immune response to RSV), unless otherwise stated. It should be understood that the term “protein’ encompasses peptides and the term “antigen” encompasses antigenic fragments.

A number of different antigens are associated with RSV. RSV vaccines, as provided herein, comprise at least one (one or more) ribonucleic acid (RNA, e.g., mRNA) comprising an open reading frame encoding at least one RSV antigen. Non-limiting examples of RSV antigens are provided below.

Exemplary RSV antigens are provided in the Sequence Listing elsewhere herein. For example, the antigens may be encoded by (thus the RNA may comprise or consist of) any one of sequences set forth in SEQ ID NOS: 3, 7, 10, 13, 16, 19, 22, 24, 26, 28, 30, 32, 34, 37, 40, 43, 46, 49, 52, 54, 56, 58, 60, 63, 66, 69, 72, or 75. In some embodiments, the aforementioned sequences may further comprise a 5′ cap (e.g., 7mG(5′)ppp(5′)NlmpNp), a polyA tail, or a 5′ cap and a polyA tail).

It should be understood that the RSV vaccines of the present disclosure may comprise any of the RNA open reading frames (ORFs), or encode any of the protein ORFs, described herein, with or without a signal sequence. It should also be understood that the RSV vaccines of the present disclosure may include any 5′ untranslated region (UTR) and/or any 3′ UTR. Exemplary UTR sequences are provided in the Sequence Listing (e.g., SEQ ID NOS: 2, 4, 77, and 78); however, other UTR sequences (e.g., of the prior art) may be used or exchanged for any of the UTR sequences described herein. UTRs may also be omitted from the vaccine constructs provided herein.

At least two antigenic subgroups (A and B) of RSV are known to exist. This antigenic dimorphism is due primarily to difference in the surface G glycoproteins. Two surface glycoproteins, G and F, are present in the envelope and mediate attachment and fusion with cells of the respiratory epithelium. The F proteins also mediate coalescence of neighboring cells to form the characteristic syncytial cells for which the virus receives its name. The epidemiologic and biologic significance of the two antigenic variants of RSV is uncertain. Nonetheless, there is some evidence to suggest that Group A infections tend to be more severe.

The RSV genome is ˜15,000 nucleotides in length and is composed of a single strand of RNA with negative polarity. It has 10 genes encoding 11 proteins—there are 2 open reading frames of M2. The genome is transcribed sequentially from NS1 to L with reduction in expression levels along its length.

SH protein, G protein and F protein form the viral coat. The G protein is a surface protein that is heavily glycosylated and functions as the attachment protein. The F protein is another important surface protein that mediates fusion, allowing entry of the virus into the cell cytoplasm and also allowing the formation of syncytia. The F protein is homologous in both subtypes of RSV; antibodies directed at the F protein are neutralizing. In contrast, the G protein differs considerably between the two RSV subtypes. In some embodiments, a RSV vaccine comprises a RNA (e.g., mRNA) polynucleotide comprising an ORF encoding RSV F protein and a RNA (e.g., mRNA) polynucleotide comprising an ORF encoding RSV SH protein. In some embodiments, a RSV vaccine comprises a RNA (e.g., mRNA) polynucleotide comprising an ORF encoding RSV F protein and a RNA (e.g., mRNA) polynucleotide comprising an ORF encoding RSV G protein.

NS1 and NS2 inhibit type I interferon activity. In some embodiments, a RSV vaccine comprises a RNA (e.g., mRNA) polynucleotide comprising an ORF encoding RSV F protein and a RNA (e.g., mRNA) polynucleotide comprising an ORF encoding RSV NS1 and/or RSV NS2.

N encodes nucleocapsid protein that associates with the genomic RNA forming the nucleocapsid. In some embodiments, a RSV vaccine comprises a RNA (e.g., mRNA) polynucleotide comprising an ORF encoding RSV F protein and a RNA (e.g., mRNA) polynucleotide comprising an ORF encoding RSV nucleocapsid protein.

M encodes the Matrix protein required for viral assembly. In some embodiments, a RSV vaccine comprises a RNA (e.g., mRNA) polynucleotide comprising an ORF encoding RSV F protein and a RNA (e.g., mRNA) polynucleotide comprising an ORF encoding RSV Matrix protein.

Nucleolin at the cell surface is the receptor for the RSV fusion protein. Interference with the nucleolin-RSV fusion protein interaction has been shown to be therapeutic against RSV infection in cell cultures and animal models. In some embodiments, a RSV vaccine comprises a RNA (e.g., mRNA) polynucleotide comprising an ORF encoding RSV F protein and a RNA (e.g., mRNA) polynucleotide comprising an ORF encoding RSVnucleolin.

M2 is the second matrix protein also required for transcription and encodes M2-1 (elongation factor) and M2-2 (transcription regulation). M2 contains CD8 epitopes. In some embodiments, a RSV vaccine comprises a RNA (e.g., mRNA) polynucleotide comprising an ORF encoding RSV F protein and a RNA (e.g., mRNA) polynucleotide comprising an ORF encoding RSV second matrix protein.

L encodes the RNA polymerase. In some embodiments, a RSV vaccine comprises a RNA (e.g., mRNA) polynucleotide comprising an ORF encoding RSV F protein and a RNA (e.g., mRNA) polynucleotide comprising an ORF encoding RSV RNA polymerase (L).

The phosphoprotein P is a cofactor for the L protein. In some embodiments, a RSV vaccine comprises a RNA (e.g., mRNA) polynucleotide comprising an ORF encoding RSV F protein and a RNA (e.g., mRNA) polynucleotide comprising an ORF encoding RSV phosphoprotein P.

Some embodiments of the present disclosure provide RSV vaccines that include at least one RNA (e.g., mRNA) polynucleotide comprising an open reading frame encoding RSV glycoprotein F.

Some embodiments of the present invention disclose RSV vaccines that include at least one RNA (e.g. mRNA) polynucleotide comprising an open reading frame encoding a polypeptide in the post-fusion form. Some embodiments of the present invention disclose RSV vaccines that include at least one RNA (e.g. mRNA) polynucleotide comprising an open reading frame encoding a polypeptide in the pre-fusion form. In some embodiments, the polypeptides comprise glycoproteins in a prefusion conformation, for example, but not limited to, prefusion glycoprotein F or DS-CAV1. Without wishing to be bound by theory, certain polypeptides, when in a prefusion conformation, may contain more epitopes for neutralizing antibodies relative to the postfusion conformation of the same proteins. For example, prefusion glycoprotein F has a unique antigen site (“antigenic site 0”) at its membrane distal apex. Antigenic site 0 may, but not necessarily, comprise residues 62-69 and 196-209 of a RSV F protein sequence. In some instances, such as, but not limited to, prefusion glycoprotein F, prefusion polypeptides may exhibit many fold greater immune responses than those achieved with post-fusion polypeptides. Prefusion RSV glycoproteins and their methods of use are described in WO 2014/160463 and in WO 2017/172890, each of which is incorporated by reference herein in its entirety.

In some embodiments, RSV vaccines include at least one RNA (e.g., mRNA) polynucleotide comprising an open reading frame encoding glycoprotein F obtained from RSV strain A2 (RSV A2). Other RSV strains are encompassed by the present disclosure, including subtype A strains and subtype B strains.

### Nucleic Acids

The RSV vaccines of the present disclosure comprise at least one (one or more) ribonucleic acid (RNA) comprising an open reading frame encoding at least one RSV antigen. In some embodiments, the RNA is a messenger RNA (mRNA) comprising an open reading frame encoding at least one RSV antigen. In some embodiments, the RNA (e.g., mRNA) further comprises a (at least one) 5′ UTR, 3′ UTR, a polyA tail and/or a 5′ cap.

Nucleic acids comprise a polymer of nucleotides (nucleotide monomers), also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any ribonucleic acid that encodes a (at least one) protein (a naturally-occurring, non-naturally-occurring, or modified polymer of amino acids) and can be translated to produce the encoded protein in vitro, in vivo, in situ or ex vivo. The skilled artisan will appreciate that, except where otherwise noted, nucleic acid sequences set forth in the instant application may recite “T”s in a representative DNA sequence but where the sequence represents RNA (e.g., mRNA), the “T”s would be substituted for “U”s. Thus, any of the DNAs disclosed and identified by a particular sequence identification number herein also disclose the corresponding RNA (e.g., mRNA) sequence complementary to the DNA, where each “T” of the DNA sequence is substituted with “U.” Likewise, any of the RNAs disclosed and identified by a particular sequence identification number herein also disclose the corresponding DNA sequence complementary to the RNA, where each “U” of the RNA sequence is substituted with “T.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in a vaccine of the present disclosure.

### Variants

In some embodiments, an RNA of the present disclosure encodes an RSV antigen variant. Antigen or other polypeptide variants refers to molecules that differ in their amino acid sequence from a wild-type, native or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art and exemplary such assays are set forth in the Examples section. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, an RSV vaccine comprises an mRNA ORF comprising a nucleotide sequence identified by any one of the sequences provided herein (see e.g., Sequence Listing), or comprising a nucleotide sequence at least 85%, at least 90%, at least 95%, at least 96%, at least 97%, at least 98%, or at least 99% identical to a nucleotide sequence identified by any one of the sequence provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 85%, 86%, 87%, 88%, 89%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an RNA (e.g., mRNA) vaccine.

As recognized by those skilled in the art, protein fragments, functional protein domains, and homologous proteins are also considered to be within the scope of RSV antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to the RSV pathogen. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a vaccine includes at least one RNA polynucleotide comprising an open reading frame encoding at least one antigenic polypeptide comprising at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, RSV RNA vaccines may include one or more stabilizing elements. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, RSV RNA vaccines include a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, RSV RNA vaccines do not comprise a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

In some embodiments, RSV RNA vaccines may or may not contain an enhancer and/or promoter sequence, which may be modified or unmodified or which may be activated or inactivated. In some embodiments, the histone stem-loop is generally derived from histone genes, and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures, but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, RSV RNA vaccines may have one or more AU-rich sequences removed. These sequences, sometimes referred to as AURES are destabilizing sequences found in the 3′UTR. The AURES may be removed from the RNA vaccines. Alternatively the AURES may remain in the RNA vaccine.

### Signal Peptides

In some embodiments, an RSV vaccine comprises a RNA comprising an ORF that encodes a signal peptide fused to the RSV antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by a ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than RSV antigens in nature) are known in the art and can be tested for desired properties and then incorporated into a nucleic acid of the disclosure. In some embodiments, the signal peptide is a bovine prolactin signal peptide. For example, the bovine prolactin signal peptide may comprise sequence MDSKGSSQKGSRLLLLLVVSNLLLPQGVVG (SEQ ID NO: 79). Other signal peptide sequences may also be used. For example, the signal peptide may comprise one of the following sequences: MDWTWILFLVAAATRVHS (SEQ ID NO: 80); METPAQLLFLLLLWLPDTTG (SEQ ID NO:81); MLGSNSGQRVVFTILLLLVAPAYS (SEQ ID NO: 82); MKCLLYLAFLFIGVNCA (SEQ ID NO: 83); MWLVSLAIVTACAGA (SEQ ID NO: 84); or MELLILKANAITTILTAVTFCFASG (SEQ ID NO:100).

### Fusion Proteins

In some embodiments, an RSV RNA vaccine of the present disclosure includes an RNA encoding an antigenic fusion protein. Thus, the encoded antigen or antigens may include two or more proteins (e.g., protein and/or protein fragment) joined together. Alternatively, the protein to which a protein antigen is fused does not promote a strong immune response to itself, but rather to the RSV antigen. Antigenic fusion proteins, in some embodiments, retain the functional property from each original protein.

### Scaffold Moieties

The RNA (e.g., mRNA) vaccines as provided herein, in some embodiments, encode fusion proteins which comprise RSV antigens linked to scaffold moieties. In some embodiments, such scaffold moieties impart desired properties to an antigen encoded by a nucleic acid of the disclosure. For example scaffold proteins may improve the immunogenicity of an antigen, e.g., by altering the structure of the antigen, altering the uptake and processing of the antigen, and/or causing the antigen to bind to a binding partner.

In some embodiments, the scaffold moiety is protein that can self-assemble into protein nanoparticles that are highly symmetric, stable, and structurally organized, with diameters of 10-150 nm, a highly suitable size range for optimal interactions with various cells of the immune system. In some embodiments, viral proteins or virus-like particles can be used to form stable nanoparticle structures. Examples of such viral proteins are known in the art. For example, in some embodiments, the scaffold moiety is a hepatitis B surface antigen (HBsAg). HBsAg forms spherical particles with an average diameter of ˜22 nm and which lacked nucleic acid and hence are non-infectious (Lopez-Sagaseta, J. et al. Computational and Structural Biotechnology Journal 14 (2016) 58-68). In some embodiments, the scaffold moiety is a hepatitis B core antigen (HBcAg) self-assembles into particles of 24-31 nm diameter, which resembled the viral cores obtained from HBV-infected human liver. HBcAg produced in self-assembles into two classes of differently sized nanoparticles of 300 Å and 360 Å diameter, corresponding to 180 or 240 protomers. In some embodiments an RSV antigen is fused to HBsAG or HBcAG to facilitate self-assembly of nanoparticles displaying the RSV antigen.

In another embodiment, bacterial protein platforms may be used. Non-limiting examples of these self-assembling proteins include ferritin, lumazine and encapsulin.

Ferritin is a protein whose main function is intracellular iron storage. Ferritin is made of 24 subunits, each composed of a four-alpha-helix bundle, that self-assemble in a quaternary structure with octahedral symmetry (Cho K. J. et al. J Mol Biol. 2009; 390:83-98). Several high-resolution structures of ferritin have been determined, confirming that Helicobacter pylori ferritin is made of 24 identical protomers, whereas in animals, there are ferritin light and heavy chains that can assemble alone or combine with different ratios into particles of 24 subunits (Granier T. et al. J Biol Inorg Chem. 2003; 8:105-111; Lawson D. M. et al. Nature. 1991; 349:541-544). Ferritin self-assembles into nanoparticles with robust thermal and chemical stability. Thus, the ferritin nanoparticle is well-suited to carry and expose antigens.

Lumazine synthase (LS) is also well-suited as a nanoparticle platform for antigen display. LS, which is responsible for the penultimate catalytic step in the biosynthesis of riboflavin, is an enzyme present in a broad variety of organisms, including archaea, bacteria, fungi, plants, and eubacteria (Weber S. E. Flavins and Flavoproteins. Methods and Protocols, Series: Methods in Molecular Biology. 2014). The LS monomer is 150 amino acids long, and consists of beta-sheets along with tandem alpha-helices flanking its sides. A number of different quaternary structures have been reported for LS, illustrating its morphological versatility: from homopentamers up to symmetrical assemblies of 12 pentamers forming capsids of 150 Å diameter. Even LS cages of more than 100 subunits have been described (Zhang X. et al. J Mol Biol. 2006; 362:753-770).

Encapsulin, a novel protein cage nanoparticle isolated from thermophile Thermotoga maritima, may also be used as a platform to present antigens on the surface of self-assembling nanoparticles. Encapsulin is assembled from 60 copies of identical 31 kDa monomers having a thin and icosahedral T=1 symmetric cage structure with interior and exterior diameters of 20 and 24 nm, respectively (Sutter M. et al. Nat Struct Mol Biol. 2008, 15: 939-947). Although the exact function of encapsulin in T. maritima is not clearly understood yet, its crystal structure has been recently solved and its function was postulated as a cellular compartment that encapsulates proteins such as DyP (Dye decolorizing peroxidase) and Flp (Ferritin like protein), which are involved in oxidative stress responses (Rahmanpour R. et al. FEBS J. 2013, 280: 2097-2104).

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding an RSV antigen). In some embodiments, a codon optimized sequence shares less than 90% sequence identity to a naturally-occurring or wild-type sequence (e.g., a naturally-occurring or wild-type mRNA sequence encoding an RSV antigen). In some embodiments, a codon optimized sequence shares less than 85% sequence identity to a naturally-occurring or wild-type sequence (e.g., a naturally-occurring or wild-type mRNA sequence encoding an RSV antigen). In some embodiments, a codon optimized sequence shares less than 80% sequence identity to a naturally-occurring or wild-type sequence (e.g., a naturally-occurring or wild-type mRNA sequence encoding an RSV antigen). In some embodiments, a codon optimized sequence shares less than 75% sequence identity to a naturally-occurring or wild-type sequence (e.g., a naturally-occurring or wild-type mRNA sequence encoding an RSV antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding an RSV antigen). In some embodiments, a codon optimized sequence shares between 65% and 75% or about 80% sequence identity to a naturally-occurring or wild-type sequence (e.g., a naturally-occurring or wild-type mRNA sequence encoding an RSV antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than an RSV antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, at least one RNA (e.g., mRNA) of an RSV vaccines of the present disclosure is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

RSV RNA vaccines of the present disclosure comprise, in some embodiments, at least one nucleic acid (e.g., RNA) comprising an open reading frame encoding at least one RSV antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids comprising at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a RNA nucleic acid of the disclosure comprises 1-methyl-pseudouridine (m1ψ) substitutions at one or more or all uridine positions of the nucleic acid.

In some embodiments, a RNA nucleic acid of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises pseudouridine (W) substitutions at one or more or all uridine positions of the nucleic acid.

In some embodiments, a RNA nucleic acid of the disclosure comprises pseudouridine (ψ) substitutions at one or more or all uridine positions of the nucleic acid and 5-methyl cytidine substitutions at one or more or all cytidine positions of the nucleic acid.

In some embodiments, a RNA nucleic acid of the disclosure comprises uridine at one or more or all uridine positions of the nucleic acid.

In some embodiments, nucleic acids (e.g., RNA nucleic acids, such as mRNA nucleic acids) are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the polyA tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The nucleic acids may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

### Untranslated Regions (UTRs)

The nucleic acids of the present disclosure may comprise one or more regions or parts which act or function as an untranslated region. Where nucleic acids are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 85), where R is a purine (adenine or guanine) three bases upstream of the start codon (AUG), which is followed by another ‘G’0.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived a-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 86) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-0) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a sequence selected from SEQ ID NO: 2 and SEQ ID NO: 77.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) (SEQ ID NO: 87) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

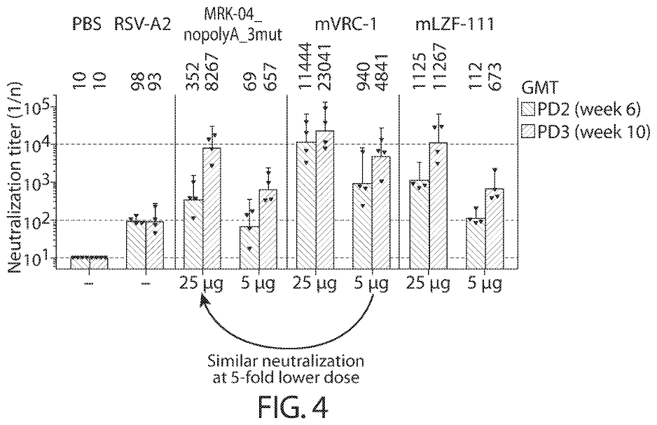

3′ UTRs may be heterologous or synthetic. With respect to 3′ UTRs, globin UTRs, including Xenopus β-globin UTRs and human β-globin UTRs are known in the art (U.S. Pat. Nos. 8,278,063, 9,012,219, US20110086907). A modified β-globin construct with enhanced stability in some cell types by cloning two sequential human β-globin 3′UTRs head to tail has been developed and is well known in the art (US2012/0195936, WO2014/071963). In addition a2-globin, a1-globin, UTRs and mutants thereof are also known in the art (WO2015101415, WO2015024667). Other 3′ UTRs described in the mRNA constructs in the non-patent literature include CYBA (Ferizi et al., 2015) and albumin (Thess et al., 2015). Other exemplary 3′ UTRs include that of bovine or human growth hormone (wild type or modified) (WO2013/185069, US20140206753, WO2014/152774), rabbit β globin and hepatitis B virus (HBV), α-globin 3′ UTR and Viral VEEV 3′ UTR sequences are also known in the art. In some embodiments, the sequence UUUGAAUU (WO2014/144196) is used. In some embodiments, 3′ UTRs of human and mouse ribosomal protein are used. Other examples include rps9 3′UTR (WO2015101414), FIG. 4 (WO2015/101415), and human albumin 7 (WO2015/101415).

In some embodiments, a 3′ UTR of the present disclosure comprises a sequence selected from SEQ ID NO: 4 and SEQ ID NO: 78.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3″ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### In Vitro Transcription of RNA

cDNA encoding the polynucleotides described herein may be transcribed using an in vitro transcription (IVT) system. In vitro transcription of RNA is known in the art and is described in International Publication WO/2014/152027, which is incorporated by reference herein in its entirety.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of a RNA polynucleotide, for example, but not limited to RSV RNA, e.g. RSV mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes a RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a polyA tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a polyA tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

### Solid-Phase Chemical Synthesis.

Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

### Liquid Phase Chemical Synthesis.

The synthesis of nucleic acids of the present disclosure by the sequential addition of monomer building blocks may be carried out in a liquid phase.

### Combination of Synthetic Methods.

The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present invention may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Pharmaceutical Formulations

Provided herein are compositions (e.g., pharmaceutical compositions), methods, kits and reagents for prevention or treatment of RSV in humans and other mammals, for example. RSV RNA (e.g., mRNA) vaccines can be used as therapeutic or prophylactic agents. They may be used in medicine to prevent and/or treat infectious disease.

In some embodiments, an RSV vaccine containing RNA polynucleotides as described herein can be administered to a subject (e.g., a mammalian subject, such as a human subject), and the RNA polynucleotides are translated in vivo to produce an antigenic polypeptide (antigen).

An “effective amount” of an RSV vaccine is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of an RSV vaccine provides an induced or boosted immune response as a function of antigen production in the cells of the subject. In some embodiments, an effective amount of the RSV RNA vaccine containing RNA polynucleotides comprising at least one chemical modifications are more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form.

Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, RNA vaccines (including polynucleotides and their encoded polypeptides) in accordance with the present disclosure may be used for treatment or prevention of RSV. RSV RNA vaccines may be administered prophylactically or therapeutically as part of an active immunization scheme to healthy individuals or early in infection during the incubation phase or during active infection after onset of symptoms. In some embodiments, the amount of RNA vaccines of the present disclosure provided to a cell, a tissue or a subject may be an amount effective for immune prophylaxis.

RSV RNA (e.g., mRNA) vaccines may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years. In exemplary embodiments, the time of administration between the initial administration of the prophylactic composition and the booster may be, but is not limited to, 1 week, 2 weeks, 3 weeks, 1 month, 2 months, 3 months, 6 months or 1 year.

In some embodiments, RSV RNA vaccines may be administered intramuscularly, intranasally or intradermally, similarly to the administration of inactivated vaccines known in the art.

The RSV RNA vaccines may be utilized in various settings depending on the prevalence of the infection or the degree or level of unmet medical need. As a non-limiting example, the RNA vaccines may be utilized to treat and/or prevent a variety of infectious disease. RNA vaccines have superior properties in that they produce much larger antibody titers, better neutralizing immunity, produce more durable immune responses, and/or produce responses earlier than commercially available vaccines.

Provided herein are pharmaceutical compositions including RSV RNA vaccines and RNA vaccine compositions and/or complexes optionally in combination with one or more pharmaceutically acceptable excipients.

RSV RNA (e.g., mRNA) vaccines may be formulated or administered alone or in conjunction with one or more other components. For instance, RSV RNA vaccines (vaccine compositions) may comprise other components including, but not limited to, adjuvants.

In some embodiments, RSV RNA vaccines do not include an adjuvant (they are adjuvant free).

RSV RNA (e.g., mRNA) vaccines may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, RSV RNA vaccines are administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, RSV RNA vaccines are formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with RSV RNA vaccines (e.g., for transplantation into a subject), hyaluronidase, nanoparticle mimics and combinations thereof.

### Lipid Nanoparticles (LNPs)

In some embodiments, RSV RNA (e.g., mRNA) vaccines of the disclosure are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

In some embodiments, an ionizable cationic lipid of the disclosure comprises a compound of Formula (I):

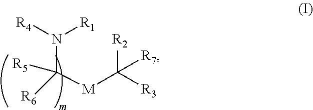

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl comprising one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle comprising one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl comprising one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

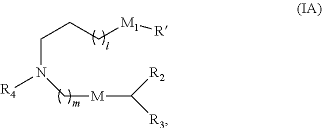

or a salt or isomer thereof, wherein 1 is selected from 1, 2, 3, 4, and 5; m is selected from 5, 6, 7, 8, and 9; M1 is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

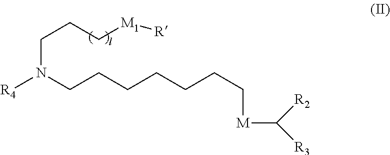

or a salt or isomer thereof, wherein 1 is selected from 1, 2, 3, 4, and 5; M1 is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

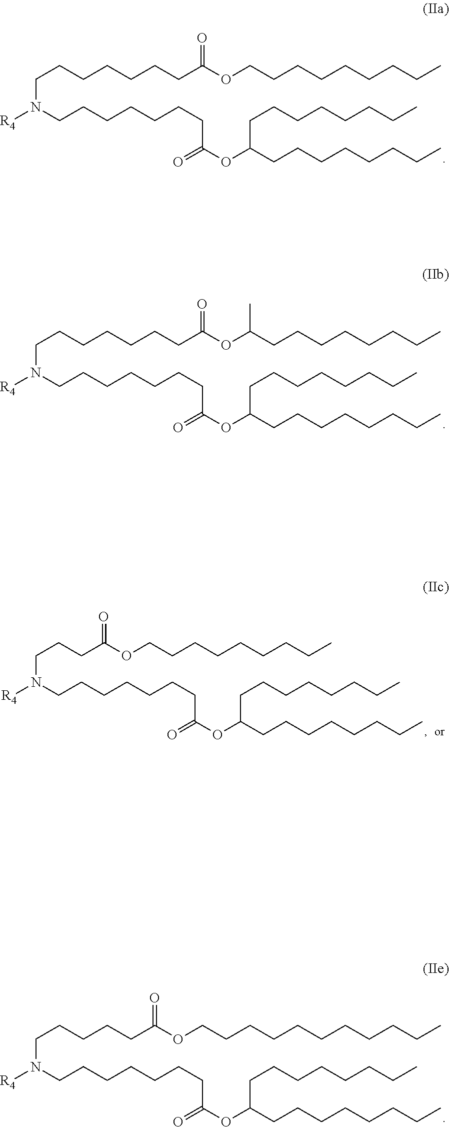

or a salt or isomer thereof, wherein R₄ is as described herein.

In some embodiments, a subset of compounds of Formula (I) includes those of Formula (IId):

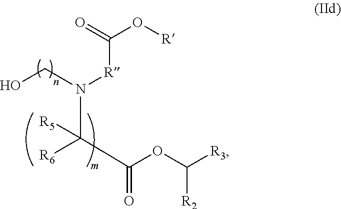

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable cationic lipid of the disclosure comprises a compound having structure:

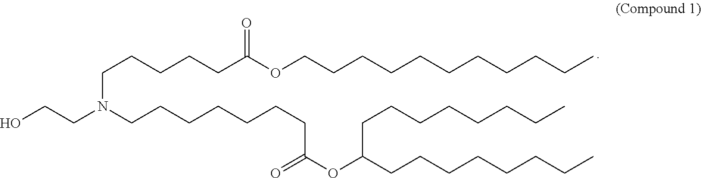 [SMILES: CCCCCCCCCCCOC(=O)CCCCCN(CCO)CCCCCCCC(=O)OC(CCCCCCCC)CCCCCCCC]

In some embodiments, an ionizable cationic lipid of the disclosure comprises a compound having structure:

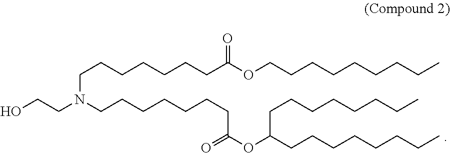 [SMILES: CCCCCCCCCOC(=O)CCCCCCCN(CCO)CCCCCCCC(=O)OC(CCCCCCCC)CCCCCCCC]

In some embodiments, an ionizable cationic lipid is selected from the group consisting of 2,2-dilinoleyl-4-dimethylaminoethyl-[1,3]-dioxolane (DLin-KC2-DMA), dilinoleyl-methyl-4-dimethylaminobutyrate (DLin-MC3-DMA), di((Z)-non-2-en-1-yl) 9-((4-(dimethylamino)butanoyl)oxy)heptadecanedioate (L319), (13Z,16Z)—N,N-dimethyl-3-nonyldocosa-13,16-dien-1-amine, (12Z,15Z)—N,N-dimethyl-2-nonylhenicosa-12,15-dien-1-amine, and N,N-dimethyl-1-[(1S,2R)-2-octylcyclopropyl]heptadecan-8-amine. In one embodiment, the lipid is (13Z,16Z)—N,N-dimethyl-3-nonyldocosa-13,16-dien-1-amine or N,N-dimethyl-1-[(1S,2R)-2-octylcyclopropyl]heptadecan-8-amine, each of which are described in PCT/US2011/052328, the entire contents of which are hereby incorporated by reference. In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable cationic lipid of Compound 1, wherein the non-cationic lipid is DSPC, the structural lipid that is cholesterol, and the PEG lipid is PEG-DMG.

In some embodiments, a LNP of the disclosure comprises an ionizable cationic lipid selected from the group consisting of (2S)-1-({6-[(3β))-cholest-5-en-3-yloxy]hexyl}oxy)-N,N-dimethyl-3-[(9 Z)-octadec-9-en-1-yloxy]propan-2-amine; (13Z,16Z)—N,N-dimethyl-3-nonyldocosa-13,16-dien-1-amine; and N,N-dimethyl-1-[(1S,2R)-2-octylcyclopropyl]heptadecan-8-amine; or a pharmaceutically acceptable salt thereof, or a stereoisomer of any of the foregoing.

In some embodiments, the LNP comprises 30-75 mole % ionizable cationic lipid and 0.1-20 mole % PEG-lipid.

In some embodiments, the LNP further comprises one or more non-cationic lipids selected from a phospholipid, a phospholipid derivative, a fatty acid, a sterol, or a combination thereof. In some embodiments, the sterol is cholesterol, stigmasterol or stigmastanol.

In some embodiments, the phospholipid is selected from phosphatidylserine, 1,2-Distearoyl-sn-glycero-3-phosphocholine (DSPC), 1,2-dipalmitoleoyl-sn-glycero-3-phosphocholine, 1,2-dimyristoyl-sn-glycero-3-phosphocholine (DMPC), dilauroylphosphatidylcholine (DLPC), 1,2-dieicosenoyl-sn-glycero-3-phosphocholine, and 1,2-dioleoyl-sn-glycero-3-phosphocholine (DOPC).

In some embodiments, the PEG-lipid is 1,2-Dimyristoyl-sn-glycerol methoxypolyethylene glycol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG-lipid is PEG coupled to dimyristoylglycerol (PEG-DMG), e.g., as described in Abrams et al., 2010, Molecular Therapy 18(1):171, and U.S. Patent Application Publication Nos. US 2006/0240554 and US 2008/0020058, including for example, 2KPEG/PEG200-DMG.

In some embodiments, the LNP comprises 20-99.8 mole % ionizable cationic lipids, 0.1-65 mole % non-cationic lipids, and 0.1-20 mole % PEG-lipid.

In some embodiments, the non-cationic lipids comprise a mixture of cholesterol and DSPC.

In some embodiments, the LNP comprises 34-59 mole % ionizable cationic lipids selected from the group consisting of (2S)-1-({6-[(3))-cholest-5-en-3-yloxy]hexyl}oxy)-N,N-dimethyl-3-[(9 Z)-octadec-9-en-1-yloxy]propan-2-amine; (13Z,16Z)—N,N-dimethyl-3-nonyldocosa-13,16-dien-1-amine; and N,N-dimethyl-1-[(1S,2R)-2-octylcyclopropyl]heptadecan-8-amine, 30-48 mole % cholesterol, 10-24% DSPC and 1-2 mole % PEG-DMG.

In some embodiments, the LNP comprises 58 mole % cationic lipid, 30 mole % cholesterol, 10 mole % DSPC, and 2 mole % PEG-2000 DMG.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Multivalent Vaccines

The RSV vaccines, as provided herein, may include an RNA (e.g. mRNA) or multiple RNAs encoding two or more antigens of the same or different RSV species. In some embodiments, an RSV vaccine includes an RNA or multiple RNAs encoding two or more antigens selected from glycoprotein G (G), glycoprotein F (F), matrix protein (M), small hydrophobic protein (SH), nonstructural protein 1 (NS1), and nonstructural protein 2 (NS2). In some embodiments, the RNA (at least one RNA) of an RSV vaccine may encode 1, 2, 3, 4, 5, 6, 7, 8, 9, 10, 11, 12, or more antigens.

In some embodiments, an RSV vaccine includes an RNA or multiple RNAs encoding two or more antigens selected from glycoprotein G and glycoprotein F. In some embodiments, an RSV vaccine includes an RNA or multiple RNAs encoding two or more antigens selected from glycoprotein G and matrix protein. In some embodiments, an RSV vaccine includes an RNA or multiple RNAs encoding two or more antigens selected from glycoprotein F and matrix protein.

In some embodiments, two or more different RNA (e.g., mRNA) encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Combination Vaccines

The RSV vaccines, as provided herein, may include an RNA or multiple RNAs encoding two or more antigens of the same or different RSV strains. Also provided herein are combination vaccines that include RNA encoding one or more RSV antigen(s) and one or more antigen(s) of a different organisms (e.g., bacterial and/or viral organism). Thus, the vaccines of the present disclosure may be combination vaccines that target one or more antigens of the same strain/species, or one or more antigens of different strains/species, e.g., antigens which induce immunity to organisms which are found in the same geographic areas where the risk of RSV infection is high or organisms to which an individual is likely to be exposed to when exposed to RSV.

### Dosing/Administration

Provided herein are compositions (e.g., pharmaceutical compositions), methods, kits and reagents for prevention and/or treatment of RSV in humans and other mammals. RSV RNA vaccines can be used as therapeutic or prophylactic agents. In some aspects, the RNA vaccines of the disclosure are used to provide prophylactic protection from RSV. In some aspects, the RNA vaccines of the disclosure are used to treat an RSV infection. In some embodiments, the RSV vaccines of the present disclosure are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, the RSV vaccines are administered to a subject (e.g., a mammalian subject, such as a human subject) in an effective amount to induce an antigen-specific immune response. The RNA encoding the RSV antigen is expressed and translated in vivo to produce the antigen, which then stimulates an immune response in the subject.

Prophylactic protection from RSV can be achieved following administration of an RSV RNA vaccine of the present disclosure. Vaccines can be administered once, twice, three times, four times or more but it is likely sufficient to administer the vaccine once (optionally followed by a single booster). It is possible, although less desirable, to administer the vaccine to an infected individual to achieve a therapeutic response. Dosing may need to be adjusted accordingly.

A method of eliciting an immune response in a subject against RSV is provided in aspects of the present disclosure. The method involves administering to the subject an RSV RNA vaccine comprising at least one RNA (e.g., mRNA) comprising an open reading frame encoding at least one RSV antigen, thereby inducing in the subject an immune response specific to RSV antigen, wherein anti-antigen antibody titer in the subject is increased following vaccination relative to anti-antigen antibody titer in a subject vaccinated with a prophylactically effective dose of a traditional vaccine against the RSV. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against the RSV or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against the RSV or an unvaccinated subject.

A method of eliciting an immune response in a subject against an RSV is provided in other aspects of the disclosure. The method involves administering to the subject an RSV RNA vaccine comprising at least one RNA polynucleotide comprising an open reading frame encoding at least one RSV antigen, thereby inducing in the subject an immune response specific to RSV antigen, wherein the immune response in the subject is equivalent to an immune response in a subject vaccinated with a traditional vaccine against the RSV at 2 times to 100 times the dosage level relative to the RNA vaccine.

In some embodiments, the immune response in the subject is equivalent to an immune response in a subject vaccinated with a traditional vaccine at twice the dosage level relative to the RSV RNA vaccine. In some embodiments, the immune response in the subject is equivalent to an immune response in a subject vaccinated with a traditional vaccine at three times the dosage level relative to the RSV RNA vaccine. In some embodiments, the immune response in the subject is equivalent to an immune response in a subject vaccinated with a traditional vaccine at 4 times, 5 times, 10 times, 50 times, or 100 times the dosage level relative to the RSV RNA vaccine. In some embodiments, the immune response in the subject is equivalent to an immune response in a subject vaccinated with a traditional vaccine at 10 times to 1000 times the dosage level relative to the RSV RNA vaccine. In some embodiments, the immune response in the subject is equivalent to an immune response in a subject vaccinated with a traditional vaccine at 100 times to 1000 times the dosage level relative to the RSV RNA vaccine.

In other embodiments, the immune response is assessed by determining antibody titer in the subject. In other embodiments, the ability of serum or antibody from an immunized subject is tested for its ability to neutralize viral uptake or reduce RSV transformation of human B lymphocytes. In other embodiments, the ability to promote a robust T cell response(s) is measured using art recognized techniques.

Other aspects the disclosure provide methods of eliciting an immune response in a subject against an RSV by administering to the subject an RSV RNA vaccine comprising at least one RNA polynucleotide comprising an open reading frame encoding at least one RSV antigen, thereby inducing in the subject an immune response specific to RSV antigen, wherein the immune response in the subject is induced 2 days to 10 weeks earlier relative to an immune response induced in a subject vaccinated with a prophylactically effective dose of a traditional vaccine against the RSV. In some embodiments, the immune response in the subject is induced in a subject vaccinated with a prophylactically effective dose of a traditional vaccine at 2 times to 100 times the dosage level relative to the RNA vaccine.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against an RSV by administering to the subject an RSV RNA vaccine comprising an open reading frame encoding a first antigen, wherein the RNA polynucleotide does not include a stabilization element, and wherein an adjuvant is not co-formulated or co-administered with the vaccine.

RSV RNA (e.g., mRNA) vaccines may be administered by any route which results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. RSV RNA (e.g., mRNA) vaccines compositions are typically formulated in dosage unit form for ease of administration and uniformity of dosage. It will be understood, however, that the total daily usage of RSV RNA (e.g., mRNA) vaccines compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of an RSV vaccine, as provided herein, may be as low as 20 μg, administered for example as a single dose or as two 10 μg doses. In some embodiments, the effective amount is a total dose of 20 μg-200 μg. For example, the effective amount may be a total dose of 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg or 200 μg. In some embodiments, the effective amount is a total dose of 25 μg-200 μg. In some embodiments, the effective amount is a total dose of 50 μg-200 μg.

In some embodiments, RSV RNA (e.g., mRNA) vaccines compositions may be administered at dosage levels sufficient to deliver 0.0001 mg/kg to 100 mg/kg, 0.001 mg/kg to 0.05 mg/kg, 0.005 mg/kg to 0.05 mg/kg, 0.001 mg/kg to 0.005 mg/kg, 0.05 mg/kg to 0.5 mg/kg, 0.01 mg/kg to 50 mg/kg, 0.1 mg/kg to 40 mg/kg, 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No. WO2013/078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, RSV RNA (e.g., mRNA) vaccines compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, RSV RNA (e.g., mRNA) vaccine compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In some embodiments, RSV RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.0100 mg, 0.025 mg, 0.050 mg, 0.075 mg, 0.100 mg, 0.125 mg, 0.150 mg, 0.175 mg, 0.200 mg, 0.225 mg, 0.250 mg, 0.275 mg, 0.300 mg, 0.325 mg, 0.350 mg, 0.375 mg, 0.400 mg, 0.425 mg, 0.450 mg, 0.475 mg, 0.500 mg, 0.525 mg, 0.550 mg, 0.575 mg, 0.600 mg, 0.625 mg, 0.650 mg, 0.675 mg, 0.700 mg, 0.725 mg, 0.750 mg, 0.775 mg, 0.800 mg, 0.825 mg, 0.850 mg, 0.875 mg, 0.900 mg, 0.925 mg, 0.950 mg, 0.975 mg, or 1.0 mg. Higher and lower dosages and frequency of administration are encompassed by the present disclosure. For example, an RSV RNA (e.g., mRNA) vaccine composition may be administered three or four times.

In some embodiments, RSV RNA (e.g., mRNA) vaccine compositions may be administered twice (e.g., Day 0 and Day 7, Day 0 and Day 14, Day 0 and Day 21, Day 0 and Day 28, Day 0 and Day 60, Day 0 and Day 90, Day 0 and Day 120, Day 0 and Day 150, Day 0 and Day 180, Day 0 and 3 months later, Day 0 and 6 months later, Day 0 and 9 months later, Day 0 and 12 months later, Day 0 and 18 months later, Day 0 and 2 years later, Day 0 and 5 years later, or Day 0 and 10 years later) at a total dose of or at dosage levels sufficient to deliver a total dose of 0.010 mg, 0.025 mg, 0.100 mg or 0.400 mg.

In some embodiments, the RSV RNA (e.g., mRNA) vaccine for use in a method of vaccinating a subject is administered the subject a single dosage of between 10 μg/kg and 400 μg/kg of the nucleic acid vaccine in an effective amount to vaccinate the subject. In some embodiments, the RNA vaccine for use in a method of vaccinating a subject is administered the subject a single dosage of between 10 μg and 400 μg of the nucleic acid vaccine in an effective amount to vaccinate the subject. In some embodiments, an RSV RNA (e.g., mRNA) vaccine for use in a method of vaccinating a subject is administered to the subject as a single dosage of 25-1000 μg (e.g., a single dosage of mRNA encoding an RSV antigen). In some embodiments, an RSV RNA vaccine is administered to the subject as a single dosage of 25, 50, 100, 150, 200, 250, 300, 350, 400, 450, 500, 550, 600, 650, 700, 750, 800, 850, 900, 950 or 1000 μg. For example, an RSV RNA vaccine may be administered to a subject as a single dose of 25-100, 25-500, 50-100, 50-500, 50-1000, 100-500, 100-1000, 250-500, 250-1000, or 500-1000 μg. In some embodiments, an RSV RNA (e.g., mRNA) vaccine for use in a method of vaccinating a subject is administered to the subject as two dosages, the combination of which equals 25-1000 μg of the RSV RNA (e.g., mRNA) vaccine.

An RSV RNA (e.g., mRNA) vaccine pharmaceutical composition described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the RSV RNA (e.g., mRNA) vaccine, wherein the RSV RNA vaccine is formulated in an effective amount to produce an antigen specific immune response in a subject (e.g., production of antibodies specific to an anti-RSV antigen). “An effective amount” is a dose of an RSV RNA (e.g., mRNA) vaccine effective to produce an antigen-specific immune response. Also provided herein are methods of inducing an antigen-specific immune response in a subject.

As used herein, an immune response to a composition (e.g., mRNA formulated with or without LNP) of the present disclosure is the development in a subject of a humoral and/or a cellular immune response to a (one or more) RSV protein(s) present in the composition. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function, and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells.

In some embodiments, the antigen-specific immune response is characterized by measuring an anti-RSV antigen antibody titer produced in a subject administered an RSV RNA (e.g., mRNA) vaccine as provided herein. An antibody titer is a measurement of the amount of antibodies within a subject, for example, antibodies that are specific to a particular antigen (e.g., an anti-RSV antigen) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the RSV RNA (e.g., mRNA) vaccine.

In some embodiments, an anti-RSV antigen antibody titer produced in a subject is increased by at least 1 log relative to a control. For example, anti-RSV antigen antibody titer produced in a subject may be increased by at least 1.5, at least 2, at least 2.5, or at least 3 log relative to a control. In some embodiments, the anti-RSV antigen antibody titer produced in the subject is increased by 1, 1.5, 2, 2.5 or 3 log relative to a control. In some embodiments, the anti-RSV antigen antibody titer produced in the subject is increased by 1-3 log relative to a control. For example, the anti-RSV antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-RSV antigen antibody titer produced in a subject is increased at least 2 times relative to a control. For example, the anti-RSV antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-RSV antigen antibody titer produced in the subject is increased 2, 3, 4, 5, 6, 7, 8, 9, or 10 times relative to a control. In some embodiments, the anti-RSV antigen antibody titer produced in a subject is increased 2-10 times relative to a control. For example, the anti-RSV antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

A control, in some embodiments, is the anti-RSV antigen antibody titer produced in a subject who has not been administered an RSV RNA (e.g., mRNA) vaccine. In some embodiments, a control is an anti-RSV antigen antibody titer produced in a subject administered a recombinant or purified RSV protein vaccine. Recombinant protein vaccines typically include protein antigens that either have been produced in a heterologous expression system (e.g., bacteria or yeast) or purified from large amounts of the pathogenic organism.

In some embodiments, the ability of an RSV vaccine to be effective is measured in a murine model. For example, the RSV vaccines may be administered to a murine model and the murine model assayed for induction of neutralizing antibody titers. Viral challenge studies may also be used to assess the efficacy of a vaccine of the present disclosure. For example, the RSV vaccines may be administered to a murine model, the murine model challenged with RSV, and the murine model assayed for survival and/or immune response (e.g., neutralizing antibody response).

In some embodiments, the ability of an RSV vaccine to be effective is measured in a non-human primate (e.g., African green monkey) model. For example, the RSV vaccines may be administered to a non-human primate model and the murine model assayed for induction of neutralizing antibody titers. Viral challenge studies may also be used to assess the efficacy of a vaccine of the present disclosure. For example, the RSV vaccines may be administered to a non-human primate model, the non-human primate model challenged with RSV, and the non-human primate model assayed for survival and/or immune response (e.g., neutralizing antibody response).

In some embodiments, an effective amount of an RSV RNA (e.g., mRNA) vaccine is a dose that is reduced compared to the standard of care dose of a recombinant RSV protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified RSV protein vaccine, or a live attenuated or inactivated RSV vaccine, or an RSV VLP vaccine, that a physician/clinician or other medical professional would administer to a subject to treat or prevent RSV, or an RSV-related condition, while following the standard of care guideline for treating or preventing RSV, or an RSV-related condition.

In some embodiments, the anti-RSV antigen antibody titer produced in a subject administered an effective amount of an RSV RNA vaccine is equivalent to an anti-RSV antigen antibody titer produced in a control subject administered a standard of care dose of a recombinant or purified RSV protein vaccine, or a live attenuated or inactivated RSV vaccine, or an RSV VLP vaccine.

In some embodiments, an effective amount of an RSV RNA (e.g., mRNA) vaccine is a dose equivalent to an at least 2-fold reduction in a standard of care dose of a recombinant or purified RSV protein vaccine. For example, an effective amount of an RSV RNA vaccine may be a dose equivalent to an at least 3-fold, at least 4-fold, at least 5-fold, at least 6-fold, at least 7-fold, at least 8-fold, at least 9-fold, or at least 10-fold reduction in a standard of care dose of a recombinant or purified RSV protein vaccine. In some embodiments, an effective amount of an RSV RNA vaccine is a dose equivalent to an at least 100-fold, at least 500-fold, or at least 1000-fold reduction in a standard of care dose of a recombinant or purified RSV protein vaccine. In some embodiments, an effective amount of an RSV RNA vaccine is a dose equivalent to a 2-, 3-, 4-, 5-, 6-, 7-, 8-, 9-, 10-, 20-, 50-, 100-, 250-, 500-, or 1000-fold reduction in a standard of care dose of a recombinant or purified RSV protein vaccine. In some embodiments, the anti-RSV antigen antibody titer produced in a subject administered an effective amount of an RSV RNA vaccine is equivalent to an anti-RSV antigen antibody titer produced in a control subject administered the standard of care dose of a recombinant or protein RSV protein vaccine, or a live attenuated or inactivated RSV vaccine, or an RSV VLP vaccine. In some embodiments, an effective amount of an RSV RNA (e.g., mRNA) vaccine is a dose equivalent to a 2-fold to 1000-fold (e.g., 2-fold to 100-fold, 10-fold to 1000-fold) reduction in the standard of care dose of a recombinant or purified RSV protein vaccine, wherein the anti-RSV antigen antibody titer produced in the subject is equivalent to an anti-RSV antigen antibody titer produced in a control subject administered the standard of care dose of a recombinant or purified RSV protein vaccine, or a live attenuated or inactivated RSV vaccine, or an RSV VLP vaccine.

In some embodiments, the effective amount of an RSV RNA (e.g., mRNA) vaccine is a dose equivalent to a 2 to 1000-, 2 to 900-, 2 to 800-, 2 to 700-, 2 to 600-, 2 to 500-, 2 to 400-, 2 to 300-, 2 to 200-, 2 to 100-, 2 to 90-, 2 to 80-, 2 to 70-, 2 to 60-, 2 to 50-, 2 to 40-, 2 to 30-, 2 to 20-, 2 to 10-, 2 to 9-, 2 to 8-, 2 to 7-, 2 to 6-, 2 to 5-, 2 to 4-, 2 to 3-, 3 to 1000-, 3 to 900-, 3 to 800-, 3 to 700-, 3 to 600-, 3 to 500-, 3 to 400-, 3 to 3 to 00-, 3 to 200-, 3 to 100-, 3 to 90-, 3 to 80-, 3 to 70-, 3 to 60-, 3 to 50-, 3 to 40-, 3 to 30-, 3 to 20-, 3 to 10-, 3 to 9-, 3 to 8-, 3 to 7-, 3 to 6-, 3 to 5-, 3 to 4-, 4 to 1000-, 4 to 900-, 4 to 800-, 4 to 700-, 4 to 600-, 4 to 500-, 4 to 400-, 4 to 300-, 4 to 200-, 4 to 100-, 4 to 90-, 4 to 80-, 4 to 70-, 4 to 60-, 4 to 50, 4 to 40-, 4 to 30-, 4 to 20-, 4 to 10-, 4 to 9-, 4 to 8-, 4 to 7-, 4 to 6-, 4 to 5-, 4 to 4-, 5 to 1000-, 5 to 900-, 5 to 800-, 5 to 700-, 5 to 600-, 5 to 500-, 5 to 400-, 5 to 300-, 5 to 200-, 5 to 100-, 5 to 90-, 5 to 80-, 5 to 70-, 5 to 60-, 5 to 50-, 5 to 40-, 5 to 30-, 5 to 20-, 5 to 10-, 5 to 9-, 5 to 8, 5 to 7-, 5 to 6-, 6 to 1000-, 6 to 900-, 6 to 800-, 6 to 700-, 6 to 600-, 6 to 500-, 6 to 400-, 6 to 300-, 6 to 200-, 6 to 100-, 6 to 90-, 6 to 80-, 6 to 70-, 6 to 60-, 6 to 50-, 6 to 40-, 6 to 30-, 6 to 20-, 6 to 10-, 6 to 9-, 6 to 8-, 6 to 7-, 7 to 1000-, 7 to 900-, 7 to 800-, 7 to 700-, 7 to 600-, 7 to 500-, 7 to 400-, 7 to 300-, 7 to 200-, 7 to 100-, 7 to 90-, 7 to 80-, 7 to 70-, 7 to 60-, 7 to 50-, 7 to 40-, 7 to 30-, 7 to 20-, 7 to 10-, 7 to 9-, 7 to 8-, 8 to 1000-, 8 to 900-, 8 to 800-, 8 to 700-, 8 to 600-, 8 to 500-, 8 to 400-, 8 to 300-, 8 to 200-, 8 to 100-, 8 to 90-, 8 to 80-, 8 to 70-, 8 to 60-, 8 to 50-, 8 to 40-, 8 to 30-, 8 to 20-, 8 to 10-, 8 to 9-, 9 to 1000-, 9 to 900-, 9 to 800-, 9 to 700-, 9 to 600-, 9 to 500-, 9 to 400-, 9 to 300-, 9 to 200-, 9 to 100-, 9 to 90-, 9 to 80-, 9 to 70-, 9 to 60-, 9 to 50-, 9 to 40-, 9 to 30-, 9 to 20-, 9 to 10-, 10 to 1000-, 10 to 900-, 10 to 800-, 10 to 700-, 10 to 600-, 10 to 500-, 10 to 400-, 10 to 300-, 10 to 200-, 10 to 100-, 10 to 90-, 10 to 80-, 10 to 70-, 10 to 60-, 10 to 50-, 10 to 40-, 10 to 30-, 10 to 20-, 20 to 1000-, 20 to 900-, 20 to 800-, 20 to 700-, 20 to 600-, 20 to 500-, 20 to 400-, 20 to 300-, 20 to 200-, 20 to 100-, 20 to 90-, 20 to 80-, 20 to 70-, 20 to 60-, 20 to 50-, 20 to 40-, 20 to 30-, 30 to 1000-, 30 to 900-, 30 to 800-, 30 to 700-, 30 to 600-, 30 to 500-, 30 to 400-, 30 to 300-, 30 to 200-, 30 to 100-, 30 to 90-, 30 to 80-, 30 to 70-, 30 to 60-, 30 to 50-, 30 to 40-, 40 to 1000-, 40 to 900-, 40 to 800-, 40 to 700-, 40 to 600-, 40 to 500-, 40 to 400-, 40 to 300-, 40 to 200-, 40 to 100-, 40 to 90-, 40 to 80-, 40 to 70-, 40 to 60-, 40 to 50-, 50 to 1000-, 50 to 900-, 50 to 800-, 50 to 700-, 50 to 600-, 50 to 500-, 50 to 400-, 50 to 300-, 50 to 200-, 50 to 100-, 50 to 90-, 50 to 80-, 50 to 70-, 50 to 60-, 60 to 1000-, 60 to 900-, 60 to 800-, 60 to 700-, 60 to 600-, 60 to 500-, 60 to 400-, 60 to 300-, 60 to 200-, 60 to 100-, 60 to 90-, 60 to 80-, 60 to 70-, 70 to 1000-, 70 to 900-, 70 to 800-, 70 to 700-, 70 to 600-, 70 to 500-, 70 to 400-, 70 to 300-, 70 to 200-, 70 to 100-, 70 to 90-, 70 to 80-, 80 to 1000-, 80 to 900-, 80 to 800-, 80 to 700-, 80 to 600-, 80 to 500-, 80 to 400-, 80 to 300-, 80 to 200-, 80 to 100-, 80 to 90-, 90 to 1000-, 90 to 900-, 90 to 800-, 90 to 700-, 90 to 600-, 90 to 500-, 90 to 400-, 90 to 300-, 90 to 200-, 90 to 100-, 100 to 1000-, 100 to 900-, 100 to 800-, 100 to 700-, 100 to 600-, 100 to 500-, 100 to 400-, 100 to 300-, 100 to 200-, 200 to 1000-, 200 to 900-, 200 to 800-, 200 to 700-, 200 to 600-, 200 to 500-, 200 to 400-, 200 to 300-, 300 to 1000-, 300 to 900-, 300 to 800-, 300 to 700-, 300 to 600-, 300 to 500-, 300 to 400-, 400 to 1000-, 400 to 900-, 400 to 800-, 400 to 700-, 400 to 600-, 400 to 500-, 500 to 1000-, 500 to 900-, 500 to 800-, 500 to 700-, 500 to 600-, 600 to 1000-, 600 to 900-, 600 to 800-, 600 to 700-, 700 to 1000-, 700 to 900-, 700 to 800-, 800 to 1000-, 800 to 900-, or 900 to 1000-fold reduction in the standard of care dose of a recombinant RSV protein vaccine. In some embodiments, such as the foregoing, the anti-RSV antigen antibody titer produced in the subject is equivalent to an anti-RSV antigen antibody titer produced in a control subject administered the standard of care dose of a recombinant or purified RSV protein vaccine, or a live attenuated or inactivated RSV vaccine, or an RSV VLP vaccine. In some embodiments, the effective amount is a dose equivalent to (or equivalent to an at least) 2-, 3-, 4-, 5-, 6-, 7-, 8-, 9-, 10-, 20-, 30-, 40-, 50-, 60-, 70-, 80-, 90-, 100-, 110-, 120-, 130-, 140-, 150-, 160-, 170-, 1280-, 190-, 200-, 210-, 220-, 230-, 240-, 250-, 260-, 270-, 280-, 290-, 300-, 310-, 320-, 330-, 340-, 350-, 360-, 370-, 380-, 390-, 400-, 410-, 420-, 430-, 440-, 450-, 460-, 470-, 480-, 490-, 500-, 510-, 520-, 530-, 540-, 550-, 560-, 5760-, 580-, 590-, 600-, 610-, 620-, 630-, 640-, 650-, 660-, 670-, 680-, 690-, 700-, 710-, 720-, 730-, 740-, 750-, 760-, 770-, 780-, 790-, 800-, 810-, 820-, 830-, 840-, 850-, 860-, 870-, 880-, 890-, 900-, 910-, 920-, 930-, 940-, 950-, 960-, 970-, 980-, 990-, or 1000-fold reduction in the standard of care dose of a recombinant RSV protein vaccine. In some 5 embodiments, such as the foregoing, an anti-RSV antigen antibody titer produced in the subject is equivalent to an anti-RSV antigen antibody titer produced in a control subject administered the standard of care dose of a recombinant or purified RSV protein vaccine, or a live attenuated or inactivated RSV vaccine, or an RSV VLP vaccine.

In some embodiments, the effective amount of an RSV RNA (e.g., mRNA) vaccine is a total dose of 50-1000 g. In some embodiments, the effective amount of an RSV RNA (e.g., mRNA) vaccine is a total dose of 50-1000, 50-900, 50-800, 50-700, 50-600, 50-500, 50-400, 50-300, 50-200, 50-100, 50-90, 50-80, 50-70, 50-60, 60-1000, 60-900, 60-800, 60-700, 60-600, 60-500, 60-400, 60-300, 60-200, 60-100, 60-90, 60-80, 60-70, 70-1000, 70-900, 70-800, 70-700, 70-600, 70-500, 70-400, 70-300, 70-200, 70-100, 70-90, 70-80, 80-1000, 80-900, 80-800, 80-700, 80-600, 80-500, 80-400, 80-300, 80-200, 80-100, 80-90, 90-1000, 90-900, 90-800, 90-700, 90-600, 90-500, 90-400, 90-300, 90-200, 90-100, 100-1000, 100-900, 100-800, 100-700, 100-600, 100-500, 100-400, 100-300, 100-200, 200-1000, 200-900, 200-800, 200-700, 200-600, 200-500, 200-400, 200-300, 300-1000, 300-900, 300-800, 300-700, 300-600, 300-500, 300-400, 400-1000, 400-900, 400-800, 400-700, 400-600, 400-500, 500-1000, 500-900, 500-800, 500-700, 500-600, 600-1000, 600-900, 600-900, 600-700, 700-1000, 700-900, 700-800, 800-1000, 800-900, or 900-1000 g. In some embodiments, the effective amount of an RSV RNA (e.g., mRNA) vaccine is a total dose of 50, 100, 150, 200, 250, 300, 350, 400, 450, 500, 550, 600, 650, 700, 750, 800, 850, 900, 950 or 1000 g. In some embodiments, the effective amount is a dose of 25-500 g administered to the subject a total of two times. In some embodiments, the effective amount of an RSV RNA (e.g., mRNA) vaccine is a dose of 25-500, 25-400, 25-300, 25-200, 25-100, 25-50, 50-500, 50-400, 50-300, 50-200, 50-100, 100-500, 100-400, 100-300, 100-200, 150-500, 150-400, 150-300, 150-200, 200-500, 200-400, 200-300, 250-500, 250-400, 250-300, 300-500, 300-400, 350-500, 350-400, 400-500 or 450-500 g administered to the subject a total of two times. In some embodiments, the effective amount of an RSV RNA (e.g., mRNA) vaccine is a total dose of 25, 50, 100, 150, 200, 250, 300, 350, 400, 450, or 500 g administered to the subject a total of two times.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

$$Efficacy = ( ARU - ARV ) / ARU \times 100 ; and$$

$$Efficacy = ( 1 - RR ) \times 10 \; 0 .$$

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

$$Effectiveness = ( 1 - OR ) \times 10 \; 0 .$$

In some embodiments, efficacy of the RSV vaccine is at least 60% relative to unvaccinated control subjects. For example, efficacy of the RSV vaccine may be at least 65%, at least 70%, at least 75%, at least 80%, at least 85%, at least 95%, at least 98%, or 100% relative to unvaccinated control subjects.

### Sterilizing Immunity.

Sterilizing immunity refers to a unique immune status that prevents effective pathogen infection into the host. In some embodiments, the effective amount of an RSV vaccine of the present disclosure is sufficient to provide sterilizing immunity in the subject for at least 1 year. For example, the effective amount of an RSV vaccine of the present disclosure is sufficient to provide sterilizing immunity in the subject for at least 2 years, at least 3 years, at least 4 years, or at least 5 years. In some embodiments, the effective amount of an RSV vaccine of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

### Detectable Antigen.

In some embodiments, the effective amount of an RSV vaccine of the present disclosure is sufficient to produce detectable levels of RSV antigen as measured in serum of the subject at 1-72 hours post administration.

### Titer.

An antibody titer is a measurement of the amount of antibodies within a subject, for example, antibodies that are specific to a particular antigen (e.g., an anti-RSV antigen).

Antibody titer is typically expressed as the inverse of the greatest dilution that provides a positive result. Enzyme-linked immunosorbent assay (ELISA) is a common assay for determining antibody titers, for example.

In some embodiments, the effective amount of an RSV vaccine of the present disclosure is sufficient to produce a 1,000-10,000 neutralizing antibody titer produced by neutralizing antibody against the RSV antigen as measured in serum of the subject at 1-72 hours post administration. In some embodiments, the effective amount is sufficient to produce a 1,000-5,000 neutralizing antibody titer produced by neutralizing antibody against the RSV antigen as measured in serum of the subject at 1-72 hours post administration. In some embodiments, the effective amount is sufficient to produce a 5,000-10,000 neutralizing antibody titer produced by neutralizing antibody against the RSV antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-RSV antigen antibody titer produced in the subject is increased by at least 1 log relative to a control. For example, an anti-RSV antigen antibody titer produced in the subject may be increased by at least 2, 3, 4, 5, 6, 7, 8, 9 or 10 log relative to a control.

In some embodiments, an anti-RSV antigen antibody titer produced in the subject is increased at least 2 times relative to a control. For example, an anti-RSV antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a live attenuated RSV vaccine, an inactivated RSV vaccine, or a protein subunit RSV vaccine.

### EXAMPLES

### Example 1: Cotton Rat Immunogenicity and Efficacy

In this example, assays were carried out to test the immunogenicity and efficacy of mRNA/LNP vaccines in the cotton rat RSV challenge model. More specifically, female Sigmodon hispidus cotton rats were used and immunizations began at 6-7 weeks of age. The mRNA vaccines used were generated and formulated in lipid nanoparticles (e.g the LNP comprises 58 mole % (13Z,16Z)—N,N-dimethyl-2-nonylhenicosa-12,15-dien-1-amine, 30 mole % cholesterol, 10 mole % DSPC, and 2 mole % PEG-2000 DMG). The mRNA vaccines evaluated in this study included:
MRK-04 membrane-bound DS-Cav1 (stabilized prefusion F protein) (SEQ ID NO: 90)
MRK-04_nopolyA_3mut membrane-bound DS-Cav1 (stabilized prefusion F protein) (SEQ ID NO: 92)
mVRC-1 (v2) membrane-bound single chain sc9 mDS-Cav1, A149C, Y458C (stabilized prefusion F protein) (SEQ ID NO: 21)
mLZF-111 membrane-bound single chain mDS-Cav1, D486C, D489C (stabilized prefusion F protein) (SEQ ID NO: 74)
Groups of 8 cotton rats were immunized intramuscularly with 100 μL of vaccine, delivered with 50 μL injections into each quadriceps. The groups were vaccinated with the following vaccines:

TABLE 1
Vaccine Formulations Tested for Cotton Rats

| | | SEQ ID | Conc | Dose |
|---|---|---|---|---|
| Group | Vaccine | NO: | (μg/ml) | (μg) |
| 1 | None | n/a | NA | NA |
| 2 | MRK-04, I.M. | 90 | 250 | 25 |
| 3 | MRK-04_nopolyA_3mut, I.M. | 92 | 250 | 25 |
| 4 | MRK-04_nopolyA_3mut, I.M. | 92 | 50 | 5 |
| 5 | MRK-04_nopolyA_3mut, | 92 | 10 | 1 |
| | I.M.. | | | |
| 6 | mVRC-1 (v2), I.M. | 21 | 250 | 25 |
| 7 | mVRC-1 (v2), I.M. | 21 | 50 | 5 |
| 8 | mVRC-1 (v2), I.M. | 21 | 10 | 1 |
| 9 | mLZF-111, I.M. | 74 | 250 | 25 |
| 10 | mLZF-111, I.M. | 74 | 50 | 5 |
| 11 | mLZF-111, I.M. | 74 | 10 | 1 |

The animals were immunized on day 0 and day 28 of the experiment. On days 28 and 56, blood was drawn from each animal and used for serological assays. On day 56, the cotton rats were challenged intranasally with 1×10^{5.5} PFU RSV A2. Four days post inoculation animals were sacrificed by CO₂ inhalation and lung (left lobes) and nasal turbinates were removed and homogenized in 10 volumes of Hanks Balanced Salt Solution (Lonza) containing SPG on wet ice. The samples were clarified by centrifugation at 2000 rpm for 10 minutes, aliquoted, flash frozen, and immediately stored frozen at −70° C.

### A. RSV Neutralization Assay:

Cotton rat sera from each animal was evaluated for neutralization of RSV-A (Long strain) using the following procedures:
1. All sera samples were heat inactivated by placing in dry bath incubator set at 56° C. for 30 minutes. Samples and control sera were then diluted 1:3 in virus diluent (2% FBS in EMEM) and duplicate samples were added to an assay plate and serially diluted.
2. RSV-Long stock virus was removed from the freezer and quickly thawed in 37° C. water bath. Viruses were diluted to 2000 pfu/mL in virus diluent
3. 50 μL of diluted virus was added to each well of the 96-well plate, with the exception of one column of cells, which used as a “no-virus” control
4. HEp-2 cells were trypsinized, washed, resuspended at 1.5×10⁵ cells/ml in virus diluent, and 100 mL of the suspended cells were added to each well of the 96-well plate. The plates were then incubated for 72 hours at 37° C., 5% CO₂.
5. Following the 72 hour incubation, the cells were washed with PBS, and fixed using 80% acetone dissolved in PBS for 10-20 minutes at 16-24° C. The fixative was removed and the plates were allowed to air-dry.
6. Plates were then washed thoroughly with PBS+0.05% Tween™ (polysorbate). The detections monoclonal antibodies, 143-F3-1B8 and 34C9 were diluted to 2.5 μg/mL in assay diluent (1% BSA-PBS-0.1% Tween), and 50 μL of the diluted antibodies were added to each well of the 96-well plate. The plates were then incubated in a humid chamber at 16-24° C. for 60-75 minutes on rocker.
7. Following the incubation, the plates were thoroughly washed.
8. Biotinylated horse anti-mouse IgG was diluted 1:200 in assay diluent and added to each well of the 96-well plate. Plates were incubated as above and washed.
9. A cocktail of IRDye 800CW Streptavidin (1:1000 final dilution), Sapphire 700 (1:1000 dilution) and 5 mM DRAQ5 solution (1:10,000 dilution) was prepared in assay diluent and 50 mL of the cocktail was added to each well of the 96-well plate. Plates were incubated as above in the dark, washed, and allowed to air dry.
10. Plates were then read using an Aerius Imager. Serum neutralizing titers were then calculated using a 4 parameter curve fit in Graphpad Prism.

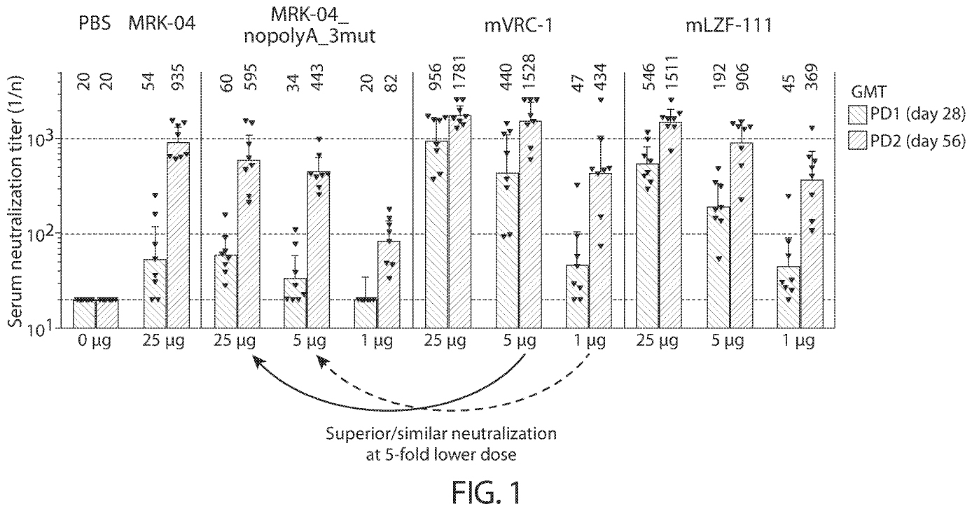

The titers determined post dose 1 (day 28) and post dose 2 (day 56) are shown in FIG. 1. It was found that the neutralizing titers were elicited in a dose dependent manner for all mRNA vaccines. All mRNA vaccines resulted in increased titers after a second dose regardless of the dose evaluated. Both mVRC-1 (v2) and mLVF111 induced higher titers then MRK-04 and MRK-04_nopolyA_3mut demonstrating superior or similar serum neutralizing titers at a 5-fold lower dose.

B. Competition alphaLISA:

The immune response to specific epitopes on RSV F-protein for neutralizing antibodies was characterized. The antigenic site II is the binding site for palivizumab, a monoclonal antibody developed for the prevention of lower respiratory infection with RSV in at risk infants and toddlers. Antigenic site Ø is a binding site for more potent neutralizing antibodies that are elicited by natural infection with RSV. Additionally, we have generated an antibody (4D7) that targets site I, an epitope not presented in the prefusion conformation. Therefore, in contrast to D25, elicitation of 4D7-competing antibodies would suggest the in vivo generation of postF-like proteins. A competition alphaLISA was developed to characterize the antigenic site Ø, antigenic site I and antigenic site II response to the various mRNA-based vaccines.

To measure competing antibody titers, 10 μl of samples serially diluted in HiBlock buffer (PerkinElmer) are placed in a 384 well alphaLISA plate. Diluted samples are mixed with 5 μl of AlphaLISA acceptor beads (100 μg/ml) that has been previously conjugated to a prefusion-stabilized RSV F protein (DS-Cav1) or a postfusion RSV F protein (RSV F wt). After 30 min incubation at room temperature, 10 μl of biotinylated D25, palimizumab, or 4D7 antibody diluted in Hiblock buffer is added to every well. After additional 30 min incubation, 25 μl of streptavidin-donor beads (20 μg/ml) in HiBlock buffer is added to each well and incubated for 30 min in the dark. Plate is then read on an EnVision_Alpha Reader (615 nm detection).

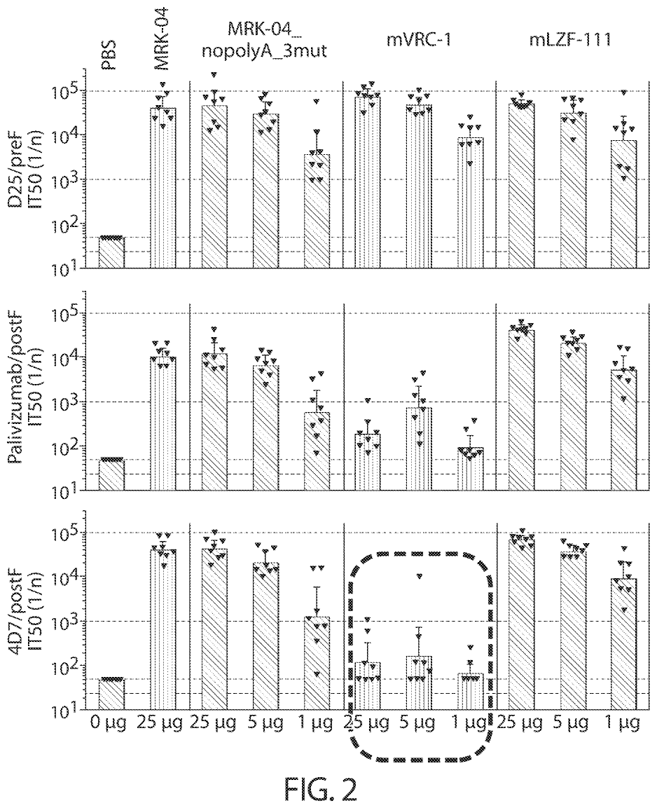

The palivizumab, D25 and 4D7 competing antibody titers measured on Day 56 (4 weeks PD2) are presented in FIG. 2. The competition data revealed that mVRC-1 (v2) induced lower levels of 4D7-postfusion F competing antibodies, while D25-prefusion titers and palivizumab titers are not affected. This different competition profile correlates with mVRC-1 (v2) mRNA expressing a more prefusion stabilized protein then MRK-04_nopolyA_3mut and mLZF-111.

### C. Cotton Rat Challenge Results

Procedures for measuring RSV titers in the cotton rat Lung and nose homogenates are described below. Lung and nose homogenates were clarified by centrifugation and diluted 1:10 and 1:100 in EMEM. Confluent HEp-2 monolayers were infected in duplicates with 50 μl per well starting with undiluted (neat) samples followed by diluted homogenates in 24-well plates. After one hour incubation at 37° C. in a 5% CO₂ incubator, wells were overlaid with 0.75% methylcellulose medium and plates restored into the 37° C. incubator. After 4 days of incubation the overlay was removed and the cells were fixed with 0.1% crystal violet stain for one hour, then rinsed, and air-dried. Plaques were counted and virus titers were expressed as plaque forming units per gram of tissue.

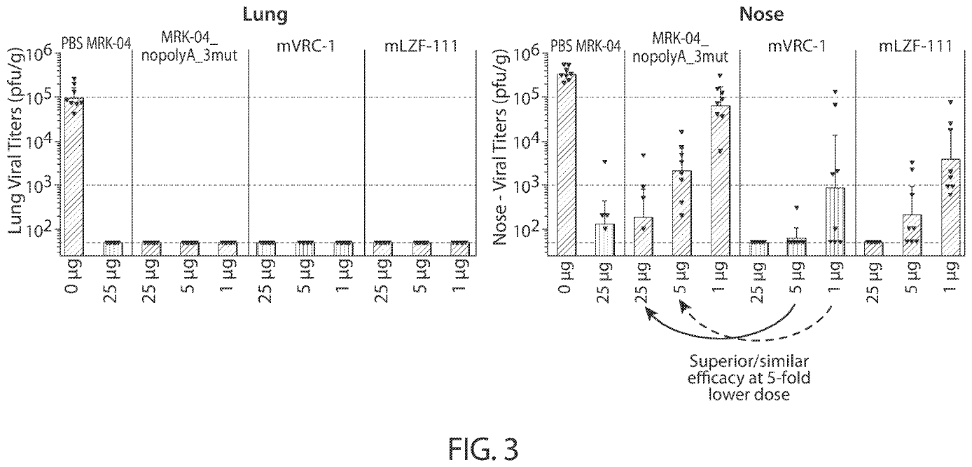

To assess vaccine-mediated protection, viral titers were measured in lung and nose 5 days after challenge. All mRNA vaccines achieved total protection in the lung, but mVRC-1 (v2) and mLZF-111 showed improved protection in the nose, demonstrating superior or similar efficacy to MRK-04 and MRK-04_nopolyA_3mut, at a 5-fold lower dose (FIG. 3).

### Example 2: African Green Monkey Immunogenicity and Efficacy

In this example, assays were carried out to test the immunogenicity and efficacy of mRNA/LNP vaccines in the African Green Monkey RSV challenge model.

More specifically, male and female adult African Green Monkeys with body weights ranging from 1.6 to 2.65 kg, which were confirmed to be RSV-negative by neutralizing antibody titer, were used. The mRNA vaccines used were generated and formulated in lipid nanoparticles (e.g the LNP comprises 58 mole % (13Z,16Z)—N,N-dimethyl-2-nonylhenicosa-12,15-dien-1-amine, 30 mole % cholesterol, 10 mole % DSPC, and 2 mole % PEG-2000 DMG). The mRNA vaccines evaluated in this study included:
MRK-04_nopolyA_3mut membrane-bound DS-Cav1 (stabilized prefusion F protein) (SEQ ID NO: 92)
mVRC-1 (v2) membrane-bound single chain sc9 mDS-Cav1, A149C, Y458C (stabilized prefusion F protein) (SEQ ID NO: 21)
mLZF-111 membrane-bound single chain mDS-Cav1, D486C, D489C (stabilized prefusion F protein) (SEQ ID NO: 74)

Groups of 4 African Green Monkeys were immunized intramuscularly with 500 μL of vaccine into one deltoid. The groups were vaccinated with the following vaccines as out in

TABLE 2
Vaccine Formulations Tested for Immunogenicity
in African Green Monkeys

| Group | Vaccine | Conc (μg/ml) | Dose (μg) |
|---|---|---|---|
| 1 | MRK-04_nopolyA_3mut, I.M. | 50 | 25 |
| 2 | MRK-04_nopolyA_3mut, I.M. | 10 | 5 |
| 3 | mVRC-1 (v2) | 50 | 25 |
| 4 | mVRC-1 (v2) | 10 | 5 |
| 5 | mLZF-111 | 50 | 25 |
| 6 | mLZF-111 | 10 | 5 |
| 7 | RSV A2 5.5log10pfu, I.N. | NA | NA |
| 8 | None | NA | NA |

The animals were immunized on day 0, day 28, and day 56 of the experiment. On days 0, 14, 28, 42, 56, and 70, blood was drawn from each animal and used for serological assays. On day 70, the African Green Monkeys were challenged intranasally with 1×10^{5.5} PFU RSV A2. Nasopharyngeal swabs were collected on days 1-14 post challenge, and lung lavage samples were collected on days 3, 5, 7, 9, 12 and 14 post challenge to test for viral replication.

### A. RSV Neutralization Assay:

Monkey sera from each animal were evaluated for neutralization of RSV-A (Long strain) as described above. The NT₅₀ titers determined post dose 1 and post dose 2 are shown in FIG. 4. Titers were seen to increase after each dose all groups receiving mRNA vaccines. The GMTs obtained with mRNA vaccines at week 10 (2 weeks post-dose 3) were 1 to 2 orders of magnitude higher than in the animals that received RSV A2 depending on the dose and mRNA being tested. Serum samples from mVRC-1 (v2) immunized animals exhibited the highest neutralization titers, demonstrating a five-fold higher potency relative to MRK-04_nopolyA_3mut.

### B. Competition ELISA

Competition ELISA titers were determined for palivizumab, D25 and 4D7 to characterize the antigenic site Ø, antigenic site I and antigenic site II response to the various mRNA-based vaccines as described above.

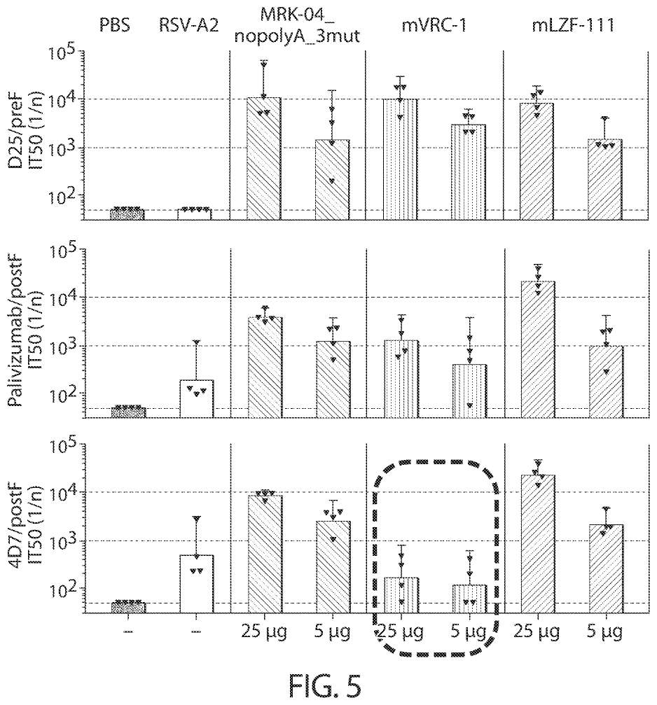

The palivizumab, D25 and 4D7 competing antibody titers measured at week 10 (2 weeks PD3) are presented in FIG. 5. The competition data revealed that mVRC-1 (v2) induced lower levels of 4D7-postfusion F competing antibodies, while D25-prefusion titers and palivizumab titers are not affected. This different competition profile correlates with mVRC-1 (v2) mRNA expressing a more prefusion stabilized protein then MRK-04_nopolyA_3mut and mLZF-111.

### C. African Green Monkey Challenge Results

As mentioned above, in order to evaluate vaccine efficacy African Green Monkeys were challenged intranasally with 1×10^{5.5} PFU RSV A2 on day 70 post vaccination and nasopharyngeal swabs and lung lavage samples were collected post challenge to test for the presence of virus.

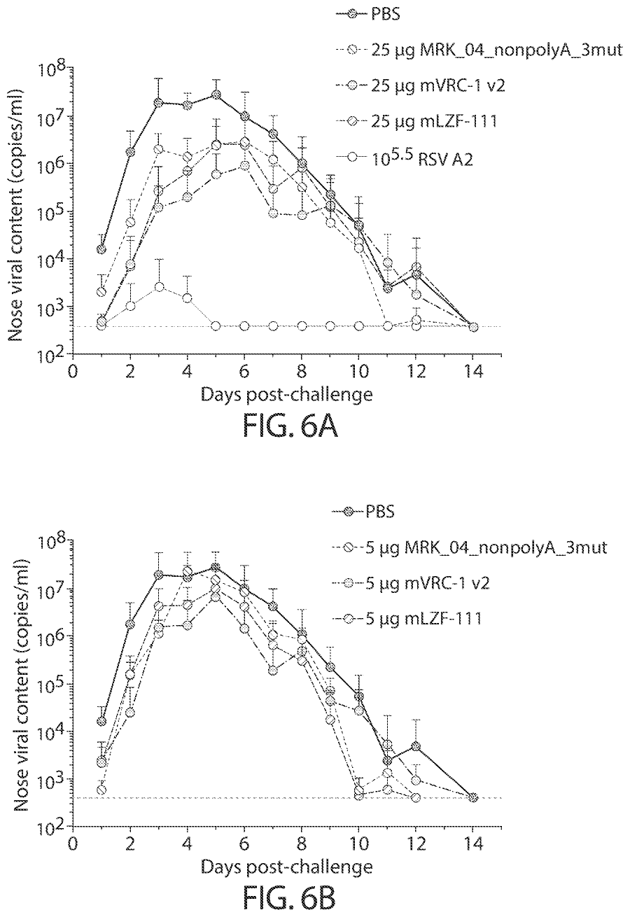

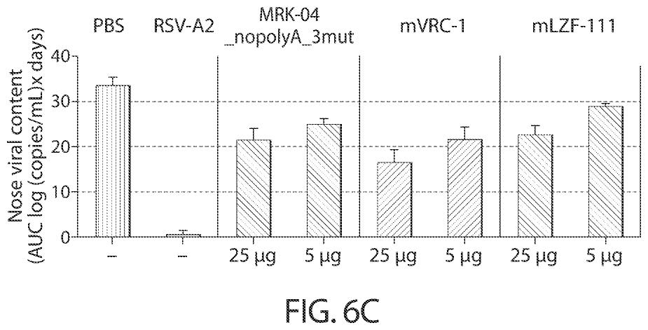

In order to measure RSV titers in the African Green Monkey lung lavage samples a viral plaque assay procedure for measuring viral titers was followed as outlined below. Briefly, samples were diluted and added in duplicate to 24-well plates containing confluent HEp-2 cell monolayers. The plates were incubated at 37° C. for one hour. Following the one hour incubation, sample inoculum was aspirated and 1 ml of overlay containing 0.75% methylcellulose was added. The plates were incubated at 37° C. for 5 days. Following the 5 day incubation, the cells were fixed and stained with crystal violet/glutaraldehyde solution. Plaques were counted and titers were expressed as pfu/ml. Analysis of viral content in bronchoalveolar lavage (BAL) fluid (FIGS. 6A-6C) revealed that only mVRC-1 (v2) (25 μg) conferred total protection in the lung, and it afforded the best protection at a lower dose (5 μg).

In order to measure RSV titers in the African Green Monkey nasopharyngeal swabs an RSV RT-qPCR assay to detect RSV A was carried out as follows:
1) Equipment and Materials:
   A. Equipment
      1. Stratagene Mx3005P Real Time PCR system and MxPro Software
      2. Jouan GR422 centrifuge or equivalent
      3. Jouan Plate carriers or equivalent
   B. Reagents
      1. Quantitect® Probe Rt-PCR kit (1000) catalog #204445
      2. Water, Molecular Biology Grade DNAase-free and Protease free, 5 Prime, catalog #2900136
      3. TE buffer, 10 mM Tris 1 mM EDTA ph 8.0, Fisher Bioreagents, catalog #BP2473-100
      4. Viral primers: RSV A Forward and Reverse primers, Sigma custom, HPLC purified. Primer stocks are reconstituted to 100 uM in Molecular grade water and stored at −20° C.
      5. RSV dual labeled probe, Sigma custom, HPLC purified. Probe stocks are reconstituted to 100 uM in TE buffer and stored at −20° C. protected from light.
      6. RSV A standard were generated in-house and stored at −20° C. Standards for the assay were generated by designing primer pairs to the N gene of RSV A. The product length for the RSV A standard is 885 bp. QIAGEN OneStep RT-PCR was used to generate this standard.

TABLE 3
Primer Sequences

| Primers | Sequences |
|---|---|
| RSV A F | 5′ CTC AAT TTC CTC ACT TCT CCA GTG T |
| N gene | (SEQ ID NO: 93) |
| RSV A R | 5′ CTT GAT TCC TCG GTG TAC CTC TGT |
| N gene | (SEQ ID NO: 94) |
| RSV A | 5′FAM-TCC CAT TAT GCC TAG GCC AGC |
| FAM N | AGC A (BHQ1) |
| gene | (SEQ ID NO: 95) |

7. Promega, Maxwell®16 Viral Total Nucleic Acid Purification Kit (Product #AS1150
C. Supplies
   1. Stratagene Optical cap 8× strip, catalog #401425
   2. Stratagene Mx3000P 96 well plates, skirted, catalog #401334
   3. ART filtered pipet tips
2) RT-PCR Reactions and set up
   A. Preparation of Complete Master Mix
      1. Prepare complete Master Mix following the set up below for a final reaction volume of 50 μL. The following table is volume per well. Final primer concentration is 300 nM and final probe concentration is 200 nM.

TABLE 4
Reagents

| Reagent | μL |
|---|---|
| 2X Master Mix | 25 |
| RSV A F 100 uM | 0.2 |
| RSV A R 100 uM | 0.2 |
| RSV A FAM 100 uM | 0.1 |
| RT enzyme mix | 0.5 |
| Water | 19 |

2. Add 45 μL of complete master mix to each well. Cover plate with plate cover and wrap in aluminum foil to protect from light.
B. Preparation of Standard curve
   1. Remove standard from −20° C.
   2. Dilute standards to final concentrations of 1e6 copy/5 μL to 1copy/5 μL using 10-fold dilutions.
C. Sample preparation
   1. Nasopharyngeal swab and lung lavage samples are prepared for the RT-PCR reaction using the Maxwell® 16 Viral Total Nucleic Acid Purification Kit (Promega, product #AS1150)
   2. 200 μL of sample is extracted following the manufactures protocol and eluted into 50 μL to be used in PCR reactions.
D. Additions of samples
   1. Add 5 μL of extracted samples to appropriate wells. After addition of samples, carefully cap sample wells before adding standard curves.
   2. Add 5 μL of diluted standard to appropriate wells and cap.
   3. Add 5 μL of molecular grade water to No Template Control (NTC) wells.
   4. Wrap plates in aluminum foil and transfer plates to centrifuge.
   5. Spin plates for 2 mins at 100 rpm to pull down any samples or master mix that may be on the sides of well.
   6. Wrap plates in aluminum foil and transfer to Stratagene instrument.
E. Thermo cycler: Stratagene MX 3005P
   1. Place plates in Stratagene Mx3005P and set thermal profile conditions to:

TABLE 5
PCR Conditions

| Step | Time | | Temperature |
|---|---|---|---|
| Reverse Transcription | 30 | min | 50 |
| PCR intial activation step | 15 | min | 95 |
| 2-step cycling: | | | |
| Denaturation | 16 | sec | 94 |
| Combined annealing/extension | 60 | sec | 62 |

| | |
|---|---|
| Number of cycles | 40 |

2. Analyze results using the Stratagene Mx3005p software

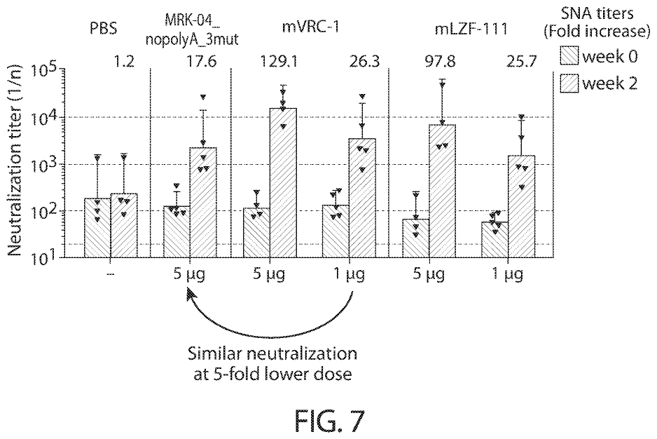

The mean RNA copy number detected in the nose samples are presented in FIG. 7. The protective effect of all mRNA-based vaccines was less apparent in the nose, but again mVRC-1 (v2) demonstrated a 5-fold higher efficacy over MRK-04_nopolyA_3mut. mLZF-111 demonstrated decreased protection efficacy in lung and nose of AGMs relative to MRK-04_nopolyA_3mut.

### Example 3: Immunogenicity in RSV-Experienced African Green Monkeys

The immunogenicity of mRNA vaccines formulated in LNP was tested in RSV-experienced African Green Monkeys.

Healthy adult, African Green Monkeys of either sex (n=4 or 5/group), with body weights ranging from 2.85 to 4.65 kg, that were confirmed to be RSV seropositive by ELISA and neutralizing antibody titers, were selected for the study. The pool of animals selected for this study had been experimentally infected with RSV in previous vaccine studies and were distributed across study groups based on their pre study RSV neutralization titers so that all groups would have similar group GMTs at study start. RSV experienced animals provide a model of immune memory recall response to vaccination that may reflect the responses that can be anticipated in seropositive human adults, with the caveat that the antibody response in AGMs following RSV exposure is more biased towards postfusion F protein epitopes than the human immune repertoire.

A single vaccine dose was administered to each animal at week 0 by the intramuscular (IM) route. A control group receiving only PBS was also included in the study design. Vaccines were administered as described in Table 6. After vaccination, the animals were observed daily for any changes at the inoculation site or other changes in activity or feeding habits that might indicate an adverse reaction to the vaccine but none were noted. Serum samples were collected for assessment of RSV neutralizing antibody titers, as well as palivizumab (site II), D25 (site Ø) and 4D7 (site I) competing antibody titers. PBMC samples were collected to assess cell mediated immune responses.

TABLE 6
Vaccine Formulations Tested for Immunogenicity
in RSV Seropositive African Green Monkeys

| Group | Vaccine | Conc (μg/ml) | Dose (μg) |
|---|---|---|---|
| 1 | MRK-04_nopolyA_3mut, I.M. | 10 | 5 |
| 2 | mVRC-1 (v2), I.M. | 10 | 5 |
| 3 | mVRC-1 (v2), I.M | 2 | 1 |
| 4 | mLZF-111 | 10 | 5 |
| 5 | mLZF-111 | 2 | 1 |
| 6 | None (PBS) | NA | NA |

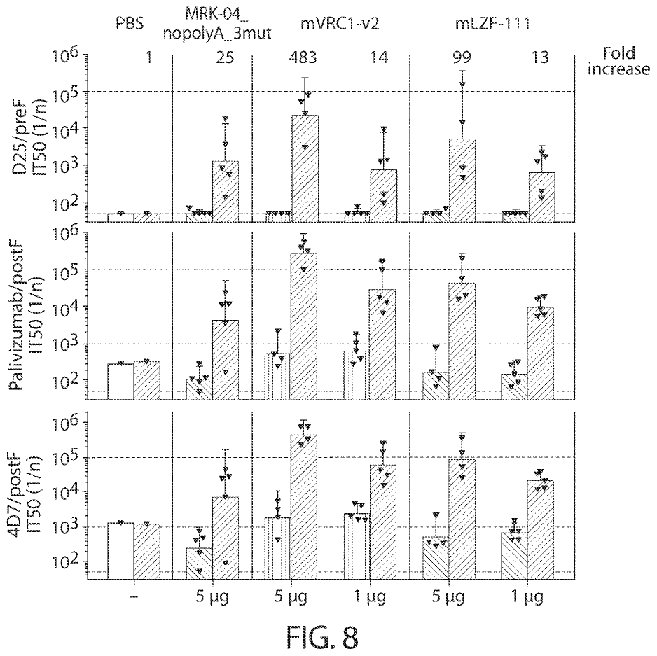

Individual animal NT₅₀ titers were measured in serum samples collected at baseline and 2 weeks post vaccination using methods described above and the results are shown in FIG. 8. All vaccines were found to be highly immunogenic as demonstrated by the increase in levels of serum antibodies binding RSV F proteins (both prefusion and postfusion RSV F, data not shown) and increases in serum neutralizing antibody levels. mVRC-1 (v2) induced the highest boost in neutralizing titers (>100 fold at the highest dose), and exhibited similar potency at a 5-fold lower dose relative to MRK-04_nopolyA_3mut. Similarly, mLZF-111 also demonstrated increased potency relative to MRK-04_nopolyA_3mut. No increase in titers was observed in the PBS control group. As this study is still ongoing the durability of these immune responses will continue to be evaluated over the next several months.

To evaluate the quality of the boosted responses in the vaccinated animals, palivizumab (site II), D25 (site Ø) and 4D7 (site I) competing antibody titers were determined in serum collected at 2 weeks post vaccination (FIG. 8). As described above, antigenic site II is a neutralization epitope found on both the prefusion and the postfusion conformation of the F protein, site Ø is a prefusion specific neutralization epitope and 4D7 is a postfusion specific neutralization epitope. Due to the baseline immune bias to the postfusion conformation, RSV-experienced AGMs do not have detectable D25-competing antibody titers prior to immunization. However, all three mRNA antigens induced high competing antibodies titers, demonstrating that AGMs do mount an antigenic site Ø immune response after RSV infection that can be boosted by immunization. The boost in D25 competing antibody titers following mVRC-1 (v2) immunization were the highest (>450 fold), and demonstrating again similar potency at a 5-fold lower dose than the MRK-04_nopolyA_3mut. In contrast to naïve animals (cotton rats and AGMs) we observed a high boost of 4D7/post-F specific antibodies in RSV-experienced AGMs in the mVRC-1 (v2) group, demonstrating that the baseline antibody pool can determine the outcome of the epitope-specific antibody profile after immunization. Since the B cell memory pool from natural RSV infection in humans is thought to be strongly biased towards the prefusion conformation, we speculate mVRC-1 (v2) immunization in humans will boost preferentially antibodies against these epitopes, known to have more potent neutralizing activity, leading to increased efficacy over MRK-04_nopolyA_3mut.

### SEQUENCE LISTING

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a polyA tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 2)
GGGAAAUAAGAGAGAAAAGAAGAGUAAGAAGAAAUAUAAGAGCCACC
5′ UTR:
(SEQ ID NO: 77)
GGGAAAUAAGAGAGAAAAGAAGAGUAAGAAGAAAUAUAAGACCCCGG
CGCCGCCACC
3′ UTR:
(SEQ ID NO: 4)
UGAUAAUAGGCUGGAGCCUCGGUGGCCAUGCUUCUUGCCCCUUGGGC
CUCCCCCCAGCCCCUCCUCCCCUUCCUGCACCCGUACCCCCGUGGUC
UUUGAAUAAAGUCUGAGUGGGCGGC
3′ UTR:
(SEQ ID NO: 78)
UGAUAAUAGGCUGGAGCCUCGGUGGCCUAGCUUCUUGCCCCUUGGGC
CUCCCCCCAGCCCCUCCUCCCCUUCCUGCACCCGUACCCCCGUGGUC
UUUGAAUAAAGUCUGAGUGGGCGGC

TABLE 7
mVRC-1
NO: 4.

| SEQ ID NO: 1 consists of from 5′ end to 3′ end: 5′ UTR SEQ ID NO: 2, mRNA ORF SEQ ID | | 1 |
|---|---|---|
| NO: 3, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 3 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAAAACGCUGUGACC | |
| | GAGCUGCAGUUAUUGAUGCAGAGUACACCUGCCACCGGG | |
| | UCGGGCUCUGCCAUUUGCUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAAAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUGCGUUAACAAACAGGAAGGCAAAUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGCAUAACGUGAAUGCUGGGAAA | |
| | UCUACAACCAACAUCAUGAUCACUACCAUCAUUAUUGUG | |
| | AUUAUCGUAAUUCUGCUAUCCUUGAUUGCUGUCGGGCUG | |
| | CUUCUGUACUGUAAGGCCAGAUCGACGCCUGUGACCCUU | |
| | UCAAAAGACCAACUUAGCGGUAUCAAUAAUAUUGCCUUU | |
| | AGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 5 |
| acid sequence | LSALRTGWYTSVITIELSNIKENKCNGTDAKVKLIKQELDKYK | |
| | NAVTELQLLMQSTPATGSGSAICSGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGKSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

mVRC-2

| SEQ ID NO: 6 consists of from 5′ end to 3′ end: 5′ UTR SEQ ID NO: 2, mRNA ORF SEQ ID | | 6 |
|---|---|---|
| NO: 7, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 7 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAAAACGCUGUGACC | |
| | GAGCUGCAGUUAUUGAUGCAGAGUACACCUGCCACCGGG | |
| | UCGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | GGCUGCGGAGUAAGUGUGUUAACAUUUAAGGUGCUGGAC | |
| | CUCAAGAAUUAUAUUGACAAACAGUUGCUUCCUAUUCUA | |
| | AACAAACAGAGCUGUUCAAUAAGUAAUAUUGAAACUGUU | |
| | AUUGAGUUUCAGCAGAAGAACAACAGGCUUCUUGAGAUU | |
| | ACACGCGAGUUCAGUGUCAAUGCCGGCGUUACAACACCC | |
| | GUGUCUACCUACAUGCUGACGAAUUCUGAGCUUCUCUCU | |
| | CUCAUAAACGACAUGCCCAUUACGAAUGACCAAAAAAAA | |
| | CUUAUGUCCAACAACGUGCAGAUUGUGCGACAGCAAUCC | |
| | UAUAGCAUUAUGUGUAUCAUCAAGGAAGAGGUACUCGCU | |
| | UAUGUUGUGCAGCUACCACUCUAUGGUGUGAUUGACACC | |
| | CCCUGUUGGAAGCUGCAUACCAGUCCACUCUGCACCACUA | |
| | ACACAAAGGAAGGGAGCAAUAUUUGCCUCACUCGAACCG | |
| | ACAGGGGGUGGUAUUGCGAUAAUGCGGGCUCCGUGUCCU | |
| | UCUUUCCACAGGCUGAAACUUGUAAGGUACAGUCAAACC | |
| | GCGUGUUCUGUGAUACUAUGAAUUCUCGUACUCUUCCCA | |
| | GCGAGGUUAAUCUCUGCAACGUCGACAUUUUCAAUCCUA | |
| | AAUAUGACUGCAAGAUCAUGACCAGCAAGACCGACGUCU | |
| | CCAGCUCAGUAAUCACUAGCCUAGGGGCCAUUGUAAGCU | |
| | GCUAUGGCAAAACCAAGUGUACUGCCUCUAAUAAGUGCA | |
| | GAGGCAUAAUUAAAACCUUUUCAAAUGGCUGUGACUAUG | |
| | UGUCGAAUAAGGGCGUCGACACGGUCUCAGUAGGGAAUA | |
| | CCCUCUACUACGUUAACAAACAGGAAGGCAAAUCCCUUU | |
| | AUGUAAAGGGCGAGCCCAUCAUAAAUUUCUACGACCCAC | |
| | UUGUGUUCCCCAGUGAUGAAUUCGAUGCAUCAAUCUCCC | |
| | AGGUGAACGAAAAGAUCAAUCAAUCCCUUGCUUUUAUAC | |
| | GAAAGUCAGAUGAACUCCUGCAUAACGUGAAUGCUGGGA | |
| | AAUCUACAACCAACAUCAUGAUCACUACCAUCAUUAUUG | |
| | UGAUUAUCGUAAUUCUGCUAUCCUUGAUUGCUGUCGGGC | |
| | UGCUUCUGUACUGUAAGGCCAGAUCGACGCCUGUGACCC | |
| | UUUCAAAAGACCAACUUAGCGGUAUCAAUAAUAUUGCCU | |
| | UUAGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 8 |
| acid sequence | LSALRTGWYTSVITIELSNIKENKCNGTDAKVKLIKQELDKYK | |
| | NAVTELQLLMQSTPATGSGSAIASGVAVCKVLHLEGEVNKIK | |
| | SALLSTNKAVVSLSGCGVSVLTFKVLDLKNYIDKQLLPILNKQ | |
| | SCSISNIETVIEFQQKNNRLLEITREFSVNAGVTTPVSTYMLTNS | |
| | ELLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | CRGIIKTFSNGCDYVSNKGVDTVSVGNTLYYVNKQEGKSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

mVRC-3

| SEQ ID NO: 9 consists of from 5′ end to 3′ end: 5′ UTR SEQ ID NO: 2, mRNA ORF SEQ ID | | 9 |
|---|---|---|
| NO: 10, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 10 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAAAACGCUGUGACC | |
| | GAGCUGCAGUUAUUGAUGCAGAGUACACCUGCCACCAAU | |
| | AACGGGUCGGGCUCUGCCAUUGCUUCCGGCGUGGCUGUA | |
| | UGUAAAGUUCUCCACCUCGAGGGAGAGGUUAAUAAGAUU | |
| | AAGUCGGCCCUGCUGAGUACUAACAAAGCAGUGGUGUCG | |
| | CUGAGUAACGGAGUAAGUGUGUUAACAUUUAAGGUGCUG | |
| | GACCUCAAGAAUUAUAUUGACAAACAGUUGCUUCCUAUU | |
| | CUAAACAAACAGAGCUGUUCAAUAAGUAAUAUUGAAACU | |
| | GUUAUUGAGUUUCAGCAGAAGAACAACAGGCUUCUUGAG | |
| | AUUACACGCGAGUUCAGUGUCAAUGCCGGCGUUACAACA | |
| | CCCGUGUCUACCUACAUGCUGACGAAUUCUGAGCUUCUC | |
| | UCUCUCAUAAACGACAUGCCCAUUACGAAUGACCAAAAA | |
| | AAACUUAUGUCCAACAACGUGCAGAUUGUGCGACAGCAA | |
| | UCCUAUAGCAUUAUGUGUAUCAUCAAGGAAGAGGUACUC | |
| | GCUUAUGUUGUGCAGCUACCACUCUAUGGUGUGAUUGAC | |
| | ACCCCCUGUUGGAAGCUGCAUACCAGUCCACUCUGCACCA | |
| | CUAACACAAAGGAAGGGAGCAAUAUUUGCCUCACUCGAA | |
| | CCGACAGGGGGUGGUAUUGCGAUAAUGCGGGCUCCGUGU | |
| | CCUUCUUUCCACAGGCUGAAACUUGUAAGGUACAGUCAA | |
| | ACCGCGUGUUCUGUGAUACUAUGAAUUCUCGUACUCUUC | |
| | CCAGCGAGGUUAAUCUCUGCAACGUCGACAUUUUCAAUC | |
| | CUAAAUAUGACUGCAAGAUCAUGACCAGCAAGACCGACG | |
| | UCUCCAGCUCAGUAAUCACUAGCCUAGGGGCCAUUGUAA | |
| | GCUGCUAUGGCAAAACCAAGUGUACUGCCUCUAAUAAGA | |
| | ACAGAGGCAUAAUUAAAACCUUUUCAAAUGGCUGUGACU | |
| | AUGUGUCGAAUAAGGGCGUCGACACGGUCUCAGUAGGGA | |
| | AUACCCUCUACUACGUUAACAAACAGGAAGGCAAAUCCC | |
| | UUUAUGUAAAGGGCGAGCCCAUCAUAAAUUUCUACGACC | |
| | CACUUGUGUUCCCCAGUGAUGAAUUCGAUGCAUCAAUCU | |
| | CCCAGGUGAACGAAAAGAUCAAUCAAUCCCUUGCUUUUA | |
| | UACGAAAGUCAGAUGAACUCCUGCAUAACGUGAAUGCUG | |
| | GGAAAUCUACAACCAACAUCAUGAUCACUACCAUCAUUA | |
| | UUGUGAUUAUCGUAAUUCUGCUAUCCUUGAUUGCUGUCG | |
| | GGCUGCUUCUGUACUGUAAGGCCAGAUCGACGCCUGUGA | |
| | CCCUUUCAAAAGACCAACUUAGCGGUAUCAAUAAUAUUG | |
| | CCUUUAGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 11 |
| acid sequence | LSALRTGWYTSVITIELSNIKENKCNGTDAKVKLIKQELDKYK | |
| | NAVTELQLLMQSTPATNNGSGSAIASGVAVCKVLHLEGEVNK | |
| | IKSALLSTNKAVVSLSNGVSVLTFKVLDLKNYIDKQLLPILNK | |
| | QSCSISNIETVIEFQQKNNRLLEITREFSVNAGVTTPVSTYMLT | |
| | NSELLSLINDMPITNDQKKLMSNNVQIVRQQSYSIMCIIKEEVL | |
| | AYVVQLPLYGVIDTPCWKLHTSPLCTTNTKEGSNICLTRTDRG | |
| | WYCDNAGSVSFFPQAETCKVQSNRVFCDTMNSRTLPSEVNLC | |
| | NVDIFNPKYDCKIMTSKTDVSSSVITSLGAIVSCYGKTKCTASN | |
| | KNRGIIKTFSNGCDYVSNKGVDTVSVGNTLYYVNKQEGKSLY | |
| | VKGEPIINFYDPLVFPSDEFDASISQVNEKINQSLAFIRKSDELL | |
| | HNVNAGKSTTNIMITTIIIVIIVILLSLIAVGLLLYCKARSTPVTL | |
| | SKDQLSGINNIAFSN | |
| PolyA tail | 100 nt | |

VRC-1

| SEQ ID NO: 12 consists of from 5′ end to 3′ end: 5′ UTR SEQ ID NO: 2, mRNA ORF SEQ ID | | 12 |
|---|---|---|
| NO: 13, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 13 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAAAACGCUGUGACC | |
| | GAGCUGCAGUUAUUGAUGCAGAGUACACCUGCCACCGGG | |
| | UCGGGCUCUGCCAUUUGCUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAAAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUGCGUUAACAAACAGGAAGGCAAAUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGUCCGCCAUCGGUGGCUAUAUC | |
| | CCAGAAGCCCCAAGAGACGGACAAGCGUACGUCCGGAAA | |
| | GAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 14 |
| acid sequence | LSALRTGWYTSVITIELSNIKENKCNGTDAKVKLIKQELDKYK | |
| | NAVTELQLLMQSTPATGSGSAICSGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGKSLYV | |
| | KGEPIINFYDPLVFPSDEFDASISQVNEKINQSLAFIRKSDELLS | |
| | AIGGYIPEAPRDGQAYVRKDGEWVLLSTFL | |
| PolyA tail | 100 nt | |

VRC-2

| SEQ ID NO: 15 consists of from 5′ end to 3′ end: 5′ UTR SEQ ID NO: 2, mRNA ORF SEQ ID | | 15 |
|---|---|---|
| NO: 16, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 16 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAAAACGCUGUGACC | |
| | GAGCUGCAGUUAUUGAUGCAGAGUACACCUGCCACCGGG | |
| | UCGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | GGCUGCGGAGUAAGUGUGUUAACAUUUAAGGUGCUGGAC | |
| | CUCAAGAAUUAUAUUGACAAACAGUUGCUUCCUAUUCUA | |
| | AACAAACAGAGCUGUUCAAUAAGUAAUAUUGAAACUGUU | |
| | AUUGAGUUUCAGCAGAAGAACAACAGGCUUCUUGAGAUU | |
| | ACACGCGAGUUCAGUGUCAAUGCCGGCGUUACAACACCC | |
| | GUGUCUACCUACAUGCUGACGAAUUCUGAGCUUCUCUCU | |
| | CUCAUAAACGACAUGCCCAUUACGAAUGACCAAAAAAAA | |
| | CUUAUGUCCAACAACGUGCAGAUUGUGCGACAGCAAUCC | |
| | UAUAGCAUUAUGUGUAUCAUCAAGGAAGAGGUACUCGCU | |
| | UAUGUUGUGCAGCUACCACUCUAUGGUGUGAUUGACACC | |
| | CCCUGUUGGAAGCUGCAUACCAGUCCACUCUGCACCACUA | |
| | ACACAAAGGAAGGGAGCAAUAUUUGCCUCACUCGAACCG | |
| | ACAGGGGGUGGUAUUGCGAUAAUGCGGGCUCCGUGUCCU | |
| | UCUUUCCACAGGCUGAAACUUGUAAGGUACAGUCAAACC | |
| | GCGUGUUCUGUGAUACUAUGAAUUCUCGUACUCUUCCCA | |
| | GCGAGGUUAAUCUCUGCAACGUCGACAUUUUCAAUCCUA | |
| | AAUAUGACUGCAAGAUCAUGACCAGCAAGACCGACGUCU | |
| | CCAGCUCAGUAAUCACUAGCCUAGGGGCCAUUGUAAGCU | |
| | GCUAUGGCAAAACCAAGUGUACUGCCUCUAAUAAGUGCA | |
| | GAGGCAUAAUUAAAACCUUUUCAAAUGGCUGUGACUAUG | |
| | UGUCGAAUAAGGGCGUCGACACGGUCUCAGUAGGGAAUA | |
| | CCCUCUACUACGUUAACAAACAGGAAGGCAAAUCCCUUU | |
| | AUGUAAAGGGCGAGCCCAUCAUAAAUUUCUACGACCCAC | |
| | UUGUGUUCCCCAGUGAUGAAUUCGAUGCAUCAAUCUCCC | |
| | AGGUGAACGAAAAGAUCAAUCAAUCCCUUGCUUUUAUAC | |
| | GAAAGUCAGAUGAACUCCUGUCCGCCAUCGGUGGCUAUA | |
| | UCCCAGAAGCCCCAAGAGACGGACAAGCGUACGUCCGGA | |
| | AAGAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 17 |
| acid sequence | LSALRTGWYTSVITIELSNIKENKCNGTDAKVKLIKQELDKYK | |
| | NAVTELQLLMQSTPATGSGSAIASGVAVCKVLHLEGEVNKIK | |
| | SALLSTNKAVVSLSGCGVSVLTFKVLDLKNYIDKQLLPILNKQ | |
| | SCSISNIETVIEFQQKNNRLLEITREFSVNAGVTTPVSTYMLTNS | |
| | ELLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | CRGIIKTFSNGCDYVSNKGVDTVSVGNTLYYVNKQEGKSLYV | |
| | KGEPIINFYDPLVFPSDEFDASISQVNEKINQSLAFIRKSDELLS | |
| | AIGGYIPEAPRDGQAYVRKDGEWVLLSTFL | |
| PolyA tail | 100 nt | |

VRC-3

| SEQ ID NO: 18 consists of from 5′ end to 3′ end: 5′ UTR SEQ ID NO: 2, mRNA ORF SEQ ID | | 18 |
|---|---|---|
| NO: 19, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 19 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAAAACGCUGUGACC | |
| | GAGCUGCAGUUAUUGAUGCAGAGUACACCUGCCACCAAU | |
| | AACGGGUCGGGCUCUGCCAUUGCUUCCGGCGUGGCUGUA | |
| | UGUAAAGUUCUCCACCUCGAGGGAGAGGUUAAUAAGAUU | |
| | AAGUCGGCCCUGCUGAGUACUAACAAAGCAGUGGUGUCG | |
| | CUGAGUAACGGAGUAAGUGUGUUAACAUUUAAGGUGCUG | |
| | GACCUCAAGAAUUAUAUUGACAAACAGUUGCUUCCUAUU | |
| | CUAAACAAACAGAGCUGUUCAAUAAGUAAUAUUGAAACU | |
| | GUUAUUGAGUUUCAGCAGAAGAACAACAGGCUUCUUGAG | |
| | AUUACACGCGAGUUCAGUGUCAAUGCCGGCGUUACAACA | |
| | CCCGUGUCUACCUACAUGCUGACGAAUUCUGAGCUUCUC | |
| | UCUCUCAUAAACGACAUGCCCAUUACGAAUGACCAAAAA | |
| | AAACUUAUGUCCAACAACGUGCAGAUUGUGCGACAGCAA | |
| | UCCUAUAGCAUUAUGUGUAUCAUCAAGGAAGAGGUACUC | |
| | GCUUAUGUUGUGCAGCUACCACUCUAUGGUGUGAUUGAC | |
| | ACCCCCUGUUGGAAGCUGCAUACCAGUCCACUCUGCACCA | |
| | CUAACACAAAGGAAGGGAGCAAUAUUUGCCUCACUCGAA | |
| | CCGACAGGGGGUGGUAUUGCGAUAAUGCGGGCUCCGUGU | |
| | CCUUCUUUCCACAGGCUGAAACUUGUAAGGUACAGUCAA | |
| | ACCGCGUGUUCUGUGAUACUAUGAAUUCUCGUACUCUUC | |
| | CCAGCGAGGUUAAUCUCUGCAACGUCGACAUUUUCAAUC | |
| | CUAAAUAUGACUGCAAGAUCAUGACCAGCAAGACCGACG | |
| | UCUCCAGCUCAGUAAUCACUAGCCUAGGGGCCAUUGUAA | |
| | GCUGCUAUGGCAAAACCAAGUGUACUGCCUCUAAUAAGA | |
| | ACAGAGGCAUAAUUAAAACCUUUUCAAAUGGCUGUGACU | |
| | AUGUGUCGAAUAAGGGCGUCGACACGGUCUCAGUAGGGA | |
| | AUACCCUCUACUACGUUAACAAACAGGAAGGCAAAUCCC | |
| | UUUAUGUAAAGGGCGAGCCCAUCAUAAAUUUCUACGACC | |
| | CACUUGUGUUCCCCAGUGAUGAAUUCGAUGCAUCAAUCU | |
| | CCCAGGUGAACGAAAAGAUCAAUCAAUCCCUUGCUUUUA | |
| | UACGAAAGUCAGAUGAACUCCUGUCCGCCAUCGGUGGCU | |
| | AUAUCCCAGAAGCCCCAAGAGACGGACAAGCGUACGUCC | |
| | GGAAAGAUGGUGAGUGGGUCCUCCUCUCUACCUUUCUU | |
| 3′ UTR | UGAUAAUAGGCUGGAGCCUCGGUGGCCAUGCUUCUUGCC | |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 20 |
| acid sequence | LSALRTGWYTSVITIELSNIKENKCNGTDAKVKLIKQELDKYK | |
| | NAVTELQLLMQSTPATNNGSGSAIASGVAVCKVLHLEGEVNK | |
| | IKSALLSTNKAVVSLSNGVSVLTFKVLDLKNYIDKQLLPILNK | |
| | QSCSISNIETVIEFQQKNNRLLEITREFSVNAGVTTPVSTYMLT | |
| | NSELLSLINDMPITNDQKKLMSNNVQIVRQQSYSIMCIIKEEVL | |
| | AYVVQLPLYGVIDTPCWKLHTSPLCTTNTKEGSNICLTRTDRG | |
| | WYCDNAGSVSFFPQAETCKVQSNRVFCDTMNSRTLPSEVNLC | |
| | NVDIFNPKYDCKIMTSKTDVSSSVITSLGAIVSCYGKTKCTASN | |
| | KNRGIIKTFSNGCDYVSNKGVDTVSVGNTLYYVNKQEGKSLY | |
| | VKGEPIINFYDPLVFPSDEFDASISQVNEKINQSLAFIRKSDELL | |
| | SAIGGYIPEAPRDGQAYVRKDGEWVLLSTFL | |
| PolyA tail | 100 nt | |

mVRC-1 (v2)

| SEQ ID NO: 21 consists of from 5′ end to 3′ end: 5′ UTR SEQ ID NO: 2, mRNA ORF SEQ ID | | 21 |
|---|---|---|
| NO: 22, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 22 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCGGG | |
| | UCGGGCUCUGCCAUUUGCUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUGCGUUAACAAACAGGAAGGCAAAUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGCAUAACGUGAAUGCUGGGAAA | |
| | UCUACAACCAACAUCAUGAUCACUACCAUCAUUAUUGUG | |
| | AUUAUCGUAAUUCUGCUAUCCUUGAUUGCUGUCGGGCUG | |
| | CUUCUGUACUGUAAGGCCAGAUCGACGCCUGUGACCCUU | |
| | UCAAAAGACCAACUUAGCGGUAUCAAUAAUAUUGCCUUU | |
| | AGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 5 |
| acid sequence | LSALRTGWYTSVITIELSNIKENKCNGTDAKVKLIKQELDKYK | |
| | NAVTELQLLMQSTPATGSGSAICSGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGKSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

mVRC-2 (v2)

| SEQ ID NO: 23 consists of from 5′ end to 3′ end: 5′ UTR SEQ ID NO: 2, mRNA ORF SEQ ID | | 23 |
|---|---|---|
| NO: 24, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 24 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCGGG | |
| | UCGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | GGCUGCGGAGUAAGUGUGUUAACAUUUAAGGUGCUGGAC | |
| | CUCAAGAAUUAUAUUGACAAACAGUUGCUUCCUAUUCUA | |
| | AACAAACAGAGCUGUUCAAUAAGUAAUAUUGAAACUGUU | |
| | AUUGAGUUUCAGCAGAAGAACAACAGGCUUCUUGAGAUU | |
| | ACACGCGAGUUCAGUGUCAAUGCCGGCGUUACAACACCC | |
| | GUGUCUACCUACAUGCUGACGAAUUCUGAGCUUCUCUCU | |
| | CUCAUAAACGACAUGCCCAUUACGAAUGACCAAAAGAAA | |
| | CUUAUGUCCAACAACGUGCAGAUUGUGCGACAGCAAUCC | |
| | UAUAGCAUUAUGUGUAUCAUCAAGGAAGAGGUACUCGCU | |
| | UAUGUUGUGCAGCUACCACUCUAUGGUGUGAUUGACACC | |
| | CCCUGUUGGAAGCUGCAUACCAGUCCACUCUGCACCACUA | |
| | ACACAAAGGAAGGGAGCAAUAUUUGCCUCACUCGAACCG | |
| | ACAGGGGGUGGUAUUGCGAUAAUGCGGGCUCCGUGUCCU | |
| | UCUUUCCACAGGCUGAAACUUGUAAGGUACAGUCAAACC | |
| | GCGUGUUCUGUGAUACUAUGAAUUCUCGUACUCUUCCCA | |
| | GCGAGGUUAAUCUCUGCAACGUCGACAUUUUCAAUCCUA | |
| | AAUAUGACUGCAAGAUCAUGACCAGCAAGACCGACGUCU | |
| | CCAGCUCAGUAAUCACUAGCCUAGGGGCCAUUGUAAGCU | |
| | GCUAUGGCAAAACCAAGUGUACUGCCUCUAAUAAGUGCA | |
| | GAGGCAUAAUUAAAACCUUUUCAAAUGGCUGUGACUAUG | |
| | UGUCGAAUAAGGGCGUCGACACGGUCUCAGUAGGGAAUA | |
| | CCCUCUACUACGUUAACAAACAGGAAGGCAAAUCCCUUU | |
| | AUGUAAAGGGCGAGCCCAUCAUAAAUUUCUACGACCCAC | |
| | UUGUGUUCCCCAGUGAUGAAUUCGAUGCAUCAAUCUCCC | |
| | AGGUGAACGAAAAGAUCAAUCAAUCCCUUGCUUUUAUAC | |
| | GAAAGUCAGAUGAACUCCUGCAUAACGUGAAUGCUGGGA | |
| | AAUCUACAACCAACAUCAUGAUCACUACCAUCAUUAUUG | |
| | UGAUUAUCGUAAUUCUGCUAUCCUUGAUUGCUGUCGGGC | |
| | UGCUUCUGUACUGUAAGGCCAGAUCGACGCCUGUGACCC | |
| | UUUCAAAAGACCAACUUAGCGGUAUCAAUAAUAUUGCCU | |
| | UUAGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 8 |
| acid sequence | LSALRTGWYTSVITIELSNIKENKCNGTDAKVKLIKQELDKYK | |
| | NAVTELQLLMQSTPATGSGSAIASGVAVCKVLHLEGEVNKIK | |
| | SALLSTNKAVVSLSGCGVSVLTFKVLDLKNYIDKQLLPILNKQ | |
| | SCSISNIETVIEFQQKNNRLLEITREFSVNAGVTTPVSTYMLTNS | |
| | ELLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | CRGIIKTFSNGCDYVSNKGVDTVSVGNTLYYVNKQEGKSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

mVRC-3 (v2)

| SEQ ID NO: 25 consists of from 5′ end to 3′ end: 5′ UTR SEQ ID NO: 2, mRNA ORF SEQ ID | | 25 |
|---|---|---|
| NO: 26, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 26 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCAAU | |
| | AACGGGUCGGGCUCUGCCAUUGCUUCCGGCGUGGCUGUA | |
| | UGUAAAGUUCUCCACCUCGAGGGAGAGGUUAAUAAGAUU | |
| | AAGUCGGCCCUGCUGAGUACUAACAAAGCAGUGGUGUCG | |
| | CUGAGUAACGGAGUAAGUGUGUUAACAUUUAAGGUGCUG | |
| | GACCUCAAGAAUUAUAUUGACAAACAGUUGCUUCCUAUU | |
| | CUAAACAAACAGAGCUGUUCAAUAAGUAAUAUUGAAACU | |
| | GUUAUUGAGUUUCAGCAGAAGAACAACAGGCUUCUUGAG | |
| | AUUACACGCGAGUUCAGUGUCAAUGCCGGCGUUACAACA | |
| | CCCGUGUCUACCUACAUGCUGACGAAUUCUGAGCUUCUC | |
| | UCUCUCAUAAACGACAUGCCCAUUACGAAUGACCAAAAG | |
| | AAACUUAUGUCCAACAACGUGCAGAUUGUGCGACAGCAA | |
| | UCCUAUAGCAUUAUGUGUAUCAUCAAGGAAGAGGUACUC | |
| | GCUUAUGUUGUGCAGCUACCACUCUAUGGUGUGAUUGAC | |
| | ACCCCCUGUUGGAAGCUGCAUACCAGUCCACUCUGCACCA | |
| | CUAACACAAAGGAAGGGAGCAAUAUUUGCCUCACUCGAA | |
| | CCGACAGGGGGUGGUAUUGCGAUAAUGCGGGCUCCGUGU | |
| | CCUUCUUUCCACAGGCUGAAACUUGUAAGGUACAGUCAA | |
| | ACCGCGUGUUCUGUGAUACUAUGAAUUCUCGUACUCUUC | |
| | CCAGCGAGGUUAAUCUCUGCAACGUCGACAUUUUCAAUC | |
| | CUAAAUAUGACUGCAAGAUCAUGACCAGCAAGACCGACG | |
| | UCUCCAGCUCAGUAAUCACUAGCCUAGGGGCCAUUGUAA | |
| | GCUGCUAUGGCAAAACCAAGUGUACUGCCUCUAAUAAGA | |
| | ACAGAGGCAUAAUUAAAACCUUUUCAAAUGGCUGUGACU | |
| | AUGUGUCGAAUAAGGGCGUCGACACGGUCUCAGUAGGGA | |
| | AUACCCUCUACUACGUUAACAAACAGGAAGGCAAAUCCC | |
| | UUUAUGUAAAGGGCGAGCCCAUCAUAAAUUUCUACGACC | |
| | CACUUGUGUUCCCCAGUGAUGAAUUCGAUGCAUCAAUCU | |
| | CCCAGGUGAACGAAAAGAUCAAUCAAUCCCUUGCUUUUA | |
| | UACGAAAGUCAGAUGAACUCCUGCAUAACGUGAAUGCUG | |
| | GGAAAUCUACAACCAACAUCAUGAUCACUACCAUCAUUA | |
| | UUGUGAUUAUCGUAAUUCUGCUAUCCUUGAUUGCUGUCG | |
| | GGCUGCUUCUGUACUGUAAGGCCAGAUCGACGCCUGUGA | |
| | CCCUUUCAAAAGACCAACUUAGCGGUAUCAAUAAUAUUG | |
| | CCUUUAGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 11 |
| acid sequence | LSALRTGWYTSVITIELSNIKENKCNGTDAKVKLIKQELDKYK | |
| | NAVTELQLLMQSTPATNNGSGSAIASGVAVCKVLHLEGEVNK | |
| | IKSALLSTNKAVVSLSNGVSVLTFKVLDLKNYIDKQLLPILNK | |
| | QSCSISNIETVIEFQQKNNRLLEITREFSVNAGVTTPVSTYMLT | |
| | NSELLSLINDMPITNDQKKLMSNNVQIVRQQSYSIMCIIKEEVL | |
| | AYVVQLPLYGVIDTPCWKLHTSPLCTTNTKEGSNICLTRTDRG | |
| | WYCDNAGSVSFFPQAETCKVQSNRVFCDTMNSRTLPSEVNLC | |
| | NVDIFNPKYDCKIMTSKTDVSSSVITSLGAIVSCYGKTKCTASN | |
| | KNRGIIKTFSNGCDYVSNKGVDTVSVGNTLYYVNKQEGKSLY | |
| | VKGEPIINFYDPLVFPSDEFDASISQVNEKINQSLAFIRKSDELL | |
| | HNVNAGKSTTNIMITTIIIVIIVILLSLIAVGLLLYCKARSTPVTL | |
| | SKDQLSGINNIAFSN | |
| PolyA tail | 100 nt | |

VRC-1 (v2)

| SEQ ID NO: 27 consists of from 5′ end to 3′ end: 5′ UTR SEQ ID NO: 2, mRNA ORF SEQ ID | | 27 |
|---|---|---|
| NO: 28, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 28 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCGGG | |
| | UCGGGCUCUGCCAUUUGCUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUGCGUUAACAAACAGGAAGGCAAAUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGUCCGCCAUCGGUGGCUAUAUC | |
| | CCAGAAGCCCCAAGAGACGGACAAGCGUACGUCCGGAAA | |
| | GAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 14 |
| acid sequence | LSALRTGWYTSVITIELSNIKENKCNGTDAKVKLIKQELDKYK | |
| | NAVTELQLLMQSTPATGSGSAICSGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGKSLYV | |
| | KGEPIINFYDPLVFPSDEFDASISQVNEKINQSLAFIRKSDELLS | |
| | AIGGYIPEAPRDGQAYVRKDGEWVLLSTFL | |
| PolyA tail | 100 nt | |

VRC-2 (v2)

| SEQ ID NO: 29 consists of from 5′ end to 3′ end: 5′ UTR SEQ ID NO: 2, mRNA ORF SEQ ID | | 29 |
|---|---|---|
| NO: 30, and 3′ UTR SEQ ID NO: 4. | | |
| Chemistry | 1-methylpseudouridine | |
| Cap | 7mG(5′)ppp(5′)NImpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 30 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCGGG | |
| | UCGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | GGCUGCGGAGUAAGUGUGUUAACAUUUAAGGUGCUGGAC | |
| | CUCAAGAAUUAUAUUGACAAACAGUUGCUUCCUAUUCUA | |
| | AACAAACAGAGCUGUUCAAUAAGUAAUAUUGAAACUGUU | |
| | AUUGAGUUUCAGCAGAAGAACAACAGGCUUCUUGAGAUU | |
| | ACACGCGAGUUCAGUGUCAAUGCCGGCGUUACAACACCC | |
| | GUGUCUACCUACAUGCUGACGAAUUCUGAGCUUCUCUCU | |
| | CUCAUAAACGACAUGCCCAUUACGAAUGACCAAAAGAAA | |
| | CUUAUGUCCAACAACGUGCAGAUUGUGCGACAGCAAUCC | |
| | UAUAGCAUUAUGUGUAUCAUCAAGGAAGAGGUACUCGCU | |
| | UAUGUUGUGCAGCUACCACUCUAUGGUGUGAUUGACACC | |
| | CCCUGUUGGAAGCUGCAUACCAGUCCACUCUGCACCACUA | |
| | ACACAAAGGAAGGGAGCAAUAUUUGCCUCACUCGAACCG | |
| | ACAGGGGGUGGUAUUGCGAUAAUGCGGGCUCCGUGUCCU | |
| | UCUUUCCACAGGCUGAAACUUGUAAGGUACAGUCAAACC | |
| | GCGUGUUCUGUGAUACUAUGAAUUCUCGUACUCUUCCCA | |
| | GCGAGGUUAAUCUCUGCAACGUCGACAUUUUCAAUCCUA | |
| | AAUAUGACUGCAAGAUCAUGACCAGCAAGACCGACGUCU | |
| | CCAGCUCAGUAAUCACUAGCCUAGGGGCCAUUGUAAGCU | |
| | GCUAUGGCAAAACCAAGUGUACUGCCUCUAAUAAGUGCA | |
| | GAGGCAUAAUUAAAACCUUUUCAAAUGGCUGUGACUAUG | |
| | UGUCGAAUAAGGGCGUCGACACGGUCUCAGUAGGGAAUA | |
| | CCCUCUACUACGUUAACAAACAGGAAGGCAAAUCCCUUU | |
| | AUGUAAAGGGCGAGCCCAUCAUAAAUUUCUACGACCCAC | |
| | UUGUGUUCCCCAGUGAUGAAUUCGAUGCAUCAAUCUCCC | |
| | AGGUGAACGAAAAGAUCAAUCAAUCCCUUGCUUUUAUAC | |
| | GAAAGUCAGAUGAACUCCUGUCCGCCAUCGGUGGCUAUA | |
| | UCCCAGAAGCCCCAAGAGACGGACAAGCGUACGUCCGGA | |
| | AAGAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 17 |
| acid sequence | LSALRTGWYTSVITIELSNIKENKCNGTDAKVKLIKQELDKYK | |
| | NAVTELQLLMQSTPATGSGSAIASGVAVCKVLHLEGEVNKIK | |
| | SALLSTNKAVVSLSGCGVSVLTFKVLDLKNYIDKQLLPILNKQ | |
| | SCSISNIETVIEFQQKNNRLLEITREFSVNAGVTTPVSTYMLTNS | |
| | ELLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | CRGIIKTFSNGCDYVSNKGVDTVSVGNTLYYVNKQEGKSLYV | |
| | KGEPIINFYDPLVFPSDEFDASISQVNEKINQSLAFIRKSDELLS | |
| | AIGGYIPEAPRDGQAYVRKDGEWVLLSTFL | |
| PolyA tail | 100 nt | |

VRC-3 (v2)

| SEQ ID NO: 31 consists of from 5′ end to 3′ end: 5′ UTR SEQ ID NO: 2, mRNA ORF SEQ ID | | 31 |
|---|---|---|
| NO: 32, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 32 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCAAU | |
| | AACGGGUCGGGCUCUGCCAUUGCUUCCGGCGUGGCUGUA | |
| | UGUAAAGUUCUCCACCUCGAGGGAGAGGUUAAUAAGAUU | |
| | AAGUCGGCCCUGCUGAGUACUAACAAAGCAGUGGUGUCG | |
| | CUGAGUAACGGAGUAAGUGUGUUAACAUUUAAGGUGCUG | |
| | GACCUCAAGAAUUAUAUUGACAAACAGUUGCUUCCUAUU | |
| | CUAAACAAACAGAGCUGUUCAAUAAGUAAUAUUGAAACU | |
| | GUUAUUGAGUUUCAGCAGAAGAACAACAGGCUUCUUGAG | |
| | AUUACACGCGAGUUCAGUGUCAAUGCCGGCGUUACAACA | |
| | CCCGUGUCUACCUACAUGCUGACGAAUUCUGAGCUUCUC | |
| | UCUCUCAUAAACGACAUGCCCAUUACGAAUGACCAAAAG | |
| | AAACUUAUGUCCAACAACGUGCAGAUUGUGCGACAGCAA | |
| | UCCUAUAGCAUUAUGUGUAUCAUCAAGGAAGAGGUACUC | |
| | GCUUAUGUUGUGCAGCUACCACUCUAUGGUGUGAUUGAC | |
| | ACCCCCUGUUGGAAGCUGCAUACCAGUCCACUCUGCACCA | |
| | CUAACACAAAGGAAGGGAGCAAUAUUUGCCUCACUCGAA | |
| | CCGACAGGGGGUGGUAUUGCGAUAAUGCGGGCUCCGUGU | |
| | CCUUCUUUCCACAGGCUGAAACUUGUAAGGUACAGUCAA | |
| | ACCGCGUGUUCUGUGAUACUAUGAAUUCUCGUACUCUUC | |
| | CCAGCGAGGUUAAUCUCUGCAACGUCGACAUUUUCAAUC | |
| | CUAAAUAUGACUGCAAGAUCAUGACCAGCAAGACCGACG | |
| | UCUCCAGCUCAGUAAUCACUAGCCUAGGGGCCAUUGUAA | |
| | GCUGCUAUGGCAAAACCAAGUGUACUGCCUCUAAUAAGA | |
| | ACAGAGGCAUAAUUAAAACCUUUUCAAAUGGCUGUGACU | |
| | AUGUGUCGAAUAAGGGCGUCGACACGGUCUCAGUAGGGA | |
| | AUACCCUCUACUACGUUAACAAACAGGAAGGCAAAUCCC | |
| | UUUAUGUAAAGGGCGAGCCCAUCAUAAAUUUCUACGACC | |
| | CACUUGUGUUCCCCAGUGAUGAAUUCGAUGCAUCAAUCU | |
| | CCCAGGUGAACGAAAAGAUCAAUCAAUCCCUUGCUUUUA | |
| | UACGAAAGUCAGAUGAACUCCUGUCCGCCAUCGGUGGCU | |
| | AUAUCCCAGAAGCCCCAAGAGACGGACAAGCGUACGUCC | |
| | GGAAAGAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 20 |
| acid sequence | LSALRTGWYTSVITIELSNIKENKCNGTDAKVKLIKQELDKYK | |
| | NAVTELQLLMQSTPATNNGSGSAIASGVAVCKVLHLEGEVNK | |
| | IKSALLSTNKAVVSLSNGVSVLTFKVLDLKNYIDKQLLPILNK | |
| | QSCSISNIETVIEFQQKNNRLLEITREFSVNAGVTTPVSTYMLT | |
| | NSELLSLINDMPITNDQKKLMSNNVQIVRQQSYSIMCIIKEEVL | |
| | AYVVQLPLYGVIDTPCWKLHTSPLCTTNTKEGSNICLTRTDRG | |
| | WYCDNAGSVSFFPQAETCKVQSNRVFCDTMNSRTLPSEVNLC | |
| | NVDIFNPKYDCKIMTSKTDVSSSVITSLGAIVSCYGKTKCTASN | |
| | KNRGIIKTFSNGCDYVSNKGVDTVSVGNTLYYVNKQEGKSLY | |
| | VKGEPIINFYDPLVFPSDEFDASISQVNEKINQSLAFIRKSDELL | |
| | SAIGGYIPEAPRDGQAYVRKDGEWVLLSTFL | |
| PolyA tail | 100 nt | |

mVRC-4

| SEQ ID NO: 33 consists of from 5′ end to 3′ end: 5′ UTR SEQ ID NO: 2, mRNA ORF SEQ ID | | 33 |
|---|---|---|
| NO: 34, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 34 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGGGCGCGCUCCGCACAGGAUGG | |
| | UACACCUCCGUGAUCACCAUCGAGCUCAGCAAUAUUAAA | |
| | GAGAUUAAGUGCAAUGGUACCGACGCUAAAGUCAAACUU | |
| | AUCAAGCAGGAACUCGACAAAUAUAAGAACGCUGUGACC | |
| | GACCUGCAGUUAUUGAUGCAGAGUACACCUGCCACCGGG | |
| | UCGGGCUCUGCCAUUUGCUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUACCCAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUGCGUUAACAAACAGGAAGGCCAGUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGCAUAACGUGAAUGCUGGGAAA | |
| | UCUACAACCAACAUCAUGAUCACUACCAUCAUUAUUGUG | |
| | AUUAUCGUAAUUCUGCUAUCCUUGAUUGCUGUCGGGCUG | |
| | CUUCUGUACUGUAAGGCCAGAUCGACGCCUGUGACCCUU | |
| | UCAAAAGACCAACUUAGCGGUAUCAAUAAUAUUGCCUUU | |
| | AGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 35 |
| acid sequence | LGALRTGWYTSVITIELSNIKEIKCNGTDAKVKLIKQELDKYK | |
| | NAVTDLQLLMQSTPATGSGSAICSGVAVCKVLHLEGEVNKIK | |
| | SALLSTNKAVVSLSNGVSVLTFKVLDLKNYIDKQLLPILNKQS | |
| | CSIPNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGQSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

mVRC-5

| SEQ ID NO: 36 consists of from 5′ end to 3′ end: 5′ UTR SEQ ID NO: 2, mRNA ORF SEQ ID | | 36 |
|---|---|---|
| NO: 37, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 37 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGGGCGCGCUCCGCACAGGAUGG | |
| | UACACCUCCGUGAUCACCAUCGAGCUCAGCAAUAUUAAA | |
| | GAGAUUAAGUGCAAUGGUACCGACGCUAAAGUCAAACUU | |
| | AUCAAGCAGGAACUCGACAAAUAUAAGAACGCUGUGACC | |
| | GACCUGCAGUUAUUGAUGCAGAGUACACCUGCCACCGGG | |
| | UCGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | GGCUGCGGAGUAAGUGUGUUAACAUUUAAGGUGCUGGAC | |
| | CUCAAGAAUUAUAUUGACAAACAGUUGCUUCCUAUUCUA | |
| | AACAAACAGAGCUGUUCAAUACCCAAUAUUGAAACUGUU | |
| | AUUGAGUUUCAGCAGAAGAACAACAGGCUUCUUGAGAUU | |
| | ACACGCGAGUUCAGUGUCAAUGCCGGCGUUACAACACCC | |
| | GUGUCUACCUACAUGCUGACGAAUUCUGAGCUUCUCUCU | |
| | CUCAUAAACGACAUGCCCAUUACGAAUGACCAAAAGAAA | |
| | CUUAUGUCCAACAACGUGCAGAUUGUGCGACAGCAAUCC | |
| | UAUAGCAUUAUGUGUAUCAUCAAGGAAGAGGUACUCGCU | |
| | UAUGUUGUGCAGCUACCACUCUAUGGUGUGAUUGACACC | |
| | CCCUGUUGGAAGCUGCAUACCAGUCCACUCUGCACCACUA | |
| | ACACAAAGGAAGGGAGCAAUAUUUGCCUCACUCGAACCG | |
| | ACAGGGGGUGGUAUUGCGAUAAUGCGGGCUCCGUGUCCU | |
| | UCUUUCCACAGGCUGAAACUUGUAAGGUACAGUCAAACC | |
| | GCGUGUUCUGUGAUACUAUGAAUUCUCGUACUCUUCCCA | |
| | GCGAGGUUAAUCUCUGCAACGUCGACAUUUUCAAUCCUA | |
| | AAUAUGACUGCAAGAUCAUGACCAGCAAGACCGACGUCU | |
| | CCAGCUCAGUAAUCACUAGCCUAGGGGCCAUUGUAAGCU | |
| | GCUAUGGCAAAACCAAGUGUACUGCCUCUAAUAAGUGCA | |
| | GAGGCAUAAUUAAAACCUUUUCAAAUGGCUGUGACUAUG | |
| | UGUCGAAUAAGGGCGUCGACACGGUCUCAGUAGGGAAUA | |
| | CCCUCUACUACGUUAACAAACAGGAAGGCCAGUCCCUUU | |
| | AUGUAAAGGGCGAGCCCAUCAUAAAUUUCUACGACCCAC | |
| | UUGUGUUCCCCAGUGAUGAAUUCGAUGCAUCAAUCUCCC | |
| | AGGUGAACGAAAAGAUCAAUCAAUCCCUUGCUUUUAUAC | |
| | GAAAGUCAGAUGAACUCCUGCAUAACGUGAAUGCUGGGA | |
| | AAUCUACAACCAACAUCAUGAUCACUACCAUCAUUAUUG | |
| | UGAUUAUCGUAAUUCUGCUAUCCUUGAUUGCUGUCGGGC | |
| | UGCUUCUGUACUGUAAGGCCAGAUCGACGCCUGUGACCC | |
| | UUUCAAAAGACCAACUUAGCGGUAUCAAUAAUAUUGCCU | |
| | UUAGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGGGC | |
| Corresponding amino | MELLILKANAITTILTAVTFCFASGQNITEEFYQSTCSAVSKGY | 38 |
| acid sequence | LGALRTGWYTSVITIELSNIKEIKCNGTDAKVKLIKQELDKYK | |
| | NAVTDLQLLMQSTPATGSGSAIASGVAVCKVLHLEGEVNKIK | |
| | SALLSTNKAVVSLSGCGVSVLTFKVLDLKNYIDKQLLPILNKQ | |
| | SCSIPNIETVIEFQQKNNRLLEITREFSVNAGVTTPVSTYMLTNS | |
| | ELLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | CRGIIKTFSNGCDYVSNKGVDTVSVGNTLYYVNKQEGQSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

mVRC-6

| SEQ ID NO: 39 consists of from 5′ end to 3′ end: 5′ UTR SEQ ID NO: 2, mRNA ORF SEQ ID | | 39 |
|---|---|---|
| NO: 40, and 3′ UTR SEQ ID NO: 4. | | |
| Chemistry | 1-methylpseudouridine | |
| Cap | 7mG(5′)ppp(5′)NImpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 40 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCGGG | |
| | UCGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUACGUUAACAAACAGGAAGGCAAAUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGCAUAACGUGAAUGCUGGGAAA | |
| | UCUACAACCAACAUCAUGAUCACUACCAUCAUUAUUGUG | |
| | AUUAUCGUAAUUCUGCUAUCCUUGAUUGCUGUCGGGCUG | |
| | CUUCUGUACUGUAAGGCCAGAUCGACGCCUGUGACCCUU | |
| | UCAAAAGACCAACUUAGCGGUAUCAAUAAUAUUGCCUUU | |
| | AGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 41 |
| acid sequence | LSALRTGWYTSVITIELSNIKENKCNGTDAKVKLIKQELDKYK | |
| | NAVTELQLLMQSTPATGSGSAIASGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYYVNKQEGKSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

VRC-4

| SEQ ID NO: 42 consists of from 5′ end to 3′ end: 5′ UTR SEQ ID NO: 2, mRNA ORF SEQ ID | | 42 |
|---|---|---|
| NO: 43, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 43 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGGGCGCGCUCCGCACAGGAUGG | |
| | UACACCUCCGUGAUCACCAUCGAGCUCAGCAAUAUUAAA | |
| | GAGAUUAAGUGCAAUGGUACCGACGCUAAAGUCAAACUU | |
| | AUCAAGCAGGAACUCGACAAAUAUAAGAACGCUGUGACC | |
| | GACCUGCAGUUAUUGAUGCAGAGUACACCUGCCACCGGG | |
| | UCGGGCUCUGCCAUUUGCUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUACCCAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUGCGUUAACAAACAGGAAGGCCAGUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGUCCGCCAUCGGUGGCUAUAUC | |
| | CCAGAAGCCCCAAGAGACGGACAAGCGUACGUCCGGAAA | |
| | GAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 44 |
| acid sequence | LGALRTGWYTSVITIELSNIKEIKCNGTDAKVKLIKQELDKYK | |
| | NAVTDLQLLMQSTPATGSGSAICSGVAVCKVLHLEGEVNKIK | |
| | SALLSTNKAVVSLSNGVSVLTFKVLDLKNYIDKQLLPILNKQS | |
| | CSIPNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGQSLYV | |
| | KGEPIINFYDPLVFPSDEFDASISQVNEKINQSLAFIRKSDELLS | |
| | AIGGYIPEAPRDGQAYVRKDGEWVLLSTFL | |
| PolyA tail | 100 nt | |

VRC-5

| SEQ ID NO: 45 consists of from 5′ end to 3′ end: 5′ UTR SEQ ID NO: 2, mRNA ORF SEQ ID | | 45 |
|---|---|---|
| NO: 46, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 46 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGGGCGCGCUCCGCACAGGAUGG | |
| | UACACCUCCGUGAUCACCAUCGAGCUCAGCAAUAUUAAA | |
| | GAGAUUAAGUGCAAUGGUACCGACGCUAAAGUCAAACUU | |
| | AUCAAGCAGGAACUCGACAAAUAUAAGAACGCUGUGACC | |
| | GACCUGCAGUUAUUGAUGCAGAGUACACCUGCCACCGGG | |
| | UCGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | GGCUGCGGAGUAAGUGUGUUAACAUUUAAGGUGCUGGAC | |
| | CUCAAGAAUUAUAUUGACAAACAGUUGCUUCCUAUUCUA | |
| | AACAAACAGAGCUGUUCAAUACCCAAUAUUGAAACUGUU | |
| | AUUGAGUUUCAGCAGAAGAACAACAGGCUUCUUGAGAUU | |
| | ACACGCGAGUUCAGUGUCAAUGCCGGCGUUACAACACCC | |
| | GUGUCUACCUACAUGCUGACGAAUUCUGAGCUUCUCUCU | |
| | CUCAUAAACGACAUGCCCAUUACGAAUGACCAAAAGAAA | |
| | CUUAUGUCCAACAACGUGCAGAUUGUGCGACAGCAAUCC | |
| | UAUAGCAUUAUGUGUAUCAUCAAGGAAGAGGUACUCGCU | |
| | UAUGUUGUGCAGCUACCACUCUAUGGUGUGAUUGACACC | |
| | CCCUGUUGGAAGCUGCAUACCAGUCCACUCUGCACCACUA | |
| | ACACAAAGGAAGGGAGCAAUAUUUGCCUCACUCGAACCG | |
| | ACAGGGGGUGGUAUUGCGAUAAUGCGGGCUCCGUGUCCU | |
| | UCUUUCCACAGGCUGAAACUUGUAAGGUACAGUCAAACC | |
| | GCGUGUUCUGUGAUACUAUGAAUUCUCGUACUCUUCCCA | |
| | GCGAGGUUAAUCUCUGCAACGUCGACAUUUUCAAUCCUA | |
| | AAUAUGACUGCAAGAUCAUGACCAGCAAGACCGACGUCU | |
| | CCAGCUCAGUAAUCACUAGCCUAGGGGCCAUUGUAAGCU | |
| | GCUAUGGCAAAACCAAGUGUACUGCCUCUAAUAAGUGCA | |
| | GAGGCAUAAUUAAAACCUUUUCAAAUGGCUGUGACUAUG | |
| | UGUCGAAUAAGGGCGUCGACACGGUCUCAGUAGGGAAUA | |
| | CCCUCUACUACGUUAACAAACAGGAAGGCCAGUCCCUUU | |
| | AUGUAAAGGGCGAGCCCAUCAUAAAUUUCUACGACCCAC | |
| | UUGUGUUCCCCAGUGAUGAAUUCGAUGCAUCAAUCUCCC | |
| | AGGUGAACGAAAAGAUCAAUCAAUCCCUUGCUUUUAUAC | |
| | GAAAGUCAGAUGAACUCCUGUCCGCCAUCGGUGGCUAUA | |
| | UCCCAGAAGCCCCAAGAGACGGACAAGCGUACGUCCGGA | |
| | AAGAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 47 |
| acid sequence | LGALRTGWYTSVITIELSNIKEIKCNGTDAKVKLIKQELDKYK | |
| | NAVTDLQLLMQSTPATGSGSAIASGVAVCKVLHLEGEVNKIK | |
| | SALLSTNKAVVSLSGCGVSVLTFKVLDLKNYIDKQLLPILNKQ | |
| | SCSIPNIETVIEFQQKNNRLLEITREFSVNAGVTTPVSTYMLTNS | |
| | ELLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | CRGIIKTFSNGCDYVSNKGVDTVSVGNTLYYVNKQEGQSLYV | |
| | KGEPIINFYDPLVFPSDEFDASISQVNEKINQSLAFIRKSDELLS | |
| | AIGGYIPEAPRDGQAYVRKDGEWVLLSTFL | |
| PolyA tail | 100 nt | |

VRC-6

| SEQ ID NO: 48 consists of from 5′ end to 3′ end: 5′ UTR SEQ ID NO: 2, mRNA ORF SEQ ID | | 48 |
|---|---|---|
| NO: 49, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 49 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCGGG | |
| | UCGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUACGUUAACAAACAGGAAGGCAAAUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGUCCGCCAUCGGUGGCUAUAUC | |
| | CCAGAAGCCCCAAGAGACGGACAAGCGUACGUCCGGAAA | |
| | GAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 50 |
| acid sequence | LSALRTGWYTSVITIELSNIKENKCNGTDAKVKLIKQELDKYK | |
| | NAVTELQLLMQSTPATGSGSAIASGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYYVNKQEGKSLYV | |
| | KGEPIINFYDPLVFPSDEFDASISQVNEKINQSLAFIRKSDELLS | |
| | AIGGYIPEAPRDGQAYVRKDGEWVLLSTFL | |
| PolyA tail | 100 nt | |

RSVF mVRC1(v2) UTR v1.1

| SEQ ID NO: 51 consists of from 5′ end to 3′ end: 5′ UTR SEQ ID NO: 77, mRNA ORF SEQ | | 51 |
|---|---|---|
| ID NO: 52, and 3′ UTR SEQ ID NO: 78. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 77 |
| | AGACCCCGGCGCCGCCACC | |
| ORF of mRNA | AUGGAACUGCUCAUUUUGAAGGCAAACGCUAUCACGACA | 52 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCGGG | |
| | UCGGGCUCUGCCAUUUGCUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUGCGUUAACAAACAGGAAGGCAAAUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGCAUAACGUGAAUGCUGGGAAA | |
| | UCUACAACCAACAUCAUGAUCACUACCAUCAUUAUUGUG | |
| | AUUAUCGUAAUUCUGCUAUCCUUGAUUGCUGUCGGGCUG | |
| | CUUCUGUACUGUAAGGCCAGAUCGACGCCUGUGACCCUU | |
| | UCAAAAGACCAACUUAGCGGUAUCAAUAAUAUUGCCUUU | |
| | AGCAAU | |
| 3′ UTR | UGAUAAUAGGCUGGAGCCUCGGUGGCCUAGCUUCUUGCC | 78 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 5 |
| acid sequence | LSALRTGWYTSVITIELSNIKENKCNGTDAKVKLIKQELDKYK | |
| | NAVTELQLLMQSTPATGSGSAICSGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGKSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

MRK_RSVF mVRC1(v2)_SE_001

| SEQ ID NO: 53 consists of from 5′ end to 3′ end: 5′ UTR SEQ ID NO: 77, mRNA ORF SEQ | | 53 |
|---|---|---|
| ID NO: 54, and 3′ UTR SEQ ID NO: 78. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 77 |
| | AGACCCCGGCGCCGCCACC | |
| ORF of mRNA | AUGGAGCUGCUGAUCCUCAAGGCUAACGCCAUCACCACC | 54 |
| Construct | AUCCUGACCGCCGUGACCUUCUGCUUCGCCAGCGGGCAGA | |
| (excluding the stop | ACAUCACCGAGGAGUUCUACCAGAGCACUUGCAGCGCCG | |
| codon) | UGAGCAAGGGCUACCUGUCAGCCCUGCGGACCGGCUGGU | |
| | ACACCAGCGUGAUCACCAUCGAGCUGAGCAACAUCAAGG | |
| | AGAACAAGUGCAACGGCACCGACGCCAAGGUGAAGCUGA | |
| | UCAAGCAGGAGCUGGACAAGUACAAGAACGCCGUGACCG | |
| | AGCUGCAGCUGCUGAUGCAGUCCACGCCCGCUACCGGGUC | |
| | AGGCAGCGCCAUCUGCUCCGGCGUGGCCGUGUGCAAGGU | |
| | GCUGCACCUGGAGGGCGAGGUGAACAAGAUCAAGAGCGC | |
| | CCUGCUGAGCACCAACAAGGCCGUGGUGAGCCUGAGCAA | |
| | CGGCGUGAGCGUGCUGACCUUCAAGGUGCUGGACCUGAA | |
| | GAACUACAUCGACAAGCAGCUGCUGCCCAUCCUGAACAA | |
| | GCAGUCCUGCUCCAUCUCCAACAUCGAAACUGUGAUCGA | |
| | GUUCCAGCAGAAGAACAACCGCCUGCUGGAGAUCACCCG | |
| | CGAGUUCAGCGUGAACGCCGGCGUGACCACCCCAGUGAG | |
| | CACCUACAUGCUGACCAACAGCGAGCUGCUGAGCCUGAU | |
| | CAACGACAUGCCCAUCACCAACGACCAGAAGAAGCUGAU | |
| | GAGCAACAACGUGCAGAUCGUGCGCCAGCAGUCCUACUC | |
| | UAUCAUGUGCAUCAUCAAGGAGGAGGUGCUGGCCUACGU | |
| | GGUGCAGCUGCCCCUGUACGGCGUGAUCGACACACCCUGC | |
| | UGGAAGCUGCACACCUCCCCUCUGUGCACCACUAACACCA | |
| | AGGAGGGGAGCAACAUCUGCCUGACCCGGACCGACCGCG | |
| | GCUGGUACUGCGACAACGCCGGCAGCGUGAGCUUCUUCC | |
| | CUCAGGCCGAAACCUGCAAGGUGCAGAGCAACAGGGUGU | |
| | UCUGCGACACCAUGAACUCCCGGACCCUGCCCAGCGAGGU | |
| | GAACCUGUGCAACGUGGACAUCUUCAACCCCAAGUACGA | |
| | UUGCAAGAUCAUGACCUCCAAGACCGACGUGAGCAGCUC | |
| | CGUGAUCACCUCCCUGGGCGCCAUCGUGAGUUGCUACGG | |
| | CAAGACCAAGUGCACCGCCAGCAACAAGAACCGCGGCAUC | |
| | AUCAAGACCUUCAGCAACGGCUGCGACUACGUGUCCAAC | |
| | AAGGGCGUGGACACCGUGAGCGUGGGCAACACCCUGUAC | |
| | UGCGUGAACAAGCAGGAGGGCAAGAGCCUGUACGUGAAG | |
| | GGCGAGCCCAUCAUCAACUUCUACGAUCCGCUGGUGUUC | |
| | CCCUCCGACGAGUUCGACGCCUCCAUCAGCCAGGUGAACG | |
| | AGAAGAUCAACCAGAGCCUGGCCUUCAUCCGCAAGUCCG | |
| | ACGAGCUGCUGCACAACGUCAACGCCGGUAAGAGCACCA | |
| | CCAACAUCAUGAUCACCACAAUCAUCAUCGUGAUCAUUG | |
| | UGAUCUUGCUCUCCUUAAUCGCCGUGGGCCUGCUGCUGU | |
| | ACUGCAAGGCCCGGAGCACCCCAGUGACCCUGAGCAAAG | |
| | AUCAGCUGUCCGGCAUCAACAACAUCGCCUUCUCCAAC | |
| 3′ UTR | UGAUAAUAGGCUGGAGCCUCGGUGGCCUAGCUUCUUGCC | 78 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 5 |
| acid sequence | LSALRTGWYTSVITIELSNIKENKCNGTDAKVKLIKQELDKYK | |
| | NAVTELQLLMQSTPATGSGSAICSGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGKSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

MRK_RSVF mVRC1(v2)_SE_002

| SEQ ID NO: 55 consists of from 5′ end to 3′ end: 5′ UTR SEQ ID NO: 77, mRNA ORF SEQ | | 55 |
|---|---|---|
| ID NO: 56, and 3′ UTR SEQ ID NO: 78. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 77 |
| | AGACCCCGGCGCCGCCACC | |
| ORF of mRNA | AUGGAGCUGCUGAUUCUGAAGGCCAACGCCAUUACCACC | 56 |
| Construct | AUUCUGACCGCCGUGACCUUCUGCUUCGCCAGCGGCCAGA | |
| (excluding the stop | AUAUUACCGAGGAGUUCUACCAGUCAACCUGCAGCGCCG | |
| codon) | UGAGCAAGGGCUACCUGAGCGCCCUACGGACCGGGUGGU | |
| | ACACCUCCGUGAUCACCAUUGAGCUGAGCAAUAUUAAGG | |
| | AGAAUAAGUGCAACGGGACCGACGCCAAGGUGAAGCUCA | |
| | UCAAGCAGGAGCUGGAUAAGUACAAGAACGCCGUGACCG | |
| | AGCUGCAGCUGCUGAUGCAGAGCACACCGGCCACGGGCU | |
| | CCGGCAGCGCCAUUUGCUCAGGCGUGGCCGUGUGCAAGG | |
| | UGCUGCACCUCGAGGGCGAGGUCAAUAAGAUUAAGAGCG | |
| | CCCUGUUGAGCACCAAUAAGGCCGUGGUGAGCCUGAGCA | |
| | ACGGCGUGAGCGUGCUGACCUUCAAGGUGCUGGAUCUCA | |
| | AGAAUUACAUUGAUAAGCAGCUGCUGCCAAUUCUCAAUA | |
| | AGCAGAGCUGCAGCAUUAGCAAUAUCGAAACCGUGAUUG | |
| | AGUUCCAGCAGAAGAAUAAUCGCCUGCUGGAGAUUACCC | |
| | GCGAGUUCAGCGUCAACGCCGGGGUGACCACCCCAGUCA | |
| | GCACCUACAUGCUGACCAAUAGCGAGCUGCUGAGCCUGA | |
| | UUAACGAUAUGCCCAUUACCAACGACCAGAAGAAGCUGA | |
| | UGAGCAAUAACGUGCAGAUUGUGCGCCAGCAGAGCUACA | |
| | GCAUUAUGUGCAUUAUUAAGGAGGAGGUGCUGGCCUACG | |
| | UGGUGCAGCUGCCCCUCUACGGCGUGAUUGAUACGCCCU | |
| | GCUGGAAGCUGCACACCAGCCCACUGUGCACCACCAAUAC | |
| | CAAGGAGGGCAGCAAUAUUUGCCUGACCCGCACCGAUCG | |
| | CGGGUGGUACUGCGAUAACGCCGGCAGCGUGAGCUUCUU | |
| | CCCACAGGCCGAAACAUGCAAGGUGCAGUCCAAUCGCGU | |
| | GUUCUGCGAUACCAUGAACAGCCGCACCCUGCCCAGCGAG | |
| | GUGAAUCUCUGCAACGUGGAUAUUUUCAAUCCAAAGUAC | |
| | GAUUGCAAGAUUAUGACCAGCAAGACCGACGUGAGCUCC | |
| | AGCGUGAUUACCUCACUCGGCGCCAUUGUGAGCUGCUAC | |
| | GGCAAGACCAAGUGCACCGCCUCAAAUAAGAAUCGCGGC | |
| | AUUAUUAAGACCUUCAGCAACGGCUGCGAUUACGUGAGC | |
| | AAUAAGGGCGUGGAUACCGUGAGCGUGGGAAAUACCCUG | |
| | UACUGCGUGAAUAAGCAGGAGGGCAAGAGCCUGUACGUG | |
| | AAGGGCGAGCCAAUUAUUAAUUUCUACGAUCCCCUGGUG | |
| | UUCCCCAGCGACGAGUUCGACGCCAGCAUUAGCCAGGUG | |
| | AACGAGAAGAUUAAUCAGAGCCUGGCCUUCAUUCGCAAG | |
| | UCCGACGAGCUCCUGCACAACGUGAACGCCGGCAAGAGC | |
| | ACCACCAACAUCAUGAUUACCACCAUCAUCAUUGUGAUU | |
| | AUCGUGAUCCUCCUCAGCCUCAUUGCCGUGGGCCUCCUCC | |
| | UGUACUGCAAGGCCCGCAGCACUCCUGUGACCCUCAGCAA | |
| | GGAUCAGCUGAGCGGCAUCAAUAAUAUCGCCUUCAGCAAU | |
| 3′ UTR | UGAUAAUAGGCUGGAGCCUCGGUGGCCUAGCUUCUUGCC | 78 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 5 |
| acid sequence | LSALRTGWYTSVITIELSNIKENKCNGTDAKVKLIKQELDKYK | |
| | NAVTELQLLMQSTPATGSGSAICSGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGKSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

MRK_RSVF mVRC1(v2)_SE_003

| SEQ ID NO: 57 consists of from 5′ end to 3′ end: 5′ UTR SEQ ID NO: 77, mRNA ORF SEQ | | 57 |
|---|---|---|
| ID NO: 58, and 3′ UTR SEQ ID NO: 78. | | |
| Chemistry | 1-methylpseudouridine | |
| Cap | 7mG(5′)ppp(5′)NImpNp | |
| 5′ UTR | GGGAAAUAAGAGAGAAAAGAAGAGUAAGAAGAAAUAUA | 77 |
| | AGACCCCGGCGCCGCCACC | |
| ORF of mRNA | AUGGAGCUGCUGAUCCUGAAGGCCAACGCCAUCACCACC | 58 |
| Construct | AUCCUGACCGCCGUGACCUUCUGCUUCGCCAGCGGCCAGA | |
| (excluding the stop | ACAUUACCGAGGAGUUCUACCAGUCCACCUGCAGCGCCG | |
| codon) | UGAGCAAGGGCUACCUGAGCGCCCUGCGCACCGGGUGGU | |
| | ACACCAGCGUGAUCACCAUCGAGCUGUCCAACAUCAAGG | |
| | AGAACAAGUGCAACGGCACAGACGCCAAGGUGAAGCUGA | |
| | UCAAGCAGGAGCUGGACAAGUACAAGAACGCCGUGACCG | |
| | AGCUCCAGCUGCUGAUGCAGUCCACCCCGGCCACCGGCAG | |
| | UGGAAGUGCUAUCUGCAGCGGGGUGGCCGUGUGCAAGGU | |
| | GCUGCACCUGGAGGGCGAGGUGAACAAGAUCAAGAGCGC | |
| | CCUGCUGAGCACCAACAAGGCCGUGGUGAGCCUGAGCAA | |
| | CGGGGUGUCCGUGCUGACAUUCAAGGUGCUGGACCUGAA | |
| | GAACUACAUCGACAAGCAGCUGCUGCCCAUCCUCAACAA | |
| | GCAGAGCUGCUCCAUCUCCAAUAUCGAAACCGUGAUCGA | |
| | GUUUCAGCAGAAGAACAACCGGCUGCUGGAGAUCACCCG | |
| | GGAGUUCUCCGUGAACGCCGGGGUGACCACCCCAGUGAG | |
| | CACCUACAUGCUGACCAACUCCGAGCUGCUGAGCCUGAUC | |
| | AACGAUAUGCCAAUCACAAACGAUCAGAAGAAGCUGAUG | |
| | AGCAACAACGUGCAGAUCGUGCGGCAGCAGUCCUACAGC | |
| | AUCAUGUGCAUCAUCAAGGAGGAGGUGCUGGCCUACGUG | |
| | GUGCAGCUGCCCCUGUACGGCGUGAUCGACACUCCUUGC | |
| | UGGAAGCUGCACACCUCACCGCUGUGCACCACGAACACUA | |
| | AGGAGGGCAGCAACAUCUGCCUGACCCGGACCGACCGAG | |
| | GCUGGUACUGCGACAACGCCGGCAGCGUCUCUUUCUUUC | |
| | CGCAGGCCGAGACGUGCAAGGUGCAGUCCAACCGGGUGU | |
| | UCUGCGACACCAUGAACUCCCGGACCCUGCCCUCCGAGGU | |
| | GAACCUGUGCAACGUGGACAUUUUCAACCCCAAGUACGA | |
| | CUGCAAGAUCAUGACCAGUAAGACCGACGUGUCCAGCUC | |
| | CGUAAUCACCAGCCUGGGCGCCAUCGUGUCCUGUUACGG | |
| | CAAGACCAAGUGUACCGCCUCCAACAAGAACCGAGGUAU | |
| | CAUCAAGACCUUCUCCAACGGGUGCGACUACGUGUCCAA | |
| | CAAGGGCGUGGACACCGUGAGCGUGGGAAAUACCCUCUA | |
| | CUGCGUGAACAAGCAGGAGGGGAAGUCCCUGUACGUGAA | |
| | GGGUGAGCCCAUCAUCAACUUUUACGAUCCCCUGGUGUU | |
| | CCCCAGCGACGAGUUCGACGCAUCCAUUAGCCAGGUGAA | |
| | CGAGAAGAUCAACCAGAGUCUGGCCUUCAUCCGCAAGUC | |
| | CGACGAGCUGCUGCACAACGUGAACGCUGGCAAGUCAAC | |
| | AACCAACAUCAUGAUCACGACCAUCAUUAUCGUGAUCAU | |
| | CGUGAUCCUGCUGUCCCUGAUCGCAGUGGGCCUGCUCCU | |
| | GUACUGCAAGGCCCGCAGCACACCCGUGACCCUCAGCAAG | |
| | GAUCAGCUCAGCGGCAUCAACAACAUCGCCUUCAGCAAC | |
| 3′ UTR | UGAUAAUAGGCUGGAGCCUCGGUGGCCUAGCUUCUUGCC | 78 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 5 |
| acid sequence | LSALRTGWYTSVITIELSNIKENKCNGTDAKVKLIKQELDKYK | |
| | NAVTELQLLMQSTPATGSGSAICSGVAVCKVLHLEGEVNKIK | |
| | SALLSTNKAVVSLSNGVSVLTFKVLDLKNYIDKQLLPILNKQS | |
| | CSISNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGKSLYV | |
| | KGEPIINFYDPLVFPSDEFDASISQVNEKINQSLAFIRKSDELLH | |
| | NVNAGKSTTNIMITTIIIVIIVILLSLIAVGLLLYCKARSTPVTLS | |
| | KDQLSGINNIAFSN | |
| PolyA tail | 100 nt | |

MRK_SLZF129

| SEQ ID NO: 59 consists of from 5′ end to 3′ end: 5′ UTR SEQ ID NO: 2, mRNA ORF SEQ ID | | 59 |
|---|---|---|
| NO: 60, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 60 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGGGAGGCGGUAGCGGUGGCGGA | |
| | UCCGGCGGUGGAAGCGGCUCUGCCAUUGCUUCCGGCGUG | |
| | GCUGUAUGUAAAGUUCUCCACCUCGAGGGAGAGGUUAAU | |
| | AAGAUUAAGUCGGCCCUGCUGAGUACUAACAAAGCAGUG | |
| | GUGUCGCUGAGUAACGGAGUAAGUGUGUUAACAUUUAAG | |
| | GUGCUGGACCUCAAGAAUUAUAUUGACAAACAGUUGCUU | |
| | CCUAUUCUAAACAAACAGAGCUGUUCAAUAAGUAAUAUU | |
| | GAAACUGUUAUUGAGUUUCAGCAGAAGAACAACAGGCUU | |
| | CUUGAGAUUACACGCGAGUUCAGUGUCAAUGCCGGCGUU | |
| | ACAACACCCGUGUCUACCUACAUGCUGACGAAUUCUGAG | |
| | CUUCUCUCUCUCAUAAACGACAUGCCCAUUACGAAUGAC | |
| | CAAAAGAAACUUAUGUCCAACAACGUGCAGAUUGUGCGA | |
| | CAGCAAUCCUAUAGCAUUAUGUGUAUCAUCAAGGAAGAG | |
| | GUACUCGCUUAUGUUGUGCAGCUACCACUCUAUGGUGUG | |
| | AUUGACACCCCCUGUUGGAAGCUGCAUACCAGUCCACUC | |
| | UGCACCACUAACACAAAGGAAGGGAGCAAUAUUUGCCUC | |
| | ACUCGAACCGACAGGGGGUGGUAUUGCGAUAAUGCGGGC | |
| | UCCGUGUCCUUCUUUCCACAGGCUGAAACUUGUAAGGUA | |
| | CAGUCAAACCGCGUGUUCUGUGAUACUAUGAAUUCUCUG | |
| | ACUCUUCCCAGCGAGGUUAAUCUCUGCAACGUCGACAUU | |
| | UUCAAUCCUAAAUAUGACUGCAAGAUCAUGACCAGCAAG | |
| | ACCGACGUCUCCAGCUCAGUAAUCACUAGCCUAGGGGCC | |
| | AUUGUAAGCUGCUAUGGCAAAACCAAGUGUACUGCCUCU | |
| | AAUAAGAACAGAGGCAUAAUUAAAACCUUUUCAAAUGGC | |
| | UGUGACUAUGUGUCGAAUAAGGGCGUCGACACGGUCUCA | |
| | GUAGGGAAUACCCUCUACUACGUUAACAAACAGGAAGGC | |
| | AAAUCCCUUUAUGUAAAGGGCGAGCCCAUCAUAAAUUUC | |
| | UACGACCCACUUUCCGCCAUCGGUGGCUAUAUCCCAGAA | |
| | GCCCCAAGAGACGGACAAGCGUACGUCCGGAAAGAUGGU | |
| | GAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 61 |
| acid sequence | LSALRTGWYTSVITIELSNIKENKCNGTDAKVKLIKQELDKYK | |
| | NAVTELQLLMGGGSGGGSGGGSGSAIASGVAVCKVLHLEGE | |
| | VNKIKSALLSTNKAVVSLSNGVSVLTFKVLDLKNYIDKQLLPI | |
| | LNKQSCSISNIETVIEFQQKNNRLLEITREFSVNAGVTTPVSTY | |
| | MLTNSELLSLINDMPITNDQKKLMSNNVQIVRQQSYSIMCIIKE | |
| | EVLAYVVQLPLYGVIDTPCWKLHTSPLCTTNTKEGSNICLTRT | |
| | DRGWYCDNAGSVSFFPQAETCKVQSNRVFCDTMNSLTLPSEV | |
| | NLCNVDIFNPKYDCKIMTSKTDVSSSVITSLGAIVSCYGKTKCT | |
| | ASNKNRGIIKTFSNGCDYVSNKGVDTVSVGNTLYYVNKQEGK | |
| | SLYVKGEPIINFYDPLSAIGGYIPEAPRDGQAYVRKDGEWVLL | |
| | STFL | |
| PolyA tail | 100 nt | |

MRK_SLZF128

| SEQ ID NO: 62 consists of from 5′ end to 3′ end: 5′ UTR SEQ ID NO: 2, mRNA ORF SEQ ID | | 62 |
|---|---|---|
| NO: 63, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 63 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCAAU | |
| | AACAGAGCUAGGAGGGAGUUGCCUAGGUUUAUGAACUAC | |
| | ACUCUCAACAACGCGAAGAAAACCAAUGUGACGCUAUCC | |
| | AAGAAACGGAAGAGGAGGUUCCUGGGGUUUCUUUUAGGG | |
| | GUGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCUGACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUACGUUAACAAACAGGAAGGCAAAUCCCUUUAU | |
| | GUAAAGGGCGAGCCCAUCAUAAAUUUCUACGACCCACUU | |
| | UCCGCCAUCGGUGGCUAUAUCCCAGAAGCCCCAAGAGAC | |
| | GGACAAGCGUACGUCCGGAAAGAUGGUGAGUGGGUCCUC | |
| | CUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 64 |
| acid sequence | LSALRTGWYTSVITIELSNIKENKCNGTDAKVKLIKQELDKYK | |
| | NAVTELQLLMQSTPATNNRARRELPRFMNYTLNNAKKTNVT | |
| | LSKKRKRRFLGFLLGVGSAIASGVAVCKVLHLEGEVNKIKSAL | |
| | LSTNKAVVSLSNGVSVLTFKVLDLKNYIDKQLLPILNKQSCSIS | |
| | NIETVIEFQQKNNRLLEITREFSVNAGVTTPVSTYMLTNSELLS | |
| | LINDMPITNDQKKLMSNNVQIVRQQSYSIMCIIKEEVLAYVVQ | |
| | LPLYGVIDTPCWKLHTSPLCTTNTKEGSNICLTRTDRGWYCD | |
| | NAGSVSFFPQAETCKVQSNRVFCDTMNSLTLPSEVNLCNVDIF | |
| | NPKYDCKIMTSKTDVSSSVITSLGAIVSCYGKTKCTASNKNRG | |
| | IIKTFSNGCDYVSNKGVDTVSVGNTLYYVNKQEGKSLYVKGE | |
| | PIINFYDPLSAIGGYIPEAPRDGQAYVRKDGEWVLLSTFL | |
| PolyA tail | 100 nt | |

MRK_SLZF125

| SEQ ID NO: 65 consists of from 5′ end to 3′ end: 5′ UTR SEQ ID NO: 2, mRNA ORF SEQ ID | | 65 |
|---|---|---|
| NO: 66, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 66 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCAAU | |
| | AACAGAGCUAGGAGGGAGUUGCCUAGGUUUAUGAACUAC | |
| | ACUCUCAACAACGCGAAGAAAACCAAUGUGACGCUAUCC | |
| | AAGAAACGGAAGAGGAGGUUCCUGGGGUUUCUUUUAGGG | |
| | GUGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCUGACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUACGUUAACAAACAGGAAGGCAAAUCCCUUUAU | |
| | GUAAAGGGCGAGCCCAUCAUAAAUUUCUACGACCCACUU | |
| | GUGUUCCCCAGUUGCGAAUUCUGCGCAUCAAUCUCCCAG | |
| | GUGAACGAAAAGAUCAAUCAAUCCCUUGCUUUUAUACGA | |
| | AAGUCAGAUGAACUCCUGUCCGCCAUCGGUGGCUAUAUC | |
| | CCAGAAGCCCCAAGAGACGGACAAGCGUACGUCCGGAAA | |
| | GAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 67 |
| acid sequence | LSALRTGWYTSVITIELSNIKENKCNGTDAKVKLIKQELDKYK | |
| | NAVTELQLLMQSTPATNNRARRELPRFMNYTLNNAKKTNVT | |
| | LSKKRKRRFLGFLLGVGSAIASGVAVCKVLHLEGEVNKIKSAL | |
| | LSTNKAVVSLSNGVSVLTFKVLDLKNYIDKQLLPILNKQSCSIS | |
| | NIETVIEFQQKNNRLLEITREFSVNAGVTTPVSTYMLTNSELLS | |
| | LINDMPITNDQKKLMSNNVQIVRQQSYSIMCIIKEEVLAYVVQ | |
| | LPLYGVIDTPCWKLHTSPLCTTNTKEGSNICLTRTDRGWYCD | |
| | NAGSVSFFPQAETCKVQSNRVFCDTMNSLTLPSEVNLCNVDIF | |
| | NPKYDCKIMTSKTDVSSSVITSLGAIVSCYGKTKCTASNKNRG | |
| | IIKTFSNGCDYVSNKGVDTVSVGNTLYYVNKQEGKSLYVKGE | |
| | PIINFYDPLVFPSCEFCASISQVNEKINQSLAFIRKSDELLSAIGG | |
| | YIPEAPRDGQAYVRKDGEWVLLSTFL | |
| PolyA tail | 100 nt | |

MRK_sLZF111

| SEQ ID NO: 68 consists of from 5′ end to 3′ end: 5′ UTR SEQ ID NO: 2, mRNA ORF SEQ ID | | 68 |
|---|---|---|
| NO: 69, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 69 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGGGAGGCGGUAGCGGUGGCGGA | |
| | UCCGGCGGUGGAAGCGGCUCUGCCAUUGCUUCCGGCGUG | |
| | GCUGUAUGUAAAGUUCUCCACCUCGAGGGAGAGGUUAAU | |
| | AAGAUUAAGUCGGCCCUGCUGAGUACUAACAAAGCAGUG | |
| | GUGUCGCUGAGUAACGGAGUAAGUGUGUUAACAUUUAAG | |
| | GUGCUGGACCUCAAGAAUUAUAUUGACAAACAGUUGCUU | |
| | CCUAUUCUAAACAAACAGAGCUGUUCAAUAAGUAAUAUU | |
| | GAAACUGUUAUUGAGUUUCAGCAGAAGAACAACAGGCUU | |
| | CUUGAGAUUACACGCGAGUUCAGUGUCAAUGCCGGCGUU | |
| | ACAACACCCGUGUCUACCUACAUGCUGACGAAUUCUGAG | |
| | CUUCUCUCUCUCAUAAACGACAUGCCCAUUACGAAUGAC | |
| | CAAAAGAAACUUAUGUCCAACAACGUGCAGAUUGUGCGA | |
| | CAGCAAUCCUAUAGCAUUAUGUGUAUCAUCAAGGAAGAG | |
| | GUACUCGCUUAUGUUGUGCAGCUACCACUCUAUGGUGUG | |
| | AUUGACACCCCCUGUUGGAAGCUGCAUACCAGUCCACUC | |
| | UGCACCACUAACACAAAGGAAGGGAGCAAUAUUUGCCUC | |
| | ACUCGAACCGACAGGGGGUGGUAUUGCGAUAAUGCGGGC | |
| | UCCGUGUCCUUCUUUCCACAGGCUGAAACUUGUAAGGUA | |
| | CAGUCAAACCGCGUGUUCUGUGAUACUAUGAAUUCUCUG | |
| | ACUCUUCCCAGCGAGGUUAAUCUCUGCAACGUCGACAUU | |
| | UUCAAUCCUAAAUAUGACUGCAAGAUCAUGACCAGCAAG | |
| | ACCGACGUCUCCAGCUCAGUAAUCACUAGCCUAGGGGCC | |
| | AUUGUAAGCUGCUAUGGCAAAACCAAGUGUACUGCCUCU | |
| | AAUAAGAACAGAGGCAUAAUUAAAACCUUUUCAAAUGGC | |
| | UGUGACUAUGUGUCGAAUAAGGGCGUCGACACGGUCUCA | |
| | GUAGGGAAUACCCUCUACUACGUUAACAAACAGGAAGGC | |
| | AAAUCCCUUUAUGUAAAGGGCGAGCCCAUCAUAAAUUUC | |
| | UACGACCCACUUGUGUUCCCCAGUUGCGAAUUCUGCGCA | |
| | UCAAUCUCCCAGGUGAACGAAAAGAUCAAUCAAUCCCUU | |
| | GCUUUUAUACGAAAGUCAGAUGAACUCCUGUCCGCCAUC | |
| | GGUGGCUAUAUCCCAGAAGCCCCAAGAGACGGACAAGCG | |
| | UACGUCCGGAAAGAUGGUGAGUGGGUCCUCCUCUCUACC | |
| | UUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 70 |
| acid sequence | LSALRTGWYTSVITIELSNIKENKCNGTDAKVKLIKQELDKYK | |
| | NAVTELQLLMGGGSGGGSGGGSGSAIASGVAVCKVLHLEGE | |
| | VNKIKSALLSTNKAVVSLSNGVSVLTFKVLDLKNYIDKQLLPI | |
| | LNKQSCSISNIETVIEFQQKNNRLLEITREFSVNAGVTTPVSTY | |
| | MLTNSELLSLINDMPITNDQKKLMSNNVQIVRQQSYSIMCIIKE | |
| | EVLAYVVQLPLYGVIDTPCWKLHTSPLCTTNTKEGSNICLTRT | |
| | DRGWYCDNAGSVSFFPQAETCKVQSNRVFCDTMNSLTLPSEV | |
| | NLCNVDIFNPKYDCKIMTSKTDVSSSVITSLGAIVSCYGKTKCT | |
| | ASNKNRGIIKTFSNGCDYVSNKGVDTVSVGNTLYYVNKQEGK | |
| | SLYVKGEPIINFYDPLVFPSCEFCASISQVNEKINQSLAFIRKSD | |
| | ELLSAIGGYIPEAPRDGQAYVRKDGEWVLLSTFL | |
| PolyA tail | 100 nt | |

MRK_mLZF125

| SEQ ID NO: 71 consists of from 5′ end to 3′ end: 5′ UTR SEQ ID NO: 2, mRNA ORF SEQ ID | | 71 |
|---|---|---|
| NO: 72, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 72 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCAAU | |
| | AACAGAGCUAGGAGGGAGUUGCCUAGGUUUAUGAACUAC | |
| | ACUCUCAACAACGCGAAGAAAACCAAUGUGACGCUAUCC | |
| | AAGAAACGGAAGAGGAGGUUCCUGGGGUUUUUUUAGGG | |
| | GUGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCUGACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUACGUUAACAAACAGGAAGGCAAAUCCCUUUAU | |
| | GUAAAGGGCGAGCCCAUCAUAAAUUUCUACGACCCACUU | |
| | GUGUUCCCCAGUUGCGAAUUCUGCGCAUCAAUCUCCCAG | |
| | GUGAACGAAAAGAUCAAUCAAUCCCUUGCUUUUAUACGA | |
| | AAGUCAGAUGAACUCCUGCAUAACGUGAAUGCUGGGAAA | |
| | UCUACAACCAACAUCAUGAUCACUACCAUCAUUAUUGUG | |
| | AUUAUCGUAAUUCUGCUAUCCUUGAUUGCUGUCGGGCUG | |
| | CUUCUGUACUGUAAGGCCAGAUCGACGCCUGUGACCCUU | |
| | UCAAAAGACCAACUUAGCGGUAUCAAUAAUAUUGCCUUU | |
| | AGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGGGC | |
| Corresponding amino | MELLILKANAITTILTAVTFCFASGQNITEEFYQSTCSAVSKGY | 73 |
| acid sequence | LSALRTGWYTSVITIELSNIKENKCNGTDAKVKLIKQELDKYK | |
| | NAVTELQLLMQSTPATNNRARRELPRFMNYTLNNAKKTNVT | |
| | LSKKRKRRFLGFLLGVGSAIASGVAVCKVLHLEGEVNKIKSAL | |
| | LSTNKAVVSLSNGVSVLTFKVLDLKNYIDKQLLPILNKQSCSIS | |
| | NIETVIEFQQKNNRLLEITREFSVNAGVTTPVSTYMLTNSELLS | |
| | LINDMPITNDQKKLMSNNVQIVRQQSYSIMCIIKEEVLAYVVQ | |
| | LPLYGVIDTPCWKLHTSPLCTTNTKEGSNICLTRTDRGWYCD | |
| | NAGSVSFFPQAETCKVQSNRVFCDTMNSLTLPSEVNLCNVDIF | |
| | NPKYDCKIMTSKTDVSSSVITSLGAIVSCYGKTKCTASNKNRG | |
| | IIKTFSNGCDYVSNKGVDTVSVGNTLYYVNKQEGKSLYVKGE | |
| | PIINFYDPLVFPSCEFCASISQVNEKINQSLAFIRKSDELLHNVN | |
| | AGKSTTNIMITTIIIVIIVILLSLIAVGLLLYCKARSTPVTLSKDQ | |
| | LSGINNIAFSN | |
| PolyA tail | 100 nt | |

MRK_mLZF111

| SEQ ID NO: 74 consists of from 5′ end to 3′ end: 5′ UTR SEQ ID NO: 2, mRNA ORF SEQ ID | | 74 |
|---|---|---|
| NO: 75, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 75 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGGGAGGCGGUAGCGGUGGCGGA | |
| | UCCGGCGGUGGAAGCGGCUCUGCCAUUGCUUCCGGCGUG | |
| | GCUGUAUGUAAAGUUCUCCACCUCGAGGGAGAGGUUAAU | |
| | AAGAUUAAGUCGGCCCUGCUGAGUACUAACAAAGCAGUG | |
| | GUGUCGCUGAGUAACGGAGUAAGUGUGUUAACAUUUAAG | |
| | GUGCUGGACCUCAAGAAUUAUAUUGACAAACAGUUGCUU | |
| | CCUAUUCUAAACAAACAGAGCUGUUCAAUAAGUAAUAUU | |
| | GAAACUGUUAUUGAGUUUCAGCAGAAGAACAACAGGCUU | |
| | CUUGAGAUUACACGCGAGUUCAGUGUCAAUGCCGGCGUU | |
| | ACAACACCCGUGUCUACCUACAUGCUGACGAAUUCUGAG | |
| | CUUCUCUCUCUCAUAAACGACAUGCCCAUUACGAAUGAC | |
| | CAAAAGAAACUUAUGUCCAACAACGUGCAGAUUGUGCGA | |
| | CAGCAAUCCUAUAGCAUUAUGUGUAUCAUCAAGGAAGAG | |
| | GUACUCGCUUAUGUUGUGCAGCUACCACUCUAUGGUGUG | |
| | AUUGACACCCCCUGUUGGAAGCUGCAUACCAGUCCACUC | |
| | UGCACCACUAACACAAAGGAAGGGAGCAAUAUUUGCCUC | |
| | ACUCGAACCGACAGGGGGUGGUAUUGCGAUAAUGCGGGC | |
| | UCCGUGUCCUUCUUUCCACAGGCUGAAACUUGUAAGGUA | |
| | CAGUCAAACCGCGUGUUCUGUGAUACUAUGAAUUCUCUG | |
| | ACUCUUCCCAGCGAGGUUAAUCUCUGCAACGUCGACAUU | |
| | UUCAAUCCUAAAUAUGACUGCAAGAUCAUGACCAGCAAG | |
| | ACCGACGUCUCCAGCUCAGUAAUCACUAGCCUAGGGGCC | |
| | AUUGUAAGCUGCUAUGGCAAAACCAAGUGUACUGCCUCU | |
| | AAUAAGAACAGAGGCAUAAUUAAAACCUUUUCAAAUGGC | |
| | UGUGACUAUGUGUCGAAUAAGGGCGUCGACACGGUCUCA | |
| | GUAGGGAAUACCCUCUACUACGUUAACAAACAGGAAGGC | |
| | AAAUCCCUUUAUGUAAAGGGCGAGCCCAUCAUAAAUUUC | |
| | UACGACCCACUUGUGUUCCCCAGUUGCGAAUUCUGCGCA | |
| | UCAAUCUCCCAGGUGAACGAAAAGAUCAAUCAAUCCCUU | |
| | GCUUUUAUACGAAAGUCAGAUGAACUCCUGCAUAACGUG | |
| | AAUGCUGGGAAAUCUACAACCAACAUCAUGAUCACUACC | |
| | AUCAUUAUUGUGAUUAUCGUAAUUCUGCUAUCCUUGAUU | |
| | GCUGUCGGGCUGCUUCUGUACUGUAAGGCCAGAUCGACG | |
| | CCUGUGACCCUUUCAAAAGACCAACUUAGCGGUAUCAAU | |
| | AAUAUUGCCUUUAGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 76 |
| acid sequence | LSALRTGWYTSVITIELSNIKENKCNGTDAKVKLIKQELDKYK | |
| | NAVTELQLLMGGGSGGGSGGGSGSAIASGVAVCKVLHLEGE | |
| | VNKIKSALLSTNKAVVSLSNGVSVLTFKVLDLKNYIDKQLLPI | |
| | LNKQSCSISNIETVIEFQQKNNRLLEITREFSVNAGVTTPVSTY | |
| | MLTNSELLSLINDMPITNDQKKLMSNNVQIVRQQSYSIMCIIKE | |
| | EVLAYVVQLPLYGVIDTPCWKLHTSPLCTTNTKEGSNICLTRT | |
| | DRGWYCDNAGSVSFFPQAETCKVQSNRVFCDTMNSLTLPSEV | |
| | NLCNVDIFNPKYDCKIMTSKTDVSSSVITSLGAIVSCYGKTKCT | |
| | ASNKNRGIIKTFSNGCDYVSNKGVDTVSVGNTLYYVNKQEGK | |
| | SLYVKGEPIINFYDPLVFPSCEFCASISQVNEKINQSLAFIRKSD | |
| | ELLHNVNAGKSTTNIMITTIIIVIIVILLSLIAVGLLLYCKARSTP | |
| | VTLSKDQLSGINNIAFSN | |
| PolyA tail | 100 nt | |

MRK-04 membrane-bound DS-CAV1 (stabilized prefusion F protein)

| SEQ ID NO: 90 consists of from 5′ end to 3′ end: 5′ UTR SEQ ID NO: 2, mRNA ORF SEQ ID | | 90 |
|---|---|---|
| NO: 88, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 88 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAAAACGCUGUGACC | |
| | GAGCUGCAGUUAUUGAUGCAGAGUACACCUGCCACCAAU | |
| | AACAGAGCUAGGAGGGAGUUGCCUAGGUUUAUGAACUAC | |
| | ACUCUCAACAACGCGAAAAAAACCAAUGUGACGCUAUCC | |
| | AAGAAACGGAAGAGGAGGUUCCUGGGGUUUCUUUUAGGG | |
| | GUGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAAAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCUGACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUACGUUAACAAACAGGAAGGCAAAUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGCAUAACGUGAAUGCUGGGAAA | |
| | UCUACAACCAACAUCAUGAUCACUACCAUCAUUAUUGUG | |
| | AUUAUCGUAAUUCUGCUAUCCUUGAUUGCUGUCGGGCUG | |
| | CUUCUGUACUGUAAGGCCAGAUCGACGCCUGUGACCCUU | |
| | UCAAAAGACCAACUUAGCGGUAUCAAUAAUAUUGCCUUU | |
| | AGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 89 |
| acid sequence | LSALRTGWYTSVITIELSNIKENKCNGTDAKVKLIKQELDKYK | |
| | NAVTELQLLMQSTPATNNRARRELPRFMNYTLNNAKKTNVT | |
| | LSKKRKRRFLGFLLGVGSAIASGVAVCKVLHLEGEVNKIKSAL | |
| | LSTNKAVVSLSNGVSVLTFKVLDLKNYIDKQLLPILNKQSCSIS | |
| | NIETVIEFQQKNNRLLEITREFSVNAGVTTPVSTYMLTNSELLS | |
| | LINDMPITNDQKKLMSNNVQIVRQQSYSIMCIIKEEVLAYVVQ | |
| | LPLYGVIDTPCWKLHTSPLCTTNTKEGSNICLTRTDRGWYCD | |
| | NAGSVSFFPQAETCKVQSNRVFCDTMNSLTLPSEVNLCNVDIF | |
| | NPKYDCKIMTSKTDVSSSVITSLGAIVSCYGKTKCTASNKNRG | |
| | IIKTFSNGCDYVSNKGVDTVSVGNTLYYVNKQEGKSLYVKGE | |
| | PIINFYDPLVFPSDEFDASISQVNEKINQSLAFIRKSDELLHNVN | |
| | AGKSTTNIMITTIIIVIIVILLSLIAVGLLLYCKARSTPVTLSKDQ | |
| | LSGINNIAFSN | |
| PolyA tail | 100 nt | |

MRK_04_nopolyA_3mut

| SEQ ID NO: 92 consists of from 5′ end to 3′ end: 5′ UTR SEQ ID NO: 2, mRNA ORF SEQ ID | | 92 |
|---|---|---|
| NO: 91, and 3′ UTR SEQ ID NO: 4. | | |
| Chemistry | 1-methylpseudouridine | |
| Cap | 7mG(5′)ppp(5′)NImpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 91 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGAGUGCGCUCCGCACAGGAUGG | |
| | UACACCUCCGUGAUCACCAUCGAGCUCAGCAAUAUUAAA | |
| | GAGAACAAGUGCAAUGGUACCGACGCUAAAGUCAAACUU | |
| | AUCAAGCAGGAACUCGACAAAUAUAAGAACGCUGUGACC | |
| | GAGCUGCAGUUAUUGAUGCAGAGUACACCUGCCACCAAU | |
| | AACAGAGCUAGGAGGGAGUUGCCUAGGUUUAUGAACUAC | |
| | ACUCUCAACAACGCGAAGAAAACCAAUGUGACGCUAUCC | |
| | AAGAAACGGAAGAGGAGGUUCCUGGGGUUUCUUUUAGGG | |
| | GUGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUAAGUAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCUGACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUACGUUAACAAACAGGAAGGCAAAUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGCAUAACGUGAAUGCUGGGAAA | |
| | UCUACAACCAACAUCAUGAUCACUACCAUCAUUAUUGUG | |
| | AUUAUCGUAAUUCUGCUAUCCUUGAUUGCUGUCGGGCUG | |
| | CUUCUGUACUGUAAGGCCAGAUCGACGCCUGUGACCCUU | |
| | UCAAAAGACCAACUUAGCGGUAUCAAUAAUAUUGCCUUU | |
| | AGCAAU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 89 |
| acid sequence | LSALRTGWYTSVITIELSNIKENKCNGTDAKVKLIKQELDKYK | |
| | NAVTELQLLMQSTPATNNRARRELPRFMNYTLNNAKKTNVT | |
| | LSKKRKRRFLGFLLGVGSAIASGVAVCKVLHLEGEVNKIKSAL | |
| | LSTNKAVVSLSNGVSVLTFKVLDLKNYIDKQLLPILNKQSCSIS | |
| | NIETVIEFQQKNNRLLEITREFSVNAGVTTPVSTYMLTNSELLS | |
| | LINDMPITNDQKKLMSNNVQIVRQQSYSIMCIIKEEVLAYVVQ | |
| | LPLYGVIDTPCWKLHTSPLCTTNTKEGSNICLTRTDRGWYCD | |
| | NAGSVSFFPQAETCKVQSNRVFCDTMNSLTLPSEVNLCNVDIF | |
| | NPKYDCKIMTSKTDVSSSVITSLGAIVSCYGKTKCTASNKNRG | |
| | IIKTFSNGCDYVSNKGVDTVSVGNTLYYVNKQEGKSLYVKGE | |
| | PIINFYDPLVFPSDEFDASISQVNEKINQSLAFIRKSDELLHNVN | |
| | AGKSTTNIMITTIIIVIIVILLSLIAVGLLLYCKARSTPVTLSKDQ | |
| | LSGINNIAFSN | |

VRC-4A

| SEQ ID NO: 98 consists of from 5′ end to 3′ end: 5′ UTR SEQ ID NO: 2, mRNA ORF SEQ ID | | 98 |
|---|---|---|
| NO: 96, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 96 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGGGCGCGCUCCGCACAGGAUGG | |
| | UACACCUCCGUGAUCACCAUCGAGCUCAGCAAUAUUAAA | |
| | GAGAUCAAGUGCAAUGGUACCGACGCUAAAGUCAAACUU | |
| | AUCAAGCAGGAACUCGACAAAUAUAAGAACGCUGUGACC | |
| | GACCUGCAGUUAUUGAUGCAGAGUACACCUGCCACCGGG | |
| | UCGGGCUCUGCCAUUUGCUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | AACGGAGUAAGUGUGUUAACAUUUAAGGUGCUGGACCUC | |
| | AAGAAUUAUAUUGACAAACAGUUGCUUCCUAUUCUAAAC | |
| | AAACAGAGCUGUUCAAUACCCAAUAUUGAAACUGUUAUU | |
| | GAGUUUCAGCAGAAGAACAACAGGCUUCUUGAGAUUACA | |
| | CGCGAGUUCAGUGUCAAUGCCGGCGUUACAACACCCGUG | |
| | UCUACCUACAUGCUGACGAAUUCUGAGCUUCUCUCUCUC | |
| | AUAAACGACAUGCCCAUUACGAAUGACCAAAAGAAACUU | |
| | AUGUCCAACAACGUGCAGAUUGUGCGACAGCAAUCCUAU | |
| | AGCAUUAUGUGUAUCAUCAAGGAAGAGGUACUCGCUUAU | |
| | GUUGUGCAGCUACCACUCUAUGGUGUGAUUGACACCCCC | |
| | UGUUGGAAGCUGCAUACCAGUCCACUCUGCACCACUAAC | |
| | ACAAAGGAAGGGAGCAAUAUUUGCCUCACUCGAACCGAC | |
| | AGGGGGUGGUAUUGCGAUAAUGCGGGCUCCGUGUCCUUC | |
| | UUUCCACAGGCUGAAACUUGUAAGGUACAGUCAAACCGC | |
| | GUGUUCUGUGAUACUAUGAAUUCUCGUACUCUUCCCAGC | |
| | GAGGUUAAUCUCUGCAACGUCGACAUUUUCAAUCCUAAA | |
| | UAUGACUGCAAGAUCAUGACCAGCAAGACCGACGUCUCC | |
| | AGCUCAGUAAUCACUAGCCUAGGGGCCAUUGUAAGCUGC | |
| | UAUGGCAAAACCAAGUGUACUGCCUCUAAUAAGAACAGA | |
| | GGCAUAAUUAAAACCUUUUCAAAUGGCUGUGACUAUGUG | |
| | UCGAAUAAGGGCGUCGACACGGUCUCAGUAGGGAAUACC | |
| | CUCUACUGCGUUAACAAACAGGAAGGCCAGUCCCUUUAU | |
| | GUAAAGGGCGAGCCCAUCAUAAAUUUCUACGACCCACUU | |
| | GUGUUCCCCAGUGAUGAAUUCGAUGCAUCAAUCUCCCAG | |
| | GUGAACGAAAAGAUCAAUCAAUCCCUUGCUUUUAUACGA | |
| | AAGUCAGAUGAACUCCUGUCCGCCAUCGGUGGCUAUAUC | |
| | CCAGAAGCCCCAAGAGACGGACAAGCGUACGUCCGGAAA | |
| | GAUGGUGAGUGGGUCCUCCUCUCUACCUUUCUU | |
| 3′ UTR | UGAUAAUAGGCUGGAGCCUCGGUGGCCAUGCUUCUUGCC | |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 44 |
| acid sequence | LGALRTGWYTSVITIELSNIKEIKCNGTDAKVKLIKQELDKYK | |
| | NAVTDLQLLMQSTPATGSGSAICSGVAVCKVLHLEGEVNKIK | |
| | SALLSTNKAVVSLSNGVSVLTFKVLDLKNYIDKQLLPILNKQS | |
| | CSIPNIETVIEFQQKNNRLLEITREFSVNAGVTTPVSTYMLTNSE | |
| | LLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | NRGIIKTFSNGCDYVSNKGVDTVSVGNTLYCVNKQEGQSLYV | |
| | KGEPIINFYDPLVFPSDEFDASISQVNEKINQSLAFIRKSDELLS | |
| | AIGGYIPEAPRDGQAYVRKDGEWVLLSTFL | |
| PolyA tail | 100 nt | |

VRC-5A

| SEQ ID NO: 99 consists of from 5′ end to 3′ end: 5′ UTR SEQ ID NO: 2, mRNA ORF SEQ ID | | 99 |
|---|---|---|
| NO: 97, and 3′ UTR SEQ ID NO: 4. | | |
| Chemistry | 1-methylpseudouridine | |
| Cap | 7mG(5′)ppp(5′)NlmpNp | |
| 5′ UTR | GGGAAAUAAGAGAGAAAAGAAGAGUAAGAAGAAAUAUA | 2 |
| | AGAGCCACC | |
| ORF of mRNA | AUGGAACUGCUCAUUUUGAAGGCAAACGCUAUCACGACA | 97 |
| Construct | AUACUCACUGCAGUGACCUUCUGUUUUGCCUCAGGCCAG | |
| (excluding the stop | AACAUAACCGAGGAGUUUUAUCAAUCUACAUGCAGCGCU | |
| codon) | GUAUCUAAAGGCUACCUGGGCGCGCUCCGCACAGGAUGG | |
| | UACACCUCCGUGAUCACCAUCGAGCUCAGCAAUAUUAAA | |
| | GAGAUCAAGUGCAAUGGUACCGACGCUAAAGUCAAACUU | |
| | AUCAAGCAGGAACUCGACAAAUAUAAGAACGCUGUGACC | |
| | GACCUGCAGUUAUUGAUGCAGAGUACACCUGCCACCGGG | |
| | UCGGGCUCUGCCAUUGCUUCCGGCGUGGCUGUAUGUAAA | |
| | GUUCUCCACCUCGAGGGAGAGGUUAAUAAGAUUAAGUCG | |
| | GCCCUGCUGAGUACUAACAAAGCAGUGGUGUCGCUGAGU | |
| | GGCUGCGGAGUAAGUGUGUUAACAUUUAAGGUGCUGGAC | |
| | CUCAAGAAUUAUAUUGACAAACAGUUGCUUCCUAUUCUA | |
| | AACAAACAGAGCUGUUCAAUACCCAAUAUUGAAACUGUU | |
| | AUUGAGUUUCAGCAGAAGAACAACAGGCUUCUUGAGAUU | |
| | ACACGCGAGUUCAGUGUCAAUGCCGGCGUUACAACACCC | |
| | GUGUCUACCUACAUGCUGACGAAUUCUGAGCUUCUCUCU | |
| | CUCAUAAACGACAUGCCCAUUACGAAUGACCAAAAGAAA | |
| | CUUAUGUCCAACAACGUGCAGAUUGUGCGACAGCAAUCC | |
| | UAUAGCAUUAUGUGUAUCAUCAAGGAAGAGGUACUCGCU | |
| | UAUGUUGUGCAGCUACCACUCUAUGGUGUGAUUGACACC | |
| | CCCUGUUGGAAGCUGCAUACCAGUCCACUCUGCACCACUA | |
| | ACACAAAGGAAGGGAGCAAUAUUUGCCUCACUCGAACCG | |
| | ACAGGGGGUGGUAUUGCGAUAAUGCGGGCUCCGUGUCCU | |
| | UCUUUCCACAGGCUGAAACUUGUAAGGUACAGUCAAACC | |
| | GCGUGUUCUGUGAUACUAUGAAUUCUCGUACUCUUCCCA | |
| | GCGAGGUUAAUCUCUGCAACGUCGACAUUUUCAAUCCUA | |
| | AAUAUGACUGCAAGAUCAUGACCAGCAAGACCGACGUCU | |
| | CCAGCUCAGUAAUCACUAGCCUAGGGGCCAUUGUAAGCU | |
| | GCUAUGGCAAAACCAAGUGUACUGCCUCUAAUAAGUGCA | |
| | GAGGCAUAAUUAAAACCUUUUCAAAUGGCUGUGACUAUG | |
| | UGUCGAAUAAGGGCGUCGACACGGUCUCAGUAGGGAAUA | |
| | CCCUCUACUACGUUAACAAACAGGAAGGCCAGUCCCUUU | |
| | AUGUAAAGGGCGAGCCCAUCAUAAAUUUCUACGACCCAC | |
| | UUGUGUUCCCCAGUGAUGAAUUCGAUGCAUCAAUCUCCC | |
| | AGGUGAACGAAAAGAUCAAUCAAUCCCUUGCUUUUAUAC | |
| | GAAAGUCAGAUGAACUCCUGUCCGCCAUCGGUGGCUAUA | |
| | UCCCAGAAGCCCCAAGAGACGGACAAGCGUACGUCCGGA | |
| | AAGAUGGUGAGUGGGUCCUCCUCUCUACCUUUCUU | |
| 3′ UTR | UGAUAAUAGGCUGGAGCCUCGGUGGCCAUGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGGC | |
| Corresponding amino | MELLILKANAITTILTAVTFCFASGQNITEEFYQSTCSAVSKGY | 47 |
| acid sequence | LGALRTGWYTSVITIELSNIKEIKCNGTDAKVKLIKQELDKYK | |
| | NAVTDLQLLMQSTPATGSGSAIASGVAVCKVLHLEGEVNKIK | |
| | SALLSTNKAVVSLSGCGVSVLTFKVLDLKNYIDKQLLPILNKQ | |
| | SCSIPNIETVIEFQQKNNRLLEITREFSVNAGVTTPVSTYMLTNS | |
| | ELLSLINDMPITNDQKKLMSNNVQIVRQQSYSIMCIIKEEVLAY | |
| | VVQLPLYGVIDTPCWKLHTSPLCTTNTKEGSNICLTRTDRGW | |
| | YCDNAGSVSFFPQAETCKVQSNRVFCDTMNSRTLPSEVNLCN | |
| | VDIFNPKYDCKIMTSKTDVSSSVITSLGAIVSCYGKTKCTASNK | |
| | CRGIIKTFSNGCDYVSNKGVDTVSVGNTLYYVNKQEGQSLYV | |
| | KGEPIINFYDPLVFPSDEFDASISQVNEKINQSLAFIRKSDELLS | |
| | AIGGYIPEAPRDGQAYVRKDGEWVLLSTFL | |
| PolyA tail | 100 nt | |

*The underlined sequences above are signal sequences. It should be understood that any one of the
open reading frames and/or corresponding amino acid sequences described in Table 7 may include or
exclude the underlined signal sequence. It should also be understood that the underlined sequence
may be replaced by a different signal sequence, for example, any one of SEQ ID NOs: 79-84.

### EQUIVALENTS

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

The entire contents of International Application Nos. PCT/US2015/02740, PCT/US2016/043348, PCT/US2016/043332, PCT/US2016/058327, PCT/US2016/058324, PCT/US2016/058314, PCT/US2016/058310, PCT/US2016/058321, PCT/US2016/058297, PCT/US2016/058319, and PCT/US2016/058314 are incorporated herein by reference.

1.-33. (canceled)

34. A respiratory syncytial virus (RSV) messenger ribonucleic acid (mRNA) vaccine composition, comprising:
an mRNA comprising an open reading frame (ORF) encoding an RSV Fusion (F) glycoprotein comprising A149C, S155C, S190F, V207L, S290C, and Y458C amino acid substitutions relative to a wild-type RSV F glycoprotein, wherein the RSV F glycoprotein comprises a F1 subunit linked to a F2 subunit through a linker consisting of glycine and serine, and wherein the ORF comprises nucleosides consisting of N1-methylpseudouridine, adenosine, guanosine, and cytidine,
formulated in a lipid nanoparticle comprising 20-60 mol % ionizable cationic lipid, 5-25 mol % neutral lipid, 25-55 mol % sterol, and 0.5-15 mol % PEG-modified lipid.

35. The RSV mRNA vaccine composition of claim 34, wherein the lipid nanoparticle comprises 40-50 mol % ionizable cationic lipid, 10-20 mol % neutral lipid, 35-45 mol % cholesterol, and 1-5 mol % PEG-modified lipid.

36. The RSV mRNA vaccine composition of claim 35, wherein the ionizable cationic lipid is a compound of Formula (I): 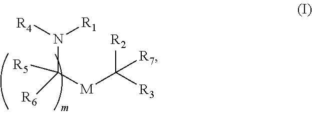
or a salt thereof, wherein:
R₁ is selected from the group consisting of C₅₋₃₀ alkyl, C₅₋₂₀ alkenyl, and —R″M′R′;
R₂ and R₃ are independently selected from the group consisting of C₁₋₁₄ alkyl and C₂₋₁₄ alkenyl;
R₄ is —(CH₂)ₙQ, wherein Q is —OR, and n is selected from 1, 2, 3, 4, and 5;
each R₅ is H;
each R₆ is H;
M and M′ are independently selected from —C(O)O— and —OC(O)—;
R₇ is H;
R is H;
R′ is selected from the group consisting of C₁₋₁₈ alkyl and C₂₋₁₈ alkenyl;
R″ is selected from the group consisting of C₃₋₁₄ alkyl and C₃₋₁₄ alkenyl; and
m is selected from 5, 6, 7, 8, 9, 10, 11, 12, and 13.

37. The RSV mRNA vaccine composition of claim 36, wherein:
R₁ is R″M′R′;
R₂ and R₃ are each independently C₁₋₁₄ alkyl;
R₄ is (CH₂)ₙQ, wherein Q is OH and n is 4;
M and M′ are each independently —OC(O)—;
R₅, R₆, and R₇ are each H;
R′ is C₁₋₁₂ alkyl substituted with C₆₋₉ alkyl;
R″ is C₃₋₁₄ alkyl; and
m is 6.

38. The RSV mRNA vaccine composition of claim 37, wherein:
R₁ is C₅₋₂₀ alkenyl;
R₂ and R₃ are each independently C₁₋₁₄ alkyl;
R₄ is (CH₂)ₙQ, wherein Q is OH and n is 3;
M is —C(O)O—;
R₅, R₆, and R₇ are each H; and
m is 6.

39. The RSV mRNA vaccine composition of claim 36, wherein the compound of Formula (I) comprises a compound of Formula (IId), 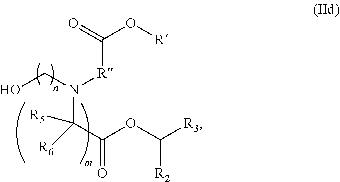
or a salt thereof, wherein n is 2, 3, or 4.

40. The RSV mRNA vaccine composition of claim 36, wherein the compound of Formula (I) is Compound 1:

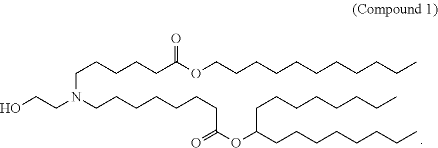 [SMILES: CCCCCCCCCCCOC(=O)CCCCCN(CCO)CCCCCCCC(=O)OC(CCCCCCCC)CCCCCCCC]

41. The RSV mRNA vaccine composition of claim 36, wherein the neutral lipid is 1,2 distearoyl-sn-glycero-3-phosphocholine (DSPC) and the PEG-modified lipid is 1,2 dimyristoyl-sn-glycerol, methoxypolyethyleneglycol (PEG2000 DMG).

42. A method comprising administering to a subject the RSV mRNA vaccine composition of claim 34 in a therapeutically effective amount to induce in the subject an RSV F protein neutralizing antibody titer.

43. The method of claim 42, wherein the vaccine is administered intramuscularly.


SEQUENCE LISTING (100 sequences)

SEQ ID NO: 1 - RNA, 1771 nt, synthetic construct
  misc_feature 1..1771: note = Synthetic Polynucleotide
  source 1..1771: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 2 - RNA, 47 nt, synthetic construct
  misc_feature 1..47: note = Synthetic Polynucleotide
  source 1..47: mol_type = other RNA
gggaaataagagagaaaagaagagtaagaagaaatataagagccacc

SEQ ID NO: 3 - RNA, 1605 nt, synthetic construct
  misc_feature 1..1605: note = Synthetic Polynucleotide
  source 1..1605: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 4 - RNA, 119 nt, synthetic construct
  misc_feature 1..119: note = Synthetic Polynucleotide
  source 1..119: mol_type = other RNA
tgataataggctggagcctcggtggccatgcttcttgccccttgggcctccccccagcccctcctccccttcctgcacccgtacccccgtggtctttgaataaagtctgagtgggcggc

SEQ ID NO: 5 - protein, 535 aa, synthetic construct
  REGION 1..535: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 6 - RNA, 1774 nt, synthetic construct
  misc_feature 1..1774: note = Synthetic Polynucleotide
  source 1..1774: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 7 - RNA, 1608 nt, synthetic construct
  misc_feature 1..1608: note = Synthetic Polynucleotide
  source 1..1608: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 8 - protein, 536 aa, synthetic construct
  REGION 1..536: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVCKVLHLEGEVNKIKSALLSTNKAVVSLSGCGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 9 - RNA, 1777 nt, synthetic construct
  misc_feature 1..1777: note = Synthetic Polynucleotide
  source 1..1777: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccaataacgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 10 - RNA, 1611 nt, synthetic construct
  misc_feature 1..1611: note = Synthetic Polynucleotide
  source 1..1611: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccaataacgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 11 - protein, 537 aa, synthetic construct
  REGION 1..537: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNGSGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 12 - RNA, 1681 nt, synthetic construct
  misc_feature 1..1681: note = Synthetic Polynucleotide
  source 1..1681: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 13 - RNA, 1515 nt, synthetic construct
  misc_feature 1..1515: note = Synthetic Polynucleotide
  source 1..1515: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 14 - protein, 505 aa, synthetic construct
  REGION 1..505: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAICSGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 15 - RNA, 1684 nt, synthetic construct
  misc_feature 1..1684: note = Synthetic Polynucleotide
  source 1..1684: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 16 - RNA, 1518 nt, synthetic construct
  misc_feature 1..1518: note = Synthetic Polynucleotide
  source 1..1518: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 17 - protein, 506 aa, synthetic construct
  REGION 1..506: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVCKVLHLEGEVNKIKSALLSTNKAVVSLSGCGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 18 - RNA, 1687 nt, synthetic construct
  misc_feature 1..1687: note = Synthetic Polynucleotide
  source 1..1687: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccaataacgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 19 - RNA, 1521 nt, synthetic construct
  misc_feature 1..1521: note = Synthetic Polynucleotide
  source 1..1521: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccaataacgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 20 - protein, 507 aa, synthetic construct
  REGION 1..507: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNGSGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 21 - RNA, 1771 nt, synthetic construct
  misc_feature 1..1771: note = Synthetic Polynucleotide
  source 1..1771: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 22 - RNA, 1605 nt, synthetic construct
  misc_feature 1..1605: note = Synthetic Polynucleotide
  source 1..1605: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 23 - RNA, 1774 nt, synthetic construct
  misc_feature 1..1774: note = Synthetic Polynucleotide
  source 1..1774: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 24 - RNA, 1608 nt, synthetic construct
  misc_feature 1..1608: note = Synthetic Polynucleotide
  source 1..1608: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 25 - RNA, 1777 nt, synthetic construct
  misc_feature 1..1777: note = Synthetic Polynucleotide
  source 1..1777: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 26 - RNA, 1611 nt, synthetic construct
  misc_feature 1..1611: note = Synthetic Polynucleotide
  source 1..1611: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 27 - RNA, 1681 nt, synthetic construct
  misc_feature 1..1681: note = Synthetic Polynucleotide
  source 1..1681: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 28 - RNA, 1515 nt, synthetic construct
  misc_feature 1..1515: note = Synthetic Polynucleotide
  source 1..1515: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 29 - RNA, 1684 nt, synthetic construct
  misc_feature 1..1684: note = Synthetic Polynucleotide
  source 1..1684: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 30 - RNA, 1518 nt, synthetic construct
  misc_feature 1..1518: note = Synthetic Polynucleotide
  source 1..1518: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 31 - RNA, 1687 nt, synthetic construct
  misc_feature 1..1687: note = Synthetic Polynucleotide
  source 1..1687: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 32 - RNA, 1521 nt, synthetic construct
  misc_feature 1..1521: note = Synthetic Polynucleotide
  source 1..1521: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 33 - RNA, 1771 nt, synthetic construct
  misc_feature 1..1771: note = Synthetic Polynucleotide
  source 1..1771: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagattaagtgcaatggtaccgacgctaaagtcaaacttatcaagcaggaactcgacaaatataagaacgctgtgaccgacctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 34 - RNA, 1605 nt, synthetic construct
  misc_feature 1..1605: note = Synthetic Polynucleotide
  source 1..1605: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagattaagtgcaatggtaccgacgctaaagtcaaacttatcaagcaggaactcgacaaatataagaacgctgtgaccgacctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 35 - protein, 535 aa, synthetic construct
  REGION 1..535: note = Synthetic Polypeptide
MELLILKANAITTILTAVTFCFASGQNITEEFYQSTCSAVSKGYLGALRTGWYTSVITIELSNIKEIKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 36 - RNA, 1774 nt, synthetic construct
  misc_feature 1..1774: note = Synthetic Polynucleotide
  source 1..1774: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagattaagtgcaatggtaccgacgctaaagtcaaacttatcaagcaggaactcgacaaatataagaacgctgtgaccgacctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 37 - RNA, 1608 nt, synthetic construct
  misc_feature 1..1608: note = Synthetic Polynucleotide
  source 1..1608: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagattaagtgcaatggtaccgacgctaaagtcaaacttatcaagcaggaactcgacaaatataagaacgctgtgaccgacctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 38 - protein, 536 aa, synthetic construct
  REGION 1..536: note = Synthetic Polypeptide
MELLILKANAITTILTAVTFCFASGQNITEEFYQSTCSAVSKGYLGALRTGWYTSVITIELSNIKEIKCNGTDAKVKLIKQELDKYKNAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 39 - RNA, 1771 nt, synthetic construct
  misc_feature 1..1771: note = Synthetic Polynucleotide
  source 1..1771: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 40 - RNA, 1605 nt, synthetic construct
  misc_feature 1..1605: note = Synthetic Polynucleotide
  source 1..1605: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 41 - protein, 535 aa, synthetic construct
  REGION 1..535: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 42 - RNA, 1681 nt, synthetic construct
  misc_feature 1..1681: note = Synthetic Polynucleotide
  source 1..1681: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagattaagtgcaatggtaccgacgctaaagtcaaacttatcaagcaggaactcgacaaatataagaacgctgtgaccgacctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 43 - RNA, 1515 nt, synthetic construct
  misc_feature 1..1515: note = Synthetic Polynucleotide
  source 1..1515: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagattaagtgcaatggtaccgacgctaaagtcaaacttatcaagcaggaactcgacaaatataagaacgctgtgaccgacctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 44 - protein, 505 aa, synthetic construct
  REGION 1..505: note = Synthetic Polypeptide
MELLILKANAITTILTAVTFCFASGQNITEEFYQSTCSAVSKGYLGALRTGWYTSVITIELSNIKEIKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLSAIGGYIPEAPRDGQAYVRKDGEWVLLSTFL

SEQ ID NO: 45 - RNA, 1684 nt, synthetic construct
  misc_feature 1..1684: note = Synthetic Polynucleotide
  source 1..1684: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagattaagtgcaatggtaccgacgctaaagtcaaacttatcaagcaggaactcgacaaatataagaacgctgtgaccgacctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 46 - RNA, 1518 nt, synthetic construct
  misc_feature 1..1518: note = Synthetic Polynucleotide
  source 1..1518: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagattaagtgcaatggtaccgacgctaaagtcaaacttatcaagcaggaactcgacaaatataagaacgctgtgaccgacctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 47 - protein, 506 aa, synthetic construct
  REGION 1..506: note = Synthetic Polypeptide
MELLILKANAITTILTAVTFCFASGQNITEEFYQSTCSAVSKGYLGALRTGWYTSVITIELSNIKEIKCNGTDAKVKLIKQELDKYKNAVTDLQLLMQSTPATGSGSAIASGVAVCKVLHLEGEVNKIKSALLSTNKAVVSLSGCGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKCRGIIKTFSNGCDYVSNKGVDTVSVGNTLYYVNKQEGQSLYVKGEPIINFYDPLVFPSDEFDASISQVNEKINQSLAFIRKSDELLSAIGGYIPEAPRDGQAYVRKDGEWVLLSTFL

SEQ ID NO: 48 - RNA, 1681 nt, synthetic construct
  misc_feature 1..1681: note = Synthetic Polynucleotide
  source 1..1681: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 49 - RNA, 1515 nt, synthetic construct
  misc_feature 1..1515: note = Synthetic Polynucleotide
  source 1..1515: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 50 - protein, 505 aa, synthetic construct
  REGION 1..505: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATGSGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFL

SEQ ID NO: 51 - RNA, 1781 nt, synthetic construct
  misc_feature 1..1781: note = Synthetic Polynucleotide
  source 1..1781: mol_type = other RNA
gggaaataagagagaaaagaagagtaagaagaaatataagaccccggcgccgccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggcctagcttcttgccccttgggcctccccccagcccctcctccccttcctgcacccgtacccccgtggtctttgaataaagtctgagtgggcggc

SEQ ID NO: 52 - RNA, 1605 nt, synthetic construct; identical to SEQ ID NO: 22
  misc_feature 1..1605: note = Synthetic Polynucleotide
  source 1..1605: mol_type = other RNA

SEQ ID NO: 53 - RNA, 1781 nt, synthetic construct
  misc_feature 1..1781: note = Synthetic Polynucleotide
  source 1..1781: mol_type = other RNA
gggaaataagagagaaaagaagagtaagaagaaatataagaccccggcgccgccaccatggagctgctgatcctcaaggctaacgccatcaccaccatcctgaccgccgtgaccttctgcttcgccagcgggcagaacatcaccgaggagttctaccagagcacttgcagcgccgtgagcaagggctacctgtcagccctgcggaccggctggtacaccagcgtgatcaccatcgagctgagcaacatcaaggagaacaagtgcaacggcaccgacgccaaggtgaagctgatcaagcaggagctggacaagtacaagaacgccgtgaccgagctgcagctgctgatgcagtccacgcccgctaccgggtcaggcagcgccatctgctccggcgtggccgtgtgcaaggtgctgcacctggagggcgaggtgaacaagatcaagagcgccctgctgagcaccaacaaggccgtggtgagcctgagcaacggcgtgagcgtgctgaccttcaaggtgctggacctgaagaactacatcgacaagcagctgctgcccatcctgaacaagcagtcctgctccatctccaacatcgaaactgtgatcgagttccagcagaagaacaaccgcctgctggagatcacccgcgagttcagcgtgaacgccggcgtgaccaccccagtgagcacctacatgctgaccaacagcgagctgctgagcctgatcaacgacatgcccatcaccaacgaccagaagaagctgatgagcaacaacgtgcagatcgtgcgccagcagtcctactctatcatgtgcatcatcaaggaggaggtgctggcctacgtggtgcagctgcccctgtacggcgtgatcgacacaccctgctggaagctgcacacctcccctctgtgcaccactaacaccaaggaggggagcaacatctgcctgacccggaccgaccgcggctggtactgcgacaacgccggcagcgtgagcttcttccctcaggccgaaacctgcaaggtgcagagcaacagggtgttctgcgacaccatgaactcccggaccctgcccagcgaggtgaacctgtgcaacgtggacatcttcaaccccaagtacgattgcaagatcatgacctccaagaccgacgtgagcagctccgtgatcacctccctgggcgccatcgtgagttgctacggcaagaccaagtgcaccgccagcaacaagaaccgcggcatcatcaagaccttcagcaacggctgcgactacgtgtccaacaagggcgtggacaccgtgagcgtgggcaacaccctgtactgcgtgaacaagcaggagggcaagagcctgtacgtgaagggcgagcccatcatcaacttctacgatccgctggtgttcccctccgacgagttcgacgcctccatcagccaggtgaacgagaagatcaaccagagcctggccttcatccgcaagtccgacgagctgctgcacaacgtcaacgccggtaagagcaccaccaacatcatgatcaccacaatcatcatcgtgatcattgtgatcttgctctccttaatcgccgtgggcctgctgctgtactgcaaggcccggagcaccccagtgaccctgagcaaagatcagctgtccggcatcaacaacatcgccttctccaactgataataggctggagcctcggtggcctagcttcttgccccttgggcctccccccagcccctcctccccttcctgcacccgtacccccgtggtctttgaataaagtctgagtgggcggc

SEQ ID NO: 54 - RNA, 1605 nt, synthetic construct
  misc_feature 1..1605: note = Synthetic Polynucleotide
  source 1..1605: mol_type = other RNA
atggagctgctgatcctcaaggctaacgccatcaccaccatcctgaccgccgtgaccttctgcttcgccagcgggcagaacatcaccgaggagttctaccagagcacttgcagcgccgtgagcaagggctacctgtcagccctgcggaccggctggtacaccagcgtgatcaccatcgagctgagcaacatcaaggagaacaagtgcaacggcaccgacgccaaggtgaagctgatcaagcaggagctggacaagtacaagaacgccgtgaccgagctgcagctgctgatgcagtccacgcccgctaccgggtcaggcagcgccatctgctccggcgtggccgtgtgcaaggtgctgcacctggagggcgaggtgaacaagatcaagagcgccctgctgagcaccaacaaggccgtggtgagcctgagcaacggcgtgagcgtgctgaccttcaaggtgctggacctgaagaactacatcgacaagcagctgctgcccatcctgaacaagcagtcctgctccatctccaacatcgaaactgtgatcgagttccagcagaagaacaaccgcctgctggagatcacccgcgagttcagcgtgaacgccggcgtgaccaccccagtgagcacctacatgctgaccaacagcgagctgctgagcctgatcaacgacatgcccatcaccaacgaccagaagaagctgatgagcaacaacgtgcagatcgtgcgccagcagtcctactctatcatgtgcatcatcaaggaggaggtgctggcctacgtggtgcagctgcccctgtacggcgtgatcgacacaccctgctggaagctgcacacctcccctctgtgcaccactaacaccaaggaggggagcaacatctgcctgacccggaccgaccgcggctggtactgcgacaacgccggcagcgtgagcttcttccctcaggccgaaacctgcaaggtgcagagcaacagggtgttctgcgacaccatgaactcccggaccctgcccagcgaggtgaacctgtgcaacgtggacatcttcaaccccaagtacgattgcaagatcatgacctccaagaccgacgtgagcagctccgtgatcacctccctgggcgccatcgtgagttgctacggcaagaccaagtgcaccgccagcaacaagaaccgcggcatcatcaagaccttcagcaacggctgcgactacgtgtccaacaagggcgtggacaccgtgagcgtgggcaacaccctgtactgcgtgaacaagcaggagggcaagagcctgtacgtgaagggcgagcccatcatcaacttctacgatccgctggtgttcccctccgacgagttcgacgcctccatcagccaggtgaacgagaagatcaaccagagcctggccttcatccgcaagtccgacgagctgctgcacaacgtcaacgccggtaagagcaccaccaacatcatgatcaccacaatcatcatcgtgatcattgtgatcttgctctccttaatcgccgtgggcctgctgctgtactgcaaggcccggagcaccccagtgaccctgagcaaagatcagctgtccggcatcaacaacatcgccttctccaac

SEQ ID NO: 55 - RNA, 1781 nt, synthetic construct
  misc_feature 1..1781: note = Synthetic Polynucleotide
  source 1..1781: mol_type = other RNA
gggaaataagagagaaaagaagagtaagaagaaatataagaccccggcgccgccaccatggagctgctgattctgaaggccaacgccattaccaccattctgaccgccgtgaccttctgcttcgccagcggccagaatattaccgaggagttctaccagtcaacctgcagcgccgtgagcaagggctacctgagcgccctacggaccgggtggtacacctccgtgatcaccattgagctgagcaatattaaggagaataagtgcaacgggaccgacgccaaggtgaagctcatcaagcaggagctggataagtacaagaacgccgtgaccgagctgcagctgctgatgcagagcacaccggccacgggctccggcagcgccatttgctcaggcgtggccgtgtgcaaggtgctgcacctcgagggcgaggtcaataagattaagagcgccctgttgagcaccaataaggccgtggtgagcctgagcaacggcgtgagcgtgctgaccttcaaggtgctggatctcaagaattacattgataagcagctgctgccaattctcaataagcagagctgcagcattagcaatatcgaaaccgtgattgagttccagcagaagaataatcgcctgctggagattacccgcgagttcagcgtcaacgccggggtgaccaccccagtcagcacctacatgctgaccaatagcgagctgctgagcctgattaacgatatgcccattaccaacgaccagaagaagctgatgagcaataacgtgcagattgtgcgccagcagagctacagcattatgtgcattattaaggaggaggtgctggcctacgtggtgcagctgcccctctacggcgtgattgatacgccctgctggaagctgcacaccagcccactgtgcaccaccaataccaaggagggcagcaatatttgcctgacccgcaccgatcgcgggtggtactgcgataacgccggcagcgtgagcttcttcccacaggccgaaacatgcaaggtgcagtccaatcgcgtgttctgcgataccatgaacagccgcaccctgcccagcgaggtgaatctctgcaacgtggatattttcaatccaaagtacgattgcaagattatgaccagcaagaccgacgtgagctccagcgtgattacctcactcggcgccattgtgagctgctacggcaagaccaagtgcaccgcctcaaataagaatcgcggcattattaagaccttcagcaacggctgcgattacgtgagcaataagggcgtggataccgtgagcgtgggaaataccctgtactgcgtgaataagcaggagggcaagagcctgtacgtgaagggcgagccaattattaatttctacgatcccctggtgttccccagcgacgagttcgacgccagcattagccaggtgaacgagaagattaatcagagcctggccttcattcgcaagtccgacgagctcctgcacaacgtgaacgccggcaagagcaccaccaacatcatgattaccaccatcatcattgtgattatcgtgatcctcctcagcctcattgccgtgggcctcctcctgtactgcaaggcccgcagcactcctgtgaccctcagcaaggatcagctgagcggcatcaataatatcgccttcagcaattgataataggctggagcctcggtggcctagcttcttgccccttgggcctccccccagcccctcctccccttcctgcacccgtacccccgtggtctttgaataaagtctgagtgggcggc

SEQ ID NO: 56 - RNA, 1605 nt, synthetic construct
  misc_feature 1..1605: note = Synthetic Polynucleotide
  source 1..1605: mol_type = other RNA
atggagctgctgattctgaaggccaacgccattaccaccattctgaccgccgtgaccttctgcttcgccagcggccagaatattaccgaggagttctaccagtcaacctgcagcgccgtgagcaagggctacctgagcgccctacggaccgggtggtacacctccgtgatcaccattgagctgagcaatattaaggagaataagtgcaacgggaccgacgccaaggtgaagctcatcaagcaggagctggataagtacaagaacgccgtgaccgagctgcagctgctgatgcagagcacaccggccacgggctccggcagcgccatttgctcaggcgtggccgtgtgcaaggtgctgcacctcgagggcgaggtcaataagattaagagcgccctgttgagcaccaataaggccgtggtgagcctgagcaacggcgtgagcgtgctgaccttcaaggtgctggatctcaagaattacattgataagcagctgctgccaattctcaataagcagagctgcagcattagcaatatcgaaaccgtgattgagttccagcagaagaataatcgcctgctggagattacccgcgagttcagcgtcaacgccggggtgaccaccccagtcagcacctacatgctgaccaatagcgagctgctgagcctgattaacgatatgcccattaccaacgaccagaagaagctgatgagcaataacgtgcagattgtgcgccagcagagctacagcattatgtgcattattaaggaggaggtgctggcctacgtggtgcagctgcccctctacggcgtgattgatacgccctgctggaagctgcacaccagcccactgtgcaccaccaataccaaggagggcagcaatatttgcctgacccgcaccgatcgcgggtggtactgcgataacgccggcagcgtgagcttcttcccacaggccgaaacatgcaaggtgcagtccaatcgcgtgttctgcgataccatgaacagccgcaccctgcccagcgaggtgaatctctgcaacgtggatattttcaatccaaagtacgattgcaagattatgaccagcaagaccgacgtgagctccagcgtgattacctcactcggcgccattgtgagctgctacggcaagaccaagtgcaccgcctcaaataagaatcgcggcattattaagaccttcagcaacggctgcgattacgtgagcaataagggcgtggataccgtgagcgtgggaaataccctgtactgcgtgaataagcaggagggcaagagcctgtacgtgaagggcgagccaattattaatttctacgatcccctggtgttccccagcgacgagttcgacgccagcattagccaggtgaacgagaagattaatcagagcctggccttcattcgcaagtccgacgagctcctgcacaacgtgaacgccggcaagagcaccaccaacatcatgattaccaccatcatcattgtgattatcgtgatcctcctcagcctcattgccgtgggcctcctcctgtactgcaaggcccgcagcactcctgtgaccctcagcaaggatcagctgagcggcatcaataatatcgccttcagcaat

SEQ ID NO: 57 - RNA, 1781 nt, synthetic construct
  misc_feature 1..1781: note = Synthetic Polynucleotide
  source 1..1781: mol_type = other RNA
gggaaataagagagaaaagaagagtaagaagaaatataagaccccggcgccgccaccatggagctgctgatcctgaaggccaacgccatcaccaccatcctgaccgccgtgaccttctgcttcgccagcggccagaacattaccgaggagttctaccagtccacctgcagcgccgtgagcaagggctacctgagcgccctgcgcaccgggtggtacaccagcgtgatcaccatcgagctgtccaacatcaaggagaacaagtgcaacggcacagacgccaaggtgaagctgatcaagcaggagctggacaagtacaagaacgccgtgaccgagctccagctgctgatgcagtccaccccggccaccggcagtggaagtgctatctgcagcggggtggccgtgtgcaaggtgctgcacctggagggcgaggtgaacaagatcaagagcgccctgctgagcaccaacaaggccgtggtgagcctgagcaacggggtgtccgtgctgacattcaaggtgctggacctgaagaactacatcgacaagcagctgctgcccatcctcaacaagcagagctgctccatctccaatatcgaaaccgtgatcgagtttcagcagaagaacaaccggctgctggagatcacccgggagttctccgtgaacgccggggtgaccaccccagtgagcacctacatgctgaccaactccgagctgctgagcctgatcaacgatatgccaatcacaaacgatcagaagaagctgatgagcaacaacgtgcagatcgtgcggcagcagtcctacagcatcatgtgcatcatcaaggaggaggtgctggcctacgtggtgcagctgcccctgtacggcgtgatcgacactccttgctggaagctgcacacctcaccgctgtgcaccacgaacactaaggagggcagcaacatctgcctgacccggaccgaccgaggctggtactgcgacaacgccggcagcgtctctttctttccgcaggccgagacgtgcaaggtgcagtccaaccgggtgttctgcgacaccatgaactcccggaccctgccctccgaggtgaacctgtgcaacgtggacattttcaaccccaagtacgactgcaagatcatgaccagtaagaccgacgtgtccagctccgtaatcaccagcctgggcgccatcgtgtcctgttacggcaagaccaagtgtaccgcctccaacaagaaccgaggtatcatcaagaccttctccaacgggtgcgactacgtgtccaacaagggcgtggacaccgtgagcgtgggaaataccctctactgcgtgaacaagcaggaggggaagtccctgtacgtgaagggtgagcccatcatcaacttttacgatcccctggtgttccccagcgacgagttcgacgcatccattagccaggtgaacgagaagatcaaccagagtctggccttcatccgcaagtccgacgagctgctgcacaacgtgaacgctggcaagtcaacaaccaacatcatgatcacgaccatcattatcgtgatcatcgtgatcctgctgtccctgatcgcagtgggcctgctcctgtactgcaaggcccgcagcacacccgtgaccctcagcaaggatcagctcagcggcatcaacaacatcgccttcagcaactgataataggctggagcctcggtggcctagcttcttgccccttgggcctccccccagcccctcctccccttcctgcacccgtacccccgtggtctttgaataaagtctgagtgggcggc

SEQ ID NO: 58 - RNA, 1605 nt, synthetic construct
  misc_feature 1..1605: note = Synthetic Polynucleotide
  source 1..1605: mol_type = other RNA
atggagctgctgatcctgaaggccaacgccatcaccaccatcctgaccgccgtgaccttctgcttcgccagcggccagaacattaccgaggagttctaccagtccacctgcagcgccgtgagcaagggctacctgagcgccctgcgcaccgggtggtacaccagcgtgatcaccatcgagctgtccaacatcaaggagaacaagtgcaacggcacagacgccaaggtgaagctgatcaagcaggagctggacaagtacaagaacgccgtgaccgagctccagctgctgatgcagtccaccccggccaccggcagtggaagtgctatctgcagcggggtggccgtgtgcaaggtgctgcacctggagggcgaggtgaacaagatcaagagcgccctgctgagcaccaacaaggccgtggtgagcctgagcaacggggtgtccgtgctgacattcaaggtgctggacctgaagaactacatcgacaagcagctgctgcccatcctcaacaagcagagctgctccatctccaatatcgaaaccgtgatcgagtttcagcagaagaacaaccggctgctggagatcacccgggagttctccgtgaacgccggggtgaccaccccagtgagcacctacatgctgaccaactccgagctgctgagcctgatcaacgatatgccaatcacaaacgatcagaagaagctgatgagcaacaacgtgcagatcgtgcggcagcagtcctacagcatcatgtgcatcatcaaggaggaggtgctggcctacgtggtgcagctgcccctgtacggcgtgatcgacactccttgctggaagctgcacacctcaccgctgtgcaccacgaacactaaggagggcagcaacatctgcctgacccggaccgaccgaggctggtactgcgacaacgccggcagcgtctctttctttccgcaggccgagacgtgcaaggtgcagtccaaccgggtgttctgcgacaccatgaactcccggaccctgccctccgaggtgaacctgtgcaacgtggacattttcaaccccaagtacgactgcaagatcatgaccagtaagaccgacgtgtccagctccgtaatcaccagcctgggcgccatcgtgtcctgttacggcaagaccaagtgtaccgcctccaacaagaaccgaggtatcatcaagaccttctccaacgggtgcgactacgtgtccaacaagggcgtggacaccgtgagcgtgggaaataccctctactgcgtgaacaagcaggaggggaagtccctgtacgtgaagggtgagcccatcatcaacttttacgatcccctggtgttccccagcgacgagttcgacgcatccattagccaggtgaacgagaagatcaaccagagtctggccttcatccgcaagtccgacgagctgctgcacaacgtgaacgctggcaagtcaacaaccaacatcatgatcacgaccatcattatcgtgatcatcgtgatcctgctgtccctgatcgcagtgggcctgctcctgtactgcaaggcccgcagcacacccgtgaccctcagcaaggatcagctcagcggcatcaacaacatcgccttcagcaac

SEQ ID NO: 59 - RNA, 1597 nt, synthetic construct
  misc_feature 1..1597: note = Synthetic Polynucleotide
  source 1..1597: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgggaggcggtagcggtggcggatccggcggtggaagcggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccactttccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 60 - RNA, 1431 nt, synthetic construct
  misc_feature 1..1431: note = Synthetic Polynucleotide
  source 1..1431: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgggaggcggtagcggtggcggatccggcggtggaagcggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccactttccgccatcggtggctatatcccagaagccccaagagacggacaagcgtacgtccggaaagatggtgagtgggtcctcctctctacctttctt

SEQ ID NO: 61 - protein, 477 aa, synthetic construct
  REGION 1..477: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMGGGSGGGSGGGSGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLSAIGGYIPEAPRDGQAYVRKDGEWVLLSTFL

SEQ ID NO: 62 - RNA, 1702 nt, synthetic construct
  misc_feature 1..1702: note = Synthetic Polynucleotide
  source 1..1702: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacagagctaggagggagttgcctaggtttatgaactacactctcaacaacgcgaagaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccactttccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 63 - RNA, 1536 nt, synthetic construct
  misc_feature 1..1536: note = Synthetic Polynucleotide
  source 1..1536: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacagagctaggagggagttgcctaggtttatgaactacactctcaacaacgcgaagaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccactttccgccatcggtggctatatcccagaagccccaagagacggacaagcgtacgtccggaaagatggtgagtgggtcctcctctctacctttctt

SEQ ID NO: 64 - protein, 512 aa, synthetic construct
  REGION 1..512: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLSAIGGYIPEAPRDGQAYVRKDGEWVLLSTFL

SEQ ID NO: 65 - RNA, 1798 nt, synthetic construct
  misc_feature 1..1798: note = Synthetic Polynucleotide
  source 1..1798: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacagagctaggagggagttgcctaggtttatgaactacactctcaacaacgcgaagaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagttgcgaattctgcgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 66 - RNA, 1632 nt, synthetic construct
  misc_feature 1..1632: note = Synthetic Polynucleotide
  source 1..1632: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacagagctaggagggagttgcctaggtttatgaactacactctcaacaacgcgaagaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagttgcgaattctgcgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 67 - protein, 544 aa, synthetic construct
  REGION 1..544: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSCEFCASISQVNEKINQSLAFIRKSDELLSAIGGYIPEAPRDGQAYVRKDGEWVLLSTFL

SEQ ID NO: 68 - RNA, 1693 nt, synthetic construct
  misc_feature 1..1693: note = Synthetic Polynucleotide
  source 1..1693: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgggaggcggtagcggtggcggatccggcggtggaagcggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagttgcgaattctgcgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 69 - RNA, 1527 nt, synthetic construct
  misc_feature 1..1527: note = Synthetic Polynucleotide
  source 1..1527: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgggaggcggtagcggtggcggatccggcggtggaagcggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagttgcgaattctgcgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 70 - protein, 509 aa, synthetic construct
  REGION 1..509: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMGGGSGGGSGGGSGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSCEFCASISQVNEKINQSLAFIRKSDELLSAIGGYIPEAPRDGQAYVRKDGEWVLLSTFL

SEQ ID NO: 71 - RNA, 1888 nt, synthetic construct
  misc_feature 1..1888: note = Synthetic Polynucleotide
  source 1..1888: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacagagctaggagggagttgcctaggtttatgaactacactctcaacaacgcgaagaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagttgcgaattctgcgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 72 - RNA, 1722 nt, synthetic construct
  misc_feature 1..1722: note = Synthetic Polynucleotide
  source 1..1722: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacagagctaggagggagttgcctaggtttatgaactacactctcaacaacgcgaagaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagttgcgaattctgcgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 73 - protein, 574 aa, synthetic construct
  REGION 1..574: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSCEFCASISQVNEKINQSLAFIRKSDELLHNVNAGKSTTNIMITTIIIVIIVILLSLIAVGLLLYCKARSTPVTLSKDQLSGINNIAFSN

SEQ ID NO: 74 - RNA, 1783 nt, synthetic construct
  misc_feature 1..1783: note = Synthetic Polynucleotide
  source 1..1783: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgggaggcggtagcggtggcggatccggcggtggaagcggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagttgcgaattctgcgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 75 - RNA, 1617 nt, synthetic construct
  misc_feature 1..1617: note = Synthetic Polynucleotide
  source 1..1617: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgggaggcggtagcggtggcggatccggcggtggaagcggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagttgcgaattctgcgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 76 - protein, 539 aa, synthetic construct
  REGION 1..539: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMGGGSGGGSGGGSGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSCEFCASISQVNEKINQSLAFIRKSDELLHNVNAGKSTTNIMITTIIIVIIVILLSLIAVGLLLYCKARSTPVTLSKDQLSGINNIAFSN

SEQ ID NO: 77 - RNA, 57 nt, synthetic construct
  misc_feature 1..57: note = Synthetic Polynucleotide
  source 1..57: mol_type = other RNA
gggaaataagagagaaaagaagagtaagaagaaatataagaccccggcgccgccacc

SEQ ID NO: 78 - RNA, 119 nt, synthetic construct
  misc_feature 1..119: note = Synthetic Polynucleotide
  source 1..119: mol_type = other RNA
tgataataggctggagcctcggtggcctagcttcttgccccttgggcctccccccagcccctcctccccttcctgcacccgtacccccgtggtctttgaataaagtctgagtgggcggc

SEQ ID NO: 79 - protein, 30 aa, synthetic construct
  REGION 1..30: note = Synthetic Polypeptide
MDSKGSSQKGSRLLLLLVVSNLLLPQGVVG

SEQ ID NO: 80 - protein, 18 aa, synthetic construct
  REGION 1..18: note = Synthetic Polypeptide
MDWTWILFLVAAATRVHS

SEQ ID NO: 81 - protein, 20 aa, synthetic construct
  REGION 1..20: note = Synthetic Polypeptide
METPAQLLFLLLLWLPDTTG

SEQ ID NO: 82 - protein, 24 aa, synthetic construct
  REGION 1..24: note = Synthetic Polypeptide
MLGSNSGQRVVFTILLLLVAPAYS

SEQ ID NO: 83 - protein, 17 aa, synthetic construct
  REGION 1..17: note = Synthetic Polypeptide
MKCLLYLAFLFIGVNCA

SEQ ID NO: 84 - protein, 15 aa, synthetic construct
  REGION 1..15: note = Synthetic Polypeptide
MWLVSLAIVTACAGA

SEQ ID NO: 85 - skipped

SEQ ID NO: 86 - RNA, 11 nt, synthetic construct
  misc_feature 1..11: note = Synthetic Polynucleotide
  source 1..11: mol_type = other RNA
gggatcctacc

SEQ ID NO: 87 - skipped

SEQ ID NO: 88 - RNA, 1722 nt, synthetic construct
  misc_feature 1..1722: note = Synthetic Polynucleotide
  source 1..1722: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccaataacagagctaggagggagttgcctaggtttatgaactacactctcaacaacgcgaaaaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 89 - protein, 574 aa, synthetic construct
  REGION 1..574: note = Synthetic Polypeptide
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 90 - RNA, 1888 nt, synthetic construct
  misc_feature 1..1888: note = Synthetic Polynucleotide
  source 1..1888: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataaaaacgctgtgaccgagctgcagttattgatgcagagtacacctgccaccaataacagagctaggagggagttgcctaggtttatgaactacactctcaacaacgcgaaaaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaaaaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 91 - RNA, 1722 nt, synthetic construct
  misc_feature 1..1722: note = Synthetic Polynucleotide
  source 1..1722: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacagagctaggagggagttgcctaggtttatgaactacactctcaacaacgcgaagaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaat

SEQ ID NO: 92 - RNA, 1888 nt, synthetic construct
  misc_feature 1..1888: note = Synthetic Polynucleotide
  source 1..1888: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgagtgcgctccgcacaggatggtacacctccgtgatcaccatcgagctcagcaatattaaagagaacaagtgcaatggtaccgacgctaaagtcaaacttatcaagcaggaactcgacaaatataagaacgctgtgaccgagctgcagttattgatgcagagtacacctgccaccaataacagagctaggagggagttgcctaggtttatgaactacactctcaacaacgcgaagaaaaccaatgtgacgctatccaagaaacggaagaggaggttcctggggtttcttttaggggtgggctctgccattgcttccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaataagtaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctctgactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactacgttaacaaacaggaaggcaaatccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgcataacgtgaatgctgggaaatctacaaccaacatcatgatcactaccatcattattgtgattatcgtaattctgctatccttgattgctgtcgggctgcttctgtactgtaaggccagatcgacgcctgtgaccctttcaaaagaccaacttagcggtatcaataatattgcctttagcaattgataataggctggagcctcggtggccatgcttcttgccccttgggcctccccccagcccctcctccccttcctgcacccgtacccccgtggtctttgaataaagtctgagtgggcggc

SEQ ID NO: 93 - DNA, 25 bp, synthetic construct
  misc_feature 1..25: note = Synthetic Polynucleotide
  source 1..25: mol_type = other DNA
ctcaatttcctcacttctccagtgt

SEQ ID NO: 94 - DNA, 24 bp, synthetic construct
  misc_feature 1..24: note = Synthetic Polynucleotide
  source 1..24: mol_type = other DNA
cttgattcctcggtgtacctctgt

SEQ ID NO: 95 - DNA, 25 bp, synthetic construct
  misc_feature 1..25: note = Synthetic Polynucleotide
  source 1..25: mol_type = other DNA
tcccattatgcctaggccagcagca

SEQ ID NO: 96 - RNA, 1515 nt, synthetic construct
  misc_feature 1..1515: note = Synthetic Polynucleotide
  source 1..1515: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagatcaagtgcaatggtaccgacgctaaagtcaaacttatcaagcaggaactcgacaaatataagaacgctgtgaccgacctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 97 - RNA, 1518 nt, synthetic construct
  misc_feature 1..1518: note = Synthetic Polynucleotide
  source 1..1518: mol_type = other RNA
atggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagatcaagtgcaatggtaccgacgctaaagtcaaacttatcaagcaggaactcgacaaatataagaacgctgtgaccgacctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctt

SEQ ID NO: 98 - RNA, 1681 nt, synthetic construct
  misc_feature 1..1681: note = Synthetic Polynucleotide
  source 1..1681: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagatcaagtgcaatggtaccgacgctaaagtcaaacttatcaagcaggaactcgacaaatataagaacgctgtgaccgacctgcagttattgatgcagagtacacctgccaccgggtcgggctctgccatttgctccggcgtggctgtatgtaaagttctccacctcgagggagaggttaataagattaagtcggccctgctgagtactaacaaagcagtggtgtcgctgagtaacggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagaacagaggcataattaaaaccttttcaaatggctgtgactatgtgtcgaataagggcgtcgacacggtctcagtagggaataccctctactgcgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 99 - RNA, 1684 nt, synthetic construct
  misc_feature 1..1684: note = Synthetic Polynucleotide
  source 1..1684: mol_type = other RNA
gggaaataagagagaaaagaagagtaagaagaaatataagagccaccatggaactgctcattttgaaggcaaacgctatcacgacaatactcactgcagtgaccttctgttttgcctcaggccagaacataaccgaggagttttatcaatctacatgcagcgctgtatctaaaggctacctgggcgcgctccgcacaggatggtacacctccgtgatcaccatcgagctcagcaatattaaagagatcaagtgcaatggtaccgacgctaaagtcaaacttatcaagcaggaactcgacaaatataagaacgctgtgaccgacctgcagttattgatgcagagtacacctgccaccgggtcgggctctgccattgcttccggcgtggctgtatgtaaagttctccacctcgagggagaggttaataagattaagtcggccctgctgagtactaacaaagcagtggtgtcgctgagtggctgcggagtaagtgtgttaacatttaaggtgctggacctcaagaattatattgacaaacagttgcttcctattctaaacaaacagagctgttcaatacccaatattgaaactgttattgagtttcagcagaagaacaacaggcttcttgagattacacgcgagttcagtgtcaatgccggcgttacaacacccgtgtctacctacatgctgacgaattctgagcttctctctctcataaacgacatgcccattacgaatgaccaaaagaaacttatgtccaacaacgtgcagattgtgcgacagcaatcctatagcattatgtgtatcatcaaggaagaggtactcgcttatgttgtgcagctaccactctatggtgtgattgacaccccctgttggaagctgcataccagtccactctgcaccactaacacaaaggaagggagcaatatttgcctcactcgaaccgacagggggtggtattgcgataatgcgggctccgtgtccttctttccacaggctgaaacttgtaaggtacagtcaaaccgcgtgttctgtgatactatgaattctcgtactcttcccagcgaggttaatctctgcaacgtcgacattttcaatcctaaatatgactgcaagatcatgaccagcaagaccgacgtctccagctcagtaatcactagcctaggggccattgtaagctgctatggcaaaaccaagtgtactgcctctaataagtgcagaggcataattaaaaccttttcaaatggctgtgactatgtgtcgaataagggcgtcgacacggtctcagtagggaataccctctactacgttaacaaacaggaaggccagtccctttatgtaaagggcgagcccatcataaatttctacgacccacttgtgttccccagtgatgaattcgatgcatcaatctcccaggtgaacgaaaagatcaatcaatcccttgcttttatacgaaagtcagatgaactcctgtccgccatcggtggctatatcccagaagccccaagagacggacaagcgtacgtccggaaagatggtgagtgggtcctcctctctacctttctttgataataggctggagcctcggtggccatgcttcttgccccttgggcctccccccagcccctcctccccttcctgcacccgtacccccgtggtctttgaataaagtctgagtgggcggc

SEQ ID NO: 100 - protein, 25 aa, synthetic construct
  REGION 1..25: note = Synthetic Polypeptide
MELLILKANAITTILTAVTFCFASG